(19) United States
(12) Patent Application Publication
(10) Pub. No.: US 2021/0128731 A1
(43) Pub. Date: May 6, 2021

(54) NANOPARTICLES FOR BORON NEUTRON CAPTURE THERAPY AND FOR DIAGNOSING, DETECTING, AND TREATING CANCER

(71) Applicants: CEDARS-SINAI MEDICAL CENTER, Los Angeles, CA (US); THE REGENTS OF THE UNIVERSITY OF CALIFORNIA, Oakland, CA (US)

(72) Inventors: J. Manuel Perez, West Hollywood, CA (US); Alexander M. Spokoyny, Los Angeles, CA (US); James Teh, La Habra, CA (US); Nicholas Bernier, Los Angeles, CA (US); Keith L. BLACK, Los Angeles, CA (US); Leland Chung, Beverly Hills, CA (US); Yi Zhang, Los Angeles, CA (US)

(73) Assignees: CEDARS-SINAI MEDICAL CENTER, Los Angeles, CA (US); THE REGENTS OF THE UNIVERSITY OF CALIFORNIA, Oakland, CA (US)

(21) Appl. No.: 17/047,647

(22) PCT Filed: Apr. 18, 2019
(86) PCT No.: PCT/US2019/028179

§ 371 Date: Oct. 14, 2020

Related U.S. Application Data
(60) Provisional application No. 62/659,449, filed on Apr. 18, 2018.
(60) Provisional application No. 62/731,575, filed on Sep. 14, 2018.

(51) Int. Cl. A61K 41/00; A61K 49/00; A61K 47/54; A61K 47/61; A61K 47/69; A61K 33/22; A61K 47/62; A61K 9/51

(52) U.S. Cl. CPC A61K 41/0095; A61K 49/0093; A61K 49/0054; A61K 47/542; A61K 9/5161; A61K 47/6923; A61K 33/22; A61K 47/62; A61K 9/5115; A61K 47/61

(57) ABSTRACT

The invention provides a nanovehicle, comprising: a core, wherein the core comprises at least one iron oxide: a shell surrounding the core, wherein the shell comprises at least one polymer; and at least one boron cluster. The invention provides a method of treating a disease, disorder, or disease condition in a subject, comprising administering a therapeutically effective amount of the nanovehicle to the subject; and radiating the nanovehicle with neutrons. In various embodiments, the invention provides a method for detecting a cancer in a subject, comprising administering an effective amount of at least one nanoparticle of the present invention to the subject. The invention also provides the nanovehicles (e.g., nanoparticles) described herein in the form of various pharmaceutical formulations. The invention provides a kit, the kit comprises: a quantity of the nanovehicle (e.g., nanoparticle) described herein.

### CROSS-REFERENCE TO RELATED APPLICATIONS

This application claims priority under 35 U.S.C. § 119(e) to U.S. provisional patent application No. 62/659,449 filed Apr. 18, 2018, and U.S. provisional patent application No. 62/731,575 filed Sep. 14, 2018, the entirety of each of which are hereby incorporated by reference.

### STATEMENT REGARDING FEDERALLY SPONSORED RESEARCH OR DEVELOPMENT

This invention was made with government support under Grant Nos. GM124746 and EB019288 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 17, 2019, is named SequenceListing-065472-000740WO00_ST25.txt and is 1,933 bytes in size.

### FIELD OF THE INVENTION

The invention relates to nanoparticles comprising boron clusters and methods using the nanoparticles for diagnosing, detecting, and treating cancer.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Many people suffer from cancer and require treatment. As such there is a need for safer alternatives to many of the single-component radiation and chemotherapy approaches used for the treatment of cancer. There is also a need for improved cancer diagnosis and detection, and for improved therapies for the treatment of cancer. The present invention addresses those needs.

### SUMMARY OF THE INVENTION

The following embodiments and aspects thereof are described and illustrated in conjunction with systems, compositions, articles of manufacture, and methods which are meant to be exemplary and illustrative, not limiting in scope.

In various embodiments, the present invention provides a nanovehicle, comprising: a core, wherein the core comprises at least one iron oxide; a shell surrounding the core, wherein the shell comprises at least one polymer; and at least one boron cluster.

In various embodiments, the present invention provides a method of treating, reducing the severity of and/or slowing the progression of a disease, disorder, or disease condition in a subject, comprising: providing at least one nanovehicle, wherein the at least one nanovehicle comprises: a core, wherein the core comprises at least one iron oxide; a shell surrounding the core, wherein the shell comprises at least one polymer; and at least one boron cluster; administering a therapeutically effective amount of the at least one nanovehicle to the subject; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject. In some embodiments, the nanovehicle further comprises at least one targeting moiety attached to the shell. In some embodiments, the nanovehicle is a nanoparticle.

In various embodiments, the present invention provides a nanoparticle, comprising: a core, wherein the core comprises at least one iron oxide; a shell surrounding the core, wherein the shell comprises at least one polymer; at least one boron cluster; and at least one targeting moiety attached to the shell.

In various embodiments, the present invention provides a method for detecting a cancer in a subject, comprising: administering an effective amount of at least one nanoparticle of the present invention to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject.

In various embodiments, the present invention provides a method for detecting a cancer in a subject, comprising: administering an effective amount of at least one probe of the present invention to the subject, thereby contacting a tissue of the subject with the at least one probe such that the at least one probe binds to the tissue; and detecting the at least one probe bound to the tissue, wherein the presence of the at least one probe bound to the tissue is indicative of the cancer in the subject.

In various embodiments, the present invention provides a method of treating, reducing the severity of and/or slowing the progression of cancer in a subject, comprising: providing at least one nanovehicle, wherein the at least one nanovehicle comprises: a core, wherein the core comprises at least one iron oxide; a shell surrounding the core, wherein the shell comprises at least one polymer; at least one boron cluster; and at least one targeting ligand attached to the shell; administering a therapeutically effective amount of the at least one nanovehicle to the subject, thereby contacting a tissue of the subject with the at least one nanovehicle, wherein the tissue is selected from the group consisting of cancerous tissue, non-cancerous tissue, and combinations thereof, and wherein the nanovehicle selectively binds to the cancerous tissue; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the cancer in the subject.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, a boron neutron capture therapy (BNCT) scheme.

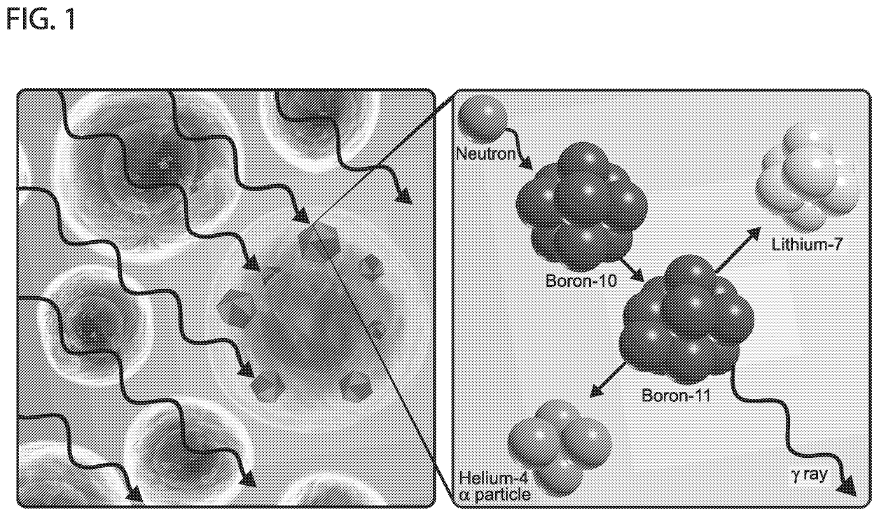

FIG. 2 depicts non-limiting examples of boron containing compounds.

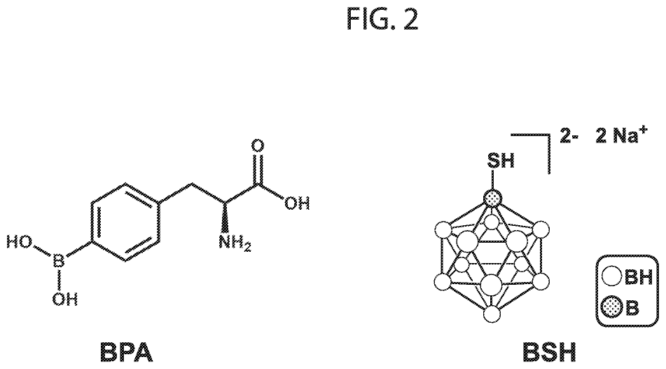

FIG. 3 depicts in accordance with various embodiments of the invention, a schematic diagram of encapsulation of boron clusters with Feraheme nanoparticles. Picture of Feraheme bottle (top left). Schematic diagram of Feraheme structure (top right), showing the carboxymethyl dextran coating that facilitates boron cluster encapsulation. Cartoon depiction of encapsulation of boron clusters into Feraheme (bottom).

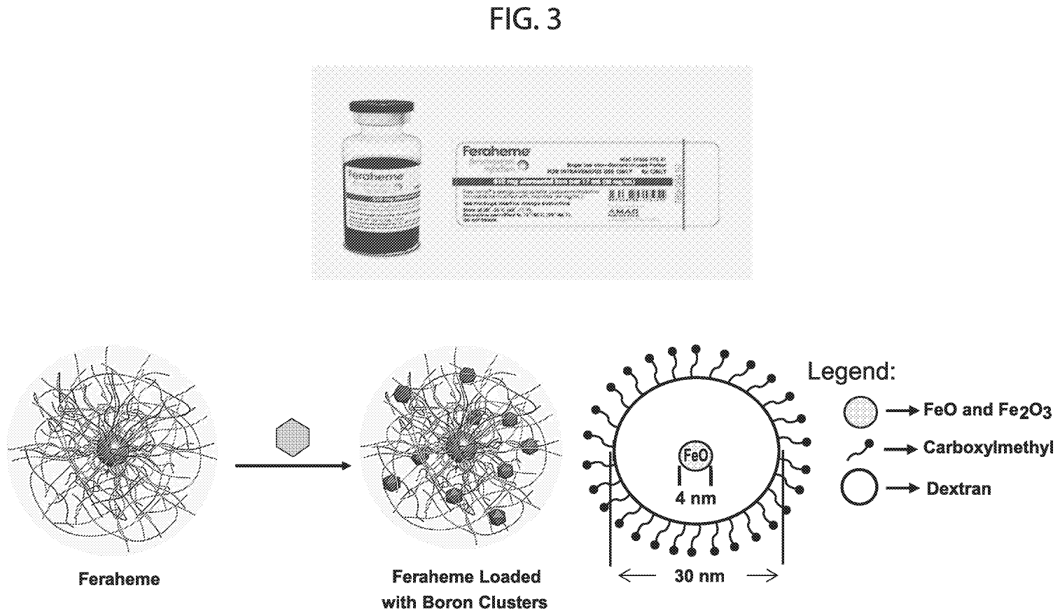

FIG. 4 depicts in accordance with various embodiments of the invention, a non-limiting example of a boron cluster Na₂B₁₂H₁₂ for boron neutron capture therapy (BNCT).

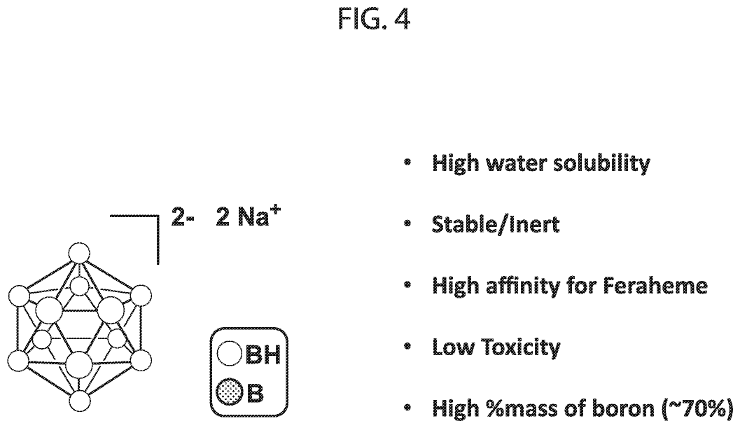

FIG. 5 depicts in accordance with various embodiments of the invention, ICP measurements of Feraheme-loaded boron clusters. Prepared 100 mg Na₂B₁₂H₁₂ (BC2) NP batch (1M cluster loading). Measured both B (boron) and Fe (iron) content by ICP.

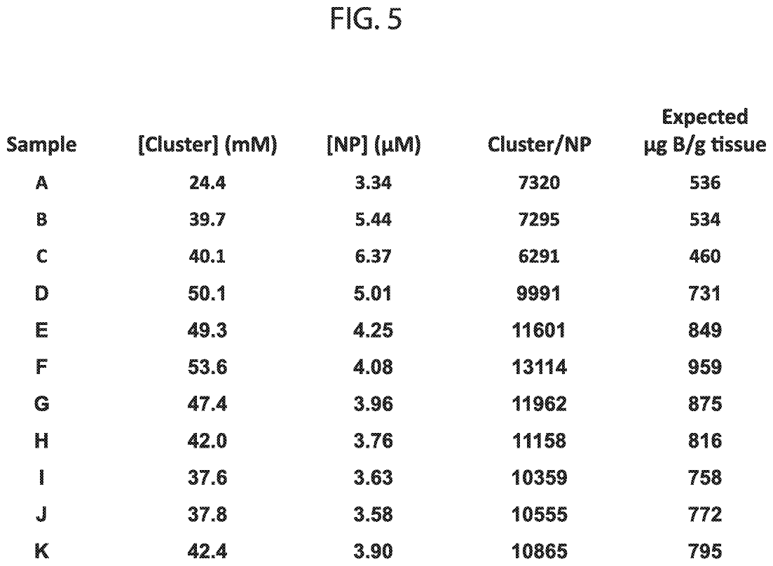

FIG. 6 depicts in accordance with various embodiments of the invention, ICP measurements of Feraheme-loaded boron clusters.

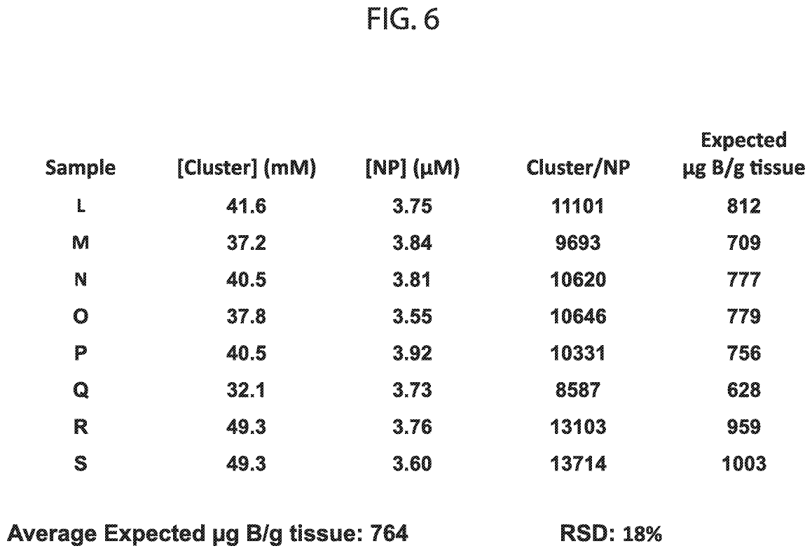

FIG. 7 depicts in accordance with various embodiments of the invention, differences between Feraheme bottles

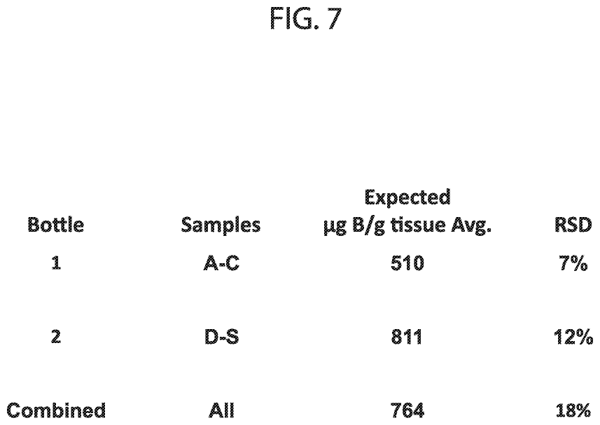

FIG. 8 depicts in accordance with various embodiments of the invention, a graph showing expected boron concentrations in tissue based on ICP measurements.

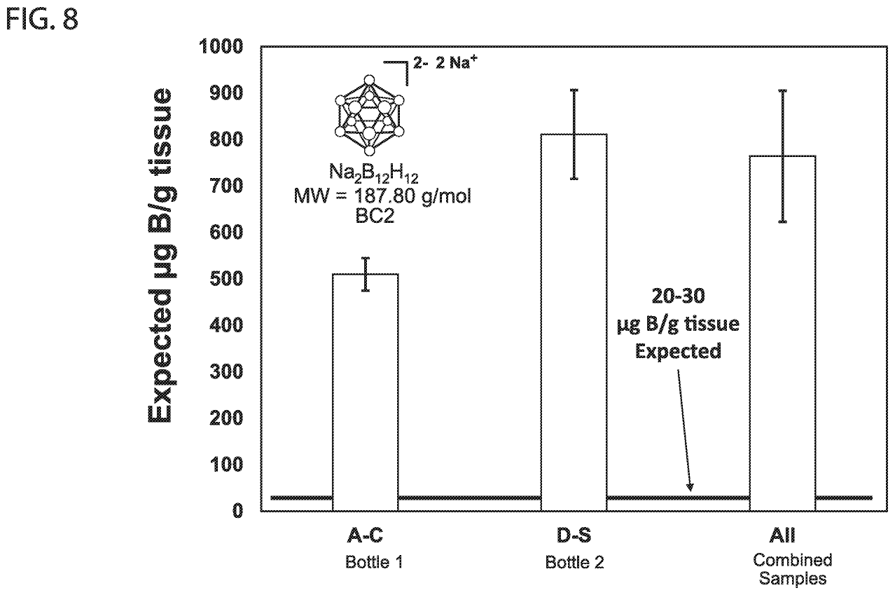

FIG. 9 depicts in accordance with various embodiments of the invention, a summary of NMR leaching studies demonstrating high stability of the Feraheme nanoparticles loaded with boron clusters. Leaching studies were conducted via NMR to verify that the boron clusters are actually contained in the Feraheme NP and that the system is stable in varied conditions.

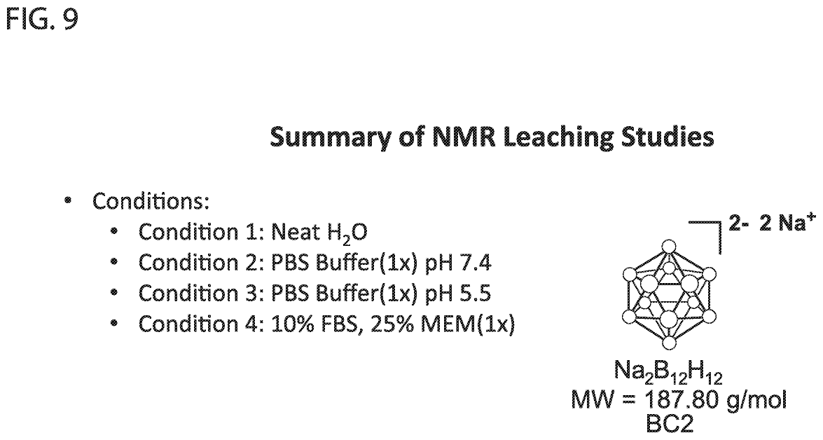

FIG. 10 depicts in accordance with various embodiments of the invention, a ¹¹B N/R spectrum according to Condition 1: Neat H₂O from NMR leaching study. Spectra Referenced Internally to 0.4M B(OH)₃ in D₂O.

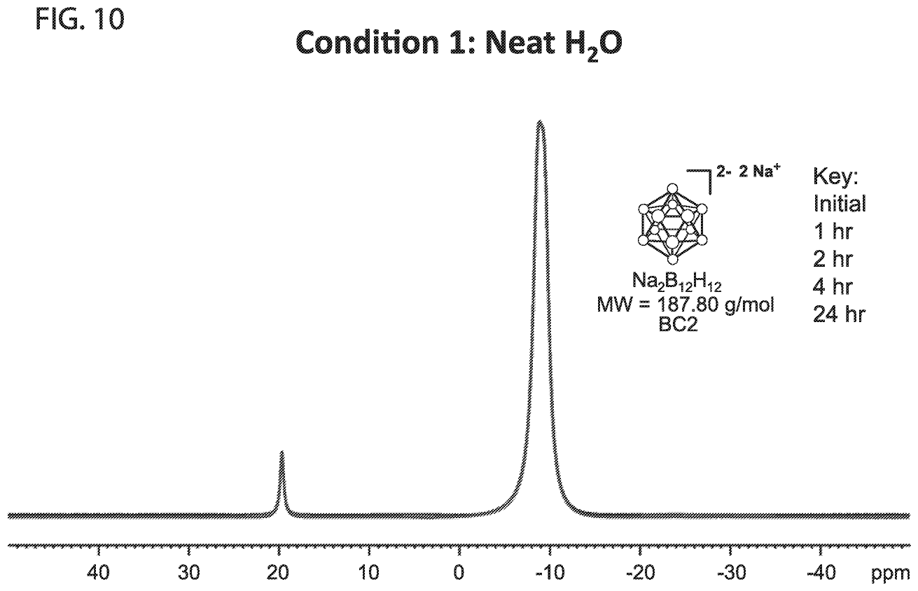

FIG. 11 depicts in accordance with various embodiments of the invention, a ¹¹B N/R spectrum according to Condition 2: PBS(1×) pH 7.4 from N/R leaching study. Spectra Referenced Internally to 0.4M B(OH)₃ in D₂O.

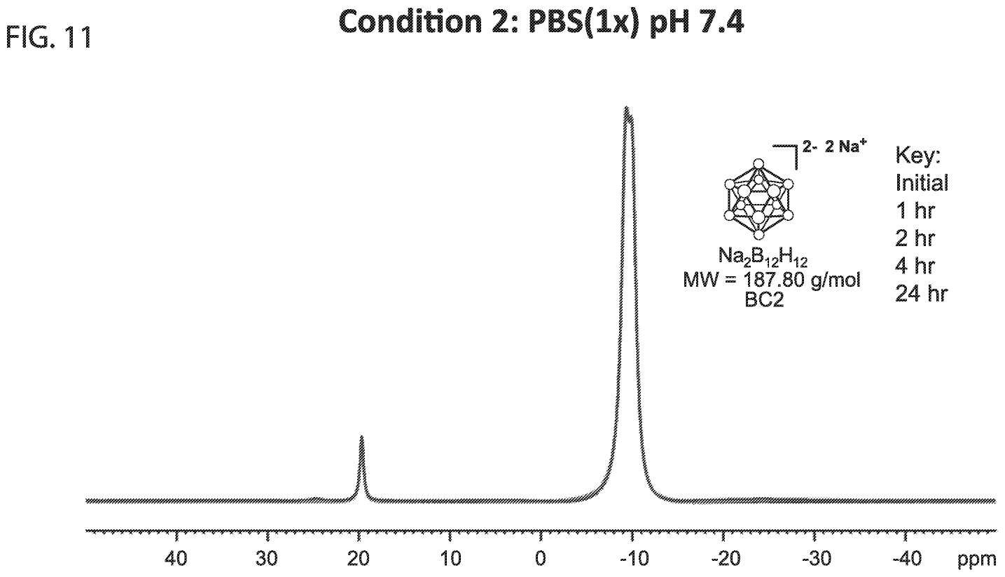

FIG. 12 depicts in accordance with various embodiments of the invention, a ¹¹B N/R spectrum according to Condition 3: PBS(1×) pH 5.5 from N/R leaching study. Spectra Referenced Internally to 0.4M B(OH)₃ in D₂O.

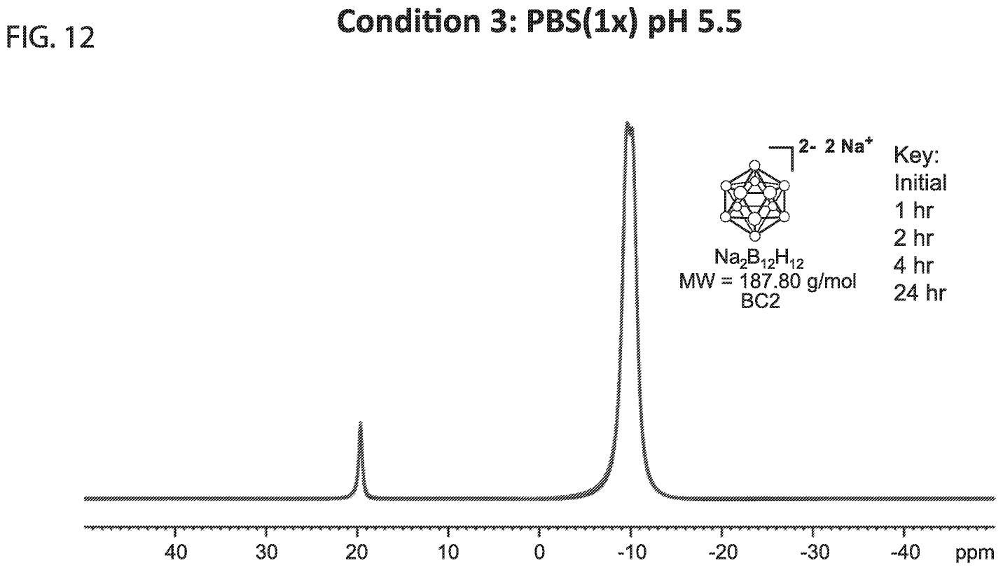

FIG. 13 depicts in accordance with various embodiments of the invention, a ¹¹B N/R spectrum according to Condition 4: 10% FBS, 25% MEM(1×) from NMR leaching study. Spectra Referenced Internally to 0.4M B(OH)₃ in D₂O.

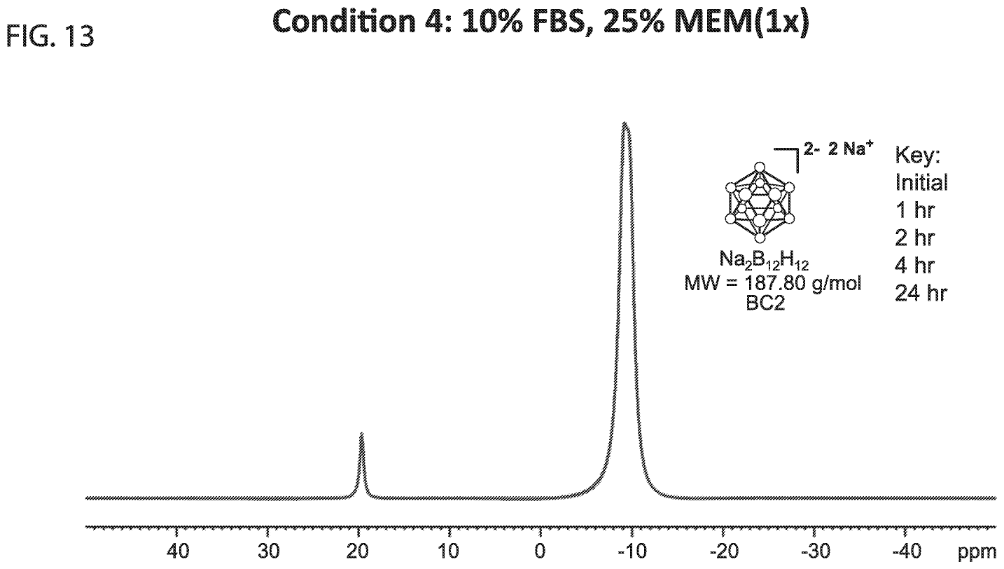

FIG. 14 depicts in accordance with various embodiments of the invention, a ¹¹B N/R spectrum of Feraheme loaded Na₂B₁₂H₁₂ vs. free Na₂B₁₂H₁₂. Spectra Referenced Internally to 0.4M B(OH)₃ in D₂O.

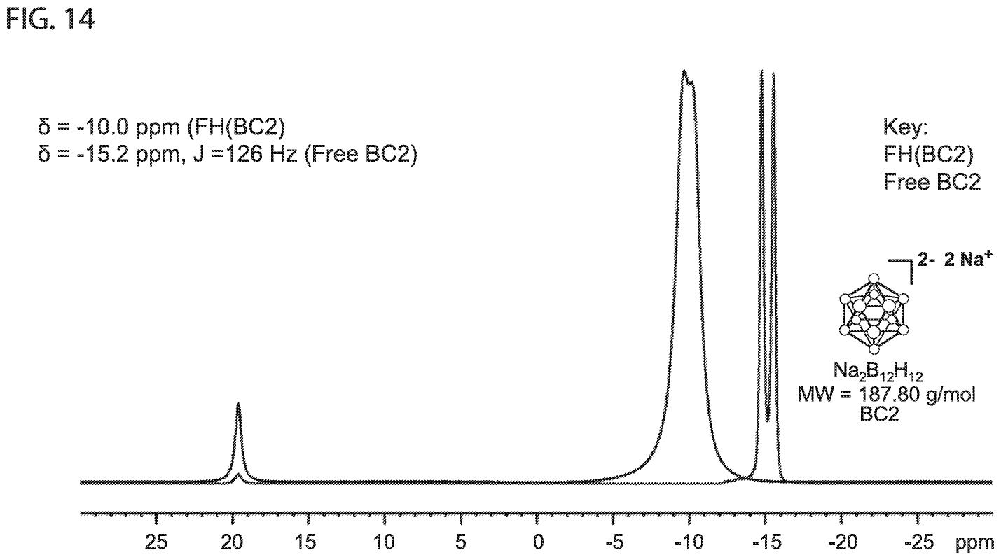

FIG. 15 depicts in accordance with various embodiments of the invention, a ¹¹B NMR titration of Feraheme with Na₂B₁₂H₁₂. Spectra Referenced Internally to 0.4M B(OH)₃ in D₂O.

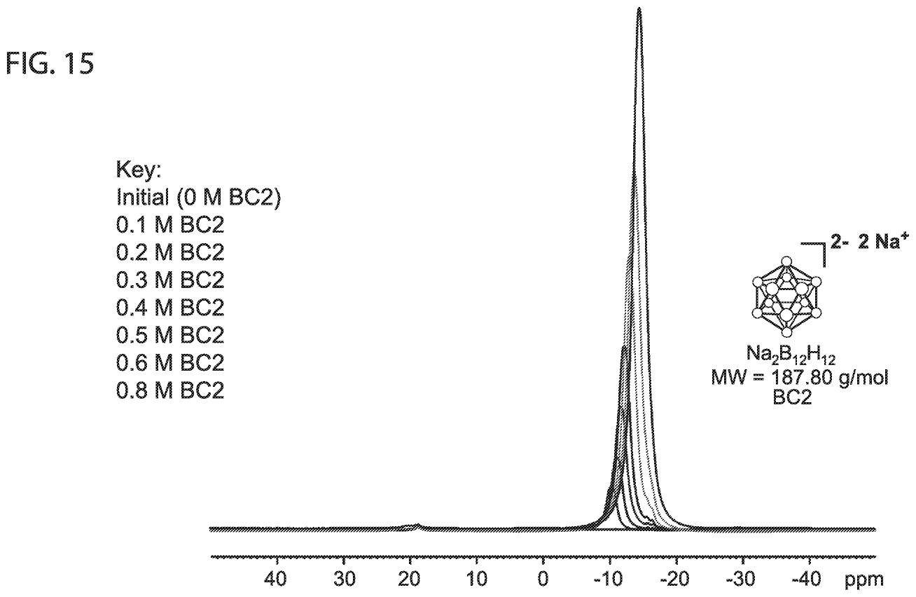

FIG. 16 depicts in accordance with various embodiments of the invention, a ¹¹B NMR titration of Feraheme with Na₂B₁₂H₁₂. Spectra Referenced Internally to 0.4M B(OH)₃ in D₂O.

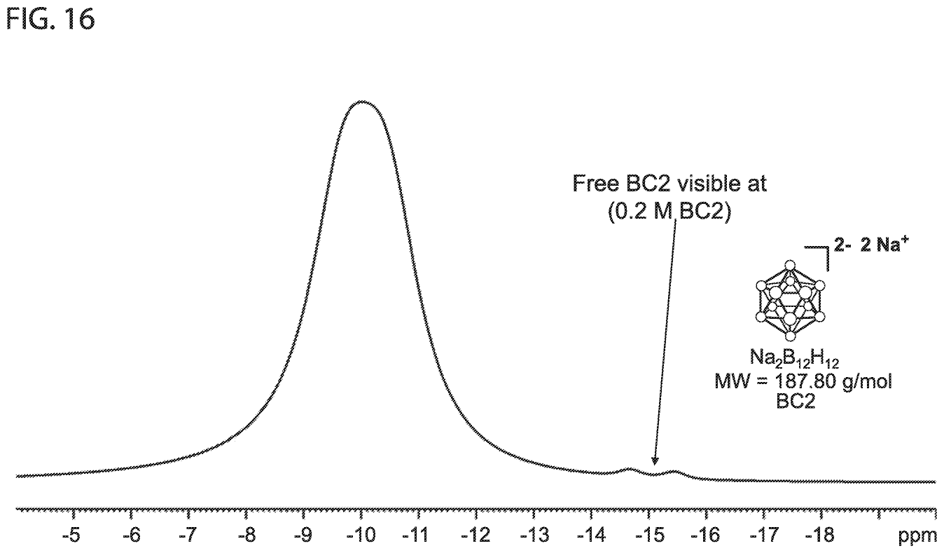

FIG. 17 depicts in accordance with various embodiments of the invention, non-limiting examples of boron clusters.

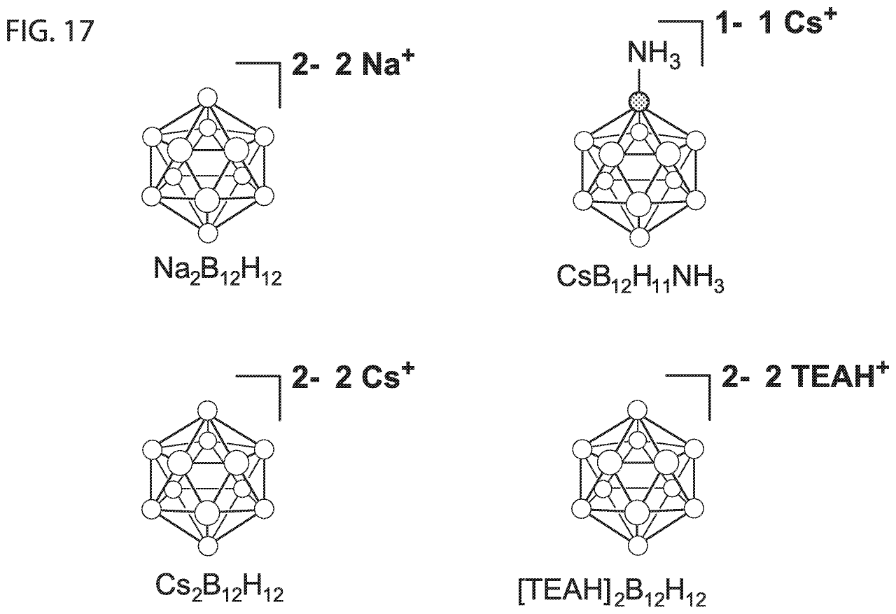

FIG. 18 depicts in accordance with various embodiments of the invention, non-limiting examples of boron clusters. Homoperfunctionalization of closo-[B₁₂H₁₂]²⁻. Cations omitted for clarity. (a) Alkylation. Peralkylation of dodecaborate can be achieved by refluxing [NⁿBu₄]₂[B₁₂H₁₂] with neat AlMe₃ and iodomethane over 11 days to form [NⁿBu₄]₂[B₁₂Me₁₂]. (b) Halogenation. (c) Hydroxylation. (d) Carbamation. (e) Carbonation. (f) Esterification. (g). Etherification.

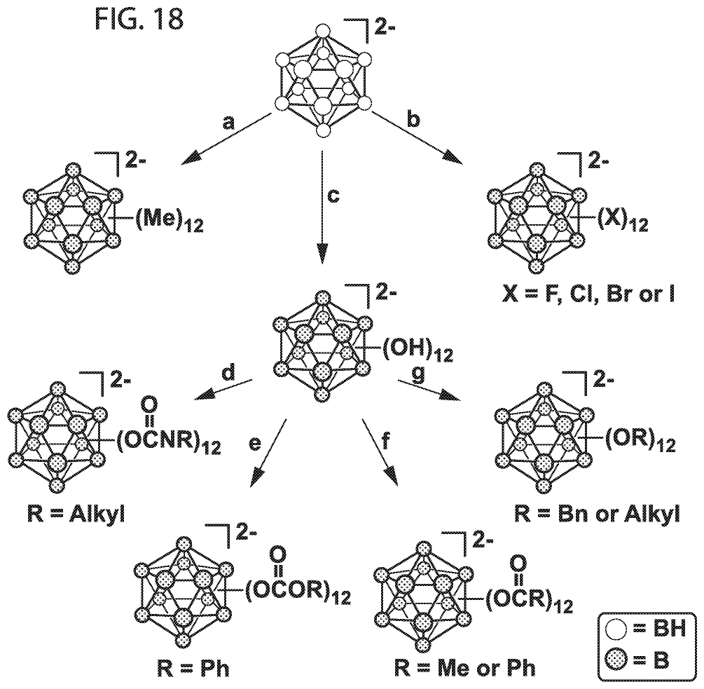

FIG. 19 depicts in accordance with various embodiments of the invention, non-limiting examples of boron clusters. Established routes for the heteroperfunctionalization of closo-[B₁₂H₁₂]²⁻. Cations have been omitted for clarity.

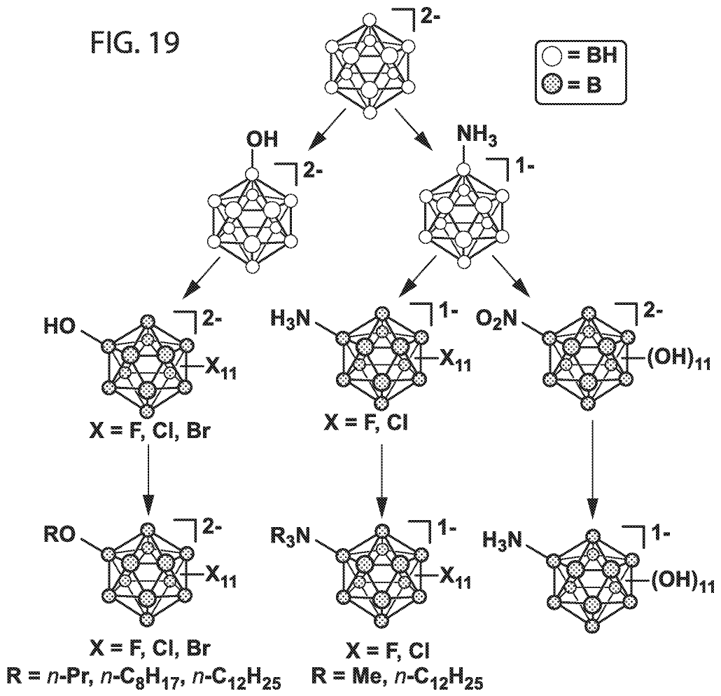

FIG. 20 depicts in accordance with various embodiments of the invention, non-limiting examples of boron clusters. Summary of reactivity of tetrachloro-closo-tetraborate.

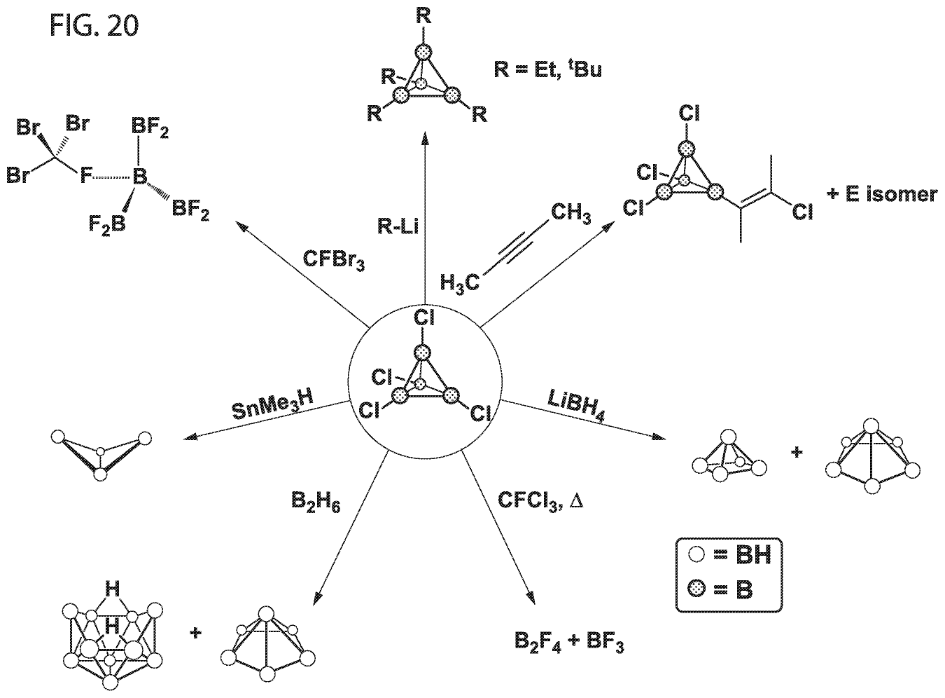

FIG. 21 depicts in accordance with various embodiments of the invention, non-limiting examples of boron clusters. Reactivity of closo-[B₆H6]²⁻.

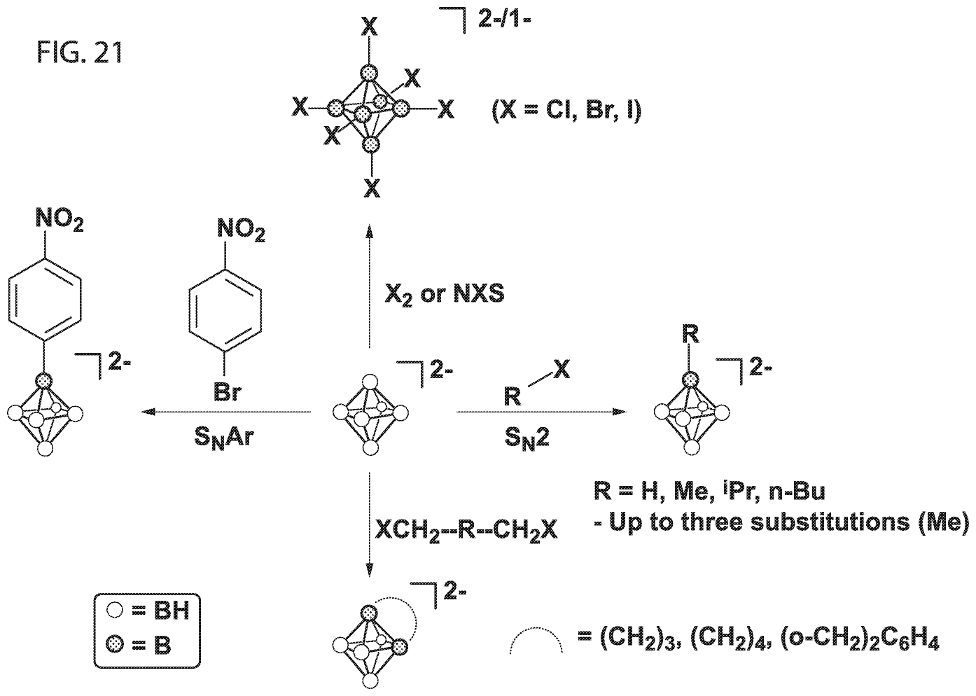

FIG. 22A-FIG. 22B depicts in accordance with various embodiments of the invention, non-limiting examples of boron clusters. FIG. 22A synthesis of perfunctionalized B₆ boron clusters featuring direct B—C bonds. FIG. 22B X-ray crystal structure of closo-[B₆(CH₂-4-BrC₆H₄)₆H^{fac}]—.

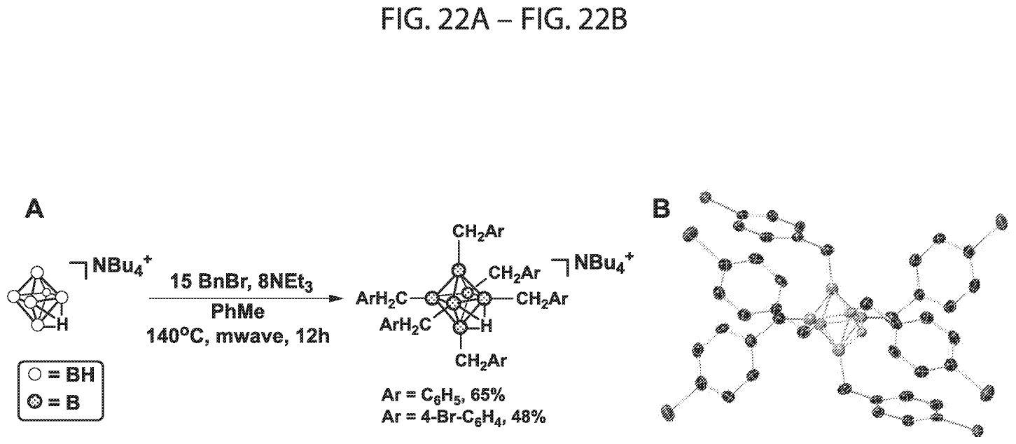

FIG. 23A-FIG. 23B depicts in accordance with various embodiments of the invention, non-limiting examples of boron clusters. FIG. 23A X-ray crystal structure of [(PPh₃)₂N][B₁₂Me₁₂](the [(PPh₃)₂N]⁺ counterion was omitted for clarity. FIG. 23B singly occupied molecular orbital and spin-density localization (PBE/D3BJ:TZP).

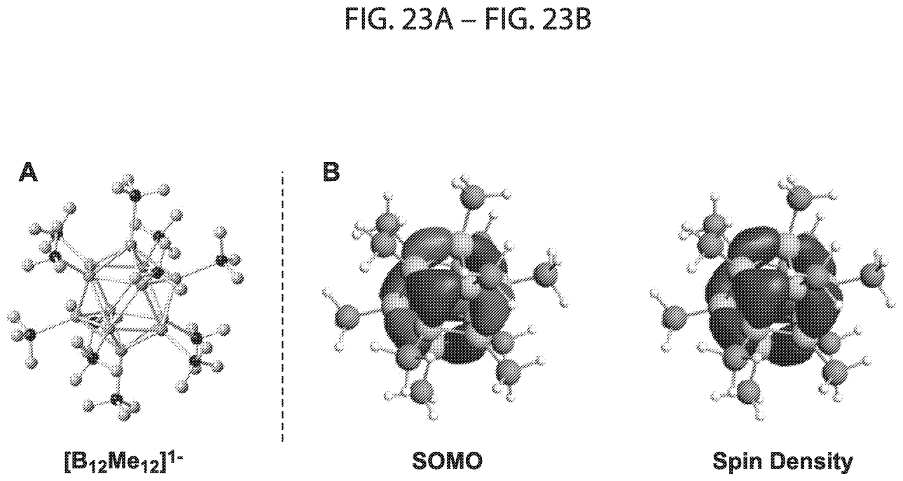

FIG. 24A-FIG. 24D depicts in accordance with various embodiments of the invention, non-limiting examples of boron clusters. FIG. 24A redox potentials of [B12(OR)12] clusters plotted versus Hammett constants (top/blue, 1− to 0; bottom/red, 2− to 1−). FIG. 24B single-crystal structure X-ray structure of cluster [e]−. FIG. 24C UV-vis spectra and photographs of the air-stable radical cluster [e]− (red solid line0 and the dianionic cluster[e]2− (blue dashed line). FIG. 24D electron paramagnetic resonance spectrum of the radical cluster [e]−. These highlight the tunable nature of the perfunctionalized boron clusters.

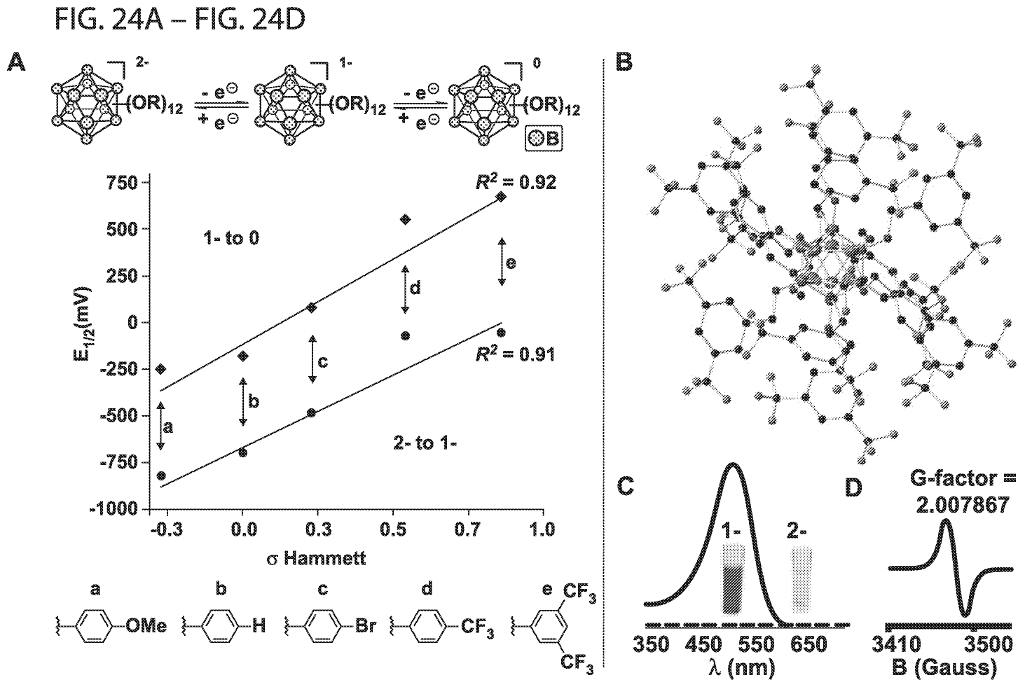

FIG. 25A depicts in accordance with various embodiments of the invention, non-limiting examples of boron clusters. FIG. 25A TD-DFT calculation describing the charge-transfer excitation pathway in hypercloso-B₁₂(OCH₂C₆F₅)₁₂.

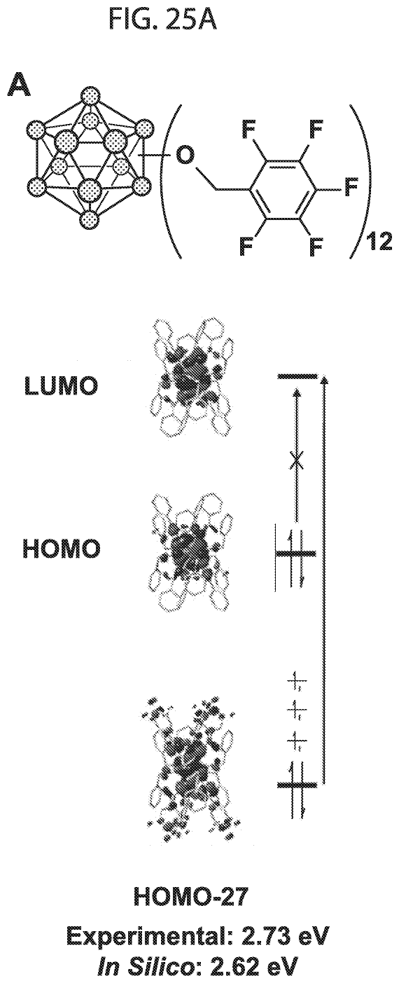

FIG. 26 depicts in accordance with various embodiments of the invention, non-limiting examples of boron clusters.

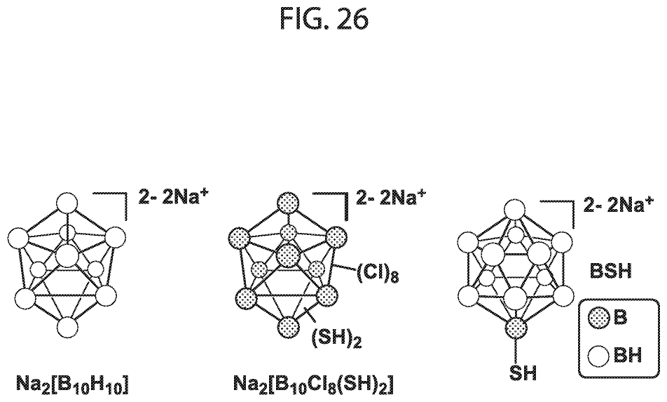

FIG. 27A-FIG. 27B depicts in accordance with various embodiments of the invention, non-limiting examples of boron clusters. FIG. 26A chemical structure. FIG. 26B 3D representation of the dodeca(nido-o-carboranyl)-substituted dodecaborate cluster.

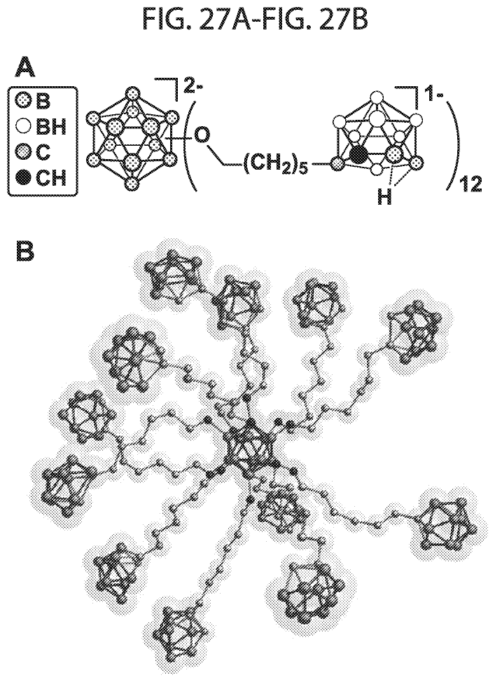

FIG. 28A-FIG. 28B depicts in accordance with various embodiments of the invention, non-limiting examples of boron clusters. FIG. 28A synthetic scheme for 1a,b from B₆H₆²⁻. FIG. 28B, IR spectra of [NBu₄][B₆H₆^{fac}], and 1a,b. The absence of stretching vibrations from ˜2100 to 2600 cm-1 for 1a,b suggests a lack of terminal B—H bonds and complete cluster substitution.

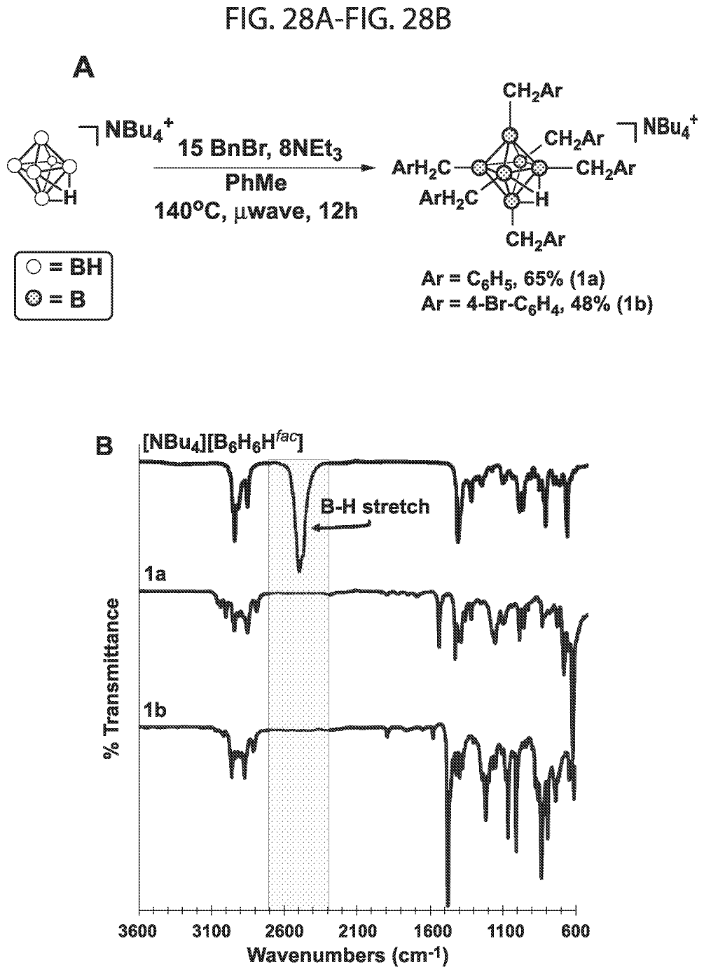

FIG. 29A-FIG. 29D depicts in accordance with various embodiments of the invention, non-limiting examples of boron clusters. Synthesis and characterization of the perfluoroaryl-perfunctionalized dodecaborate clusters and the subsequent modification with thiols. FIG. 29A perfunctionalization of 1 with rigid pentafluoroaryl-terminated linkers yields pure clusters 2 and 3, after isolation. FIG. 29B and FIG. 29C ball-and-stick and space-filling representations of the single-crystal X-ray structures of 2 and 3, respectively. Size measurements of the crystal structures reveal that 2 is 1.9 nm and 3 is 2.7 nm (lengthwise). B, pink; O, red; C, grey; F, purple. FIG. 29D ‘click’-like modification of cluster 2 with the 1-hexanethiol reagent and the corresponding ¹⁹F and ¹¹B NMR spectra associated with the transformation from the starting material 2 to the functionalized product 2a. Specifically, perfunctionalization of 2 with 1-hexanethiol results in a shift of the meta-F resonance and the complete disappearance of the para-F resonance as well as a characteristic upfield shift of the boron singlet that results from the reduction of the cluster, TBA, tetrabutylammonium.

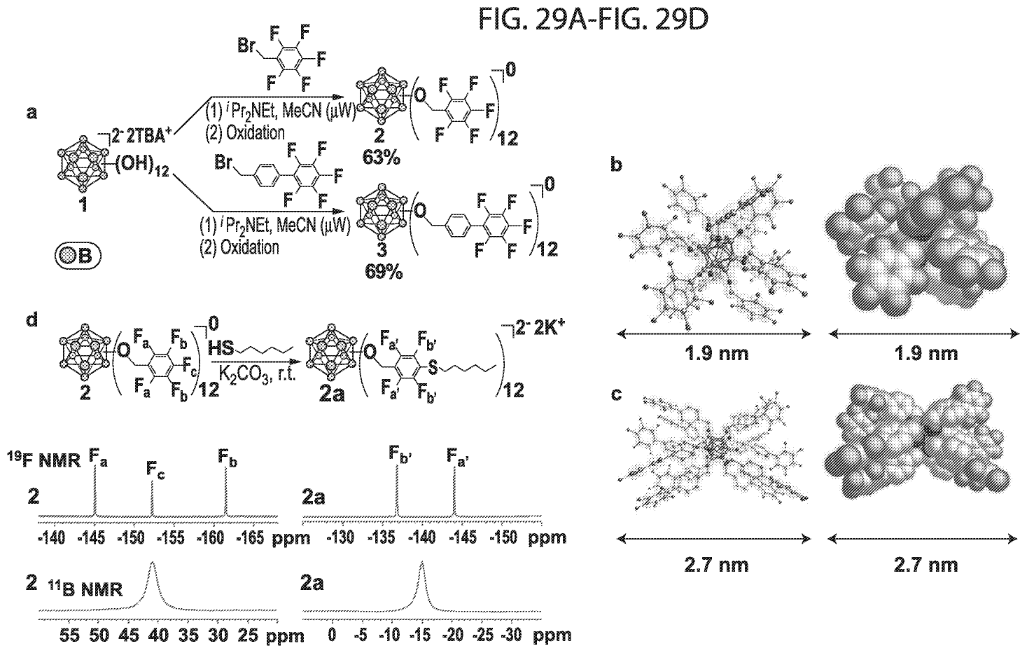

FIG. 30 depicts in accordance with various embodiments of the invention, non-limiting examples of boron clusters. Table 1, Conjugation scope for 2 and 3. *Yield determined by ¹⁹F NMR spectroscopy; [symbol] Small-scale reactions show full conversion within 1 h; [symbol] Additional 36 equiv. isopropoxytrimethylsilane (ⁱPrMe₃Si) was employed to scavenge fluoride (F⁻) by-product r.t. room temperature.

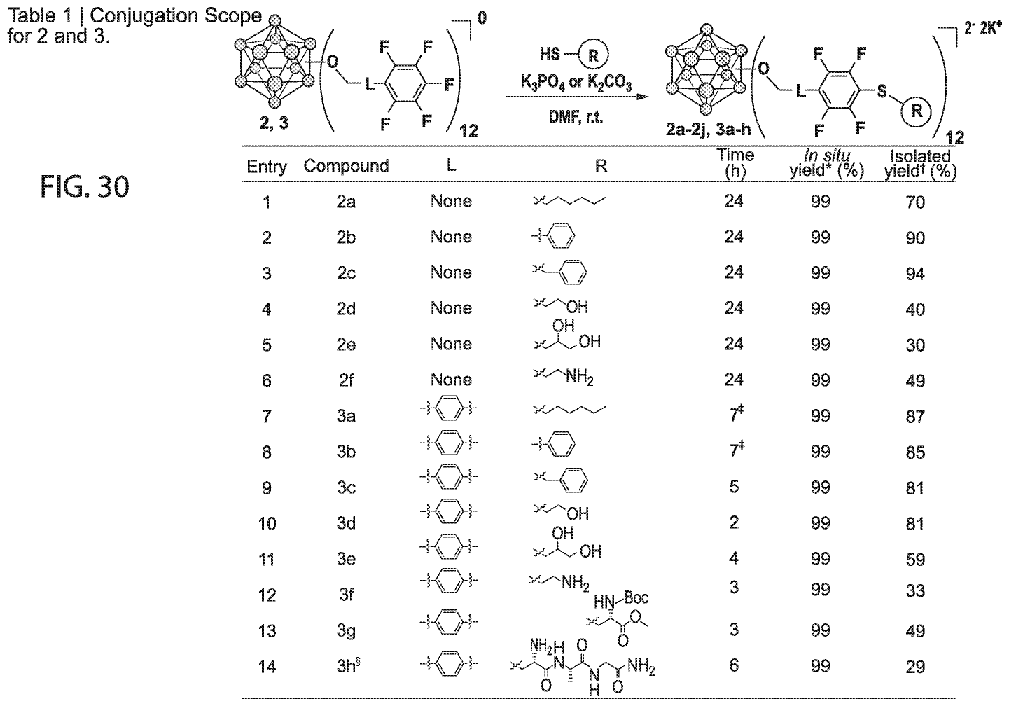

FIG. 31 depicts in accordance with various embodiments of the invention, non-limiting examples of boron clusters. Table 2, PEGylation and glycosylation of 2 and 3. *Yield determined by ¹⁹F N/R spectroscopy; ⁺Isolated yields after purification; [symbol] 2I and 3I underwent partial K⁺/Na⁺ counterion exchange during the deprotection reaction with NaOMe; Small-scale reaction shows full conversion within 5 h.

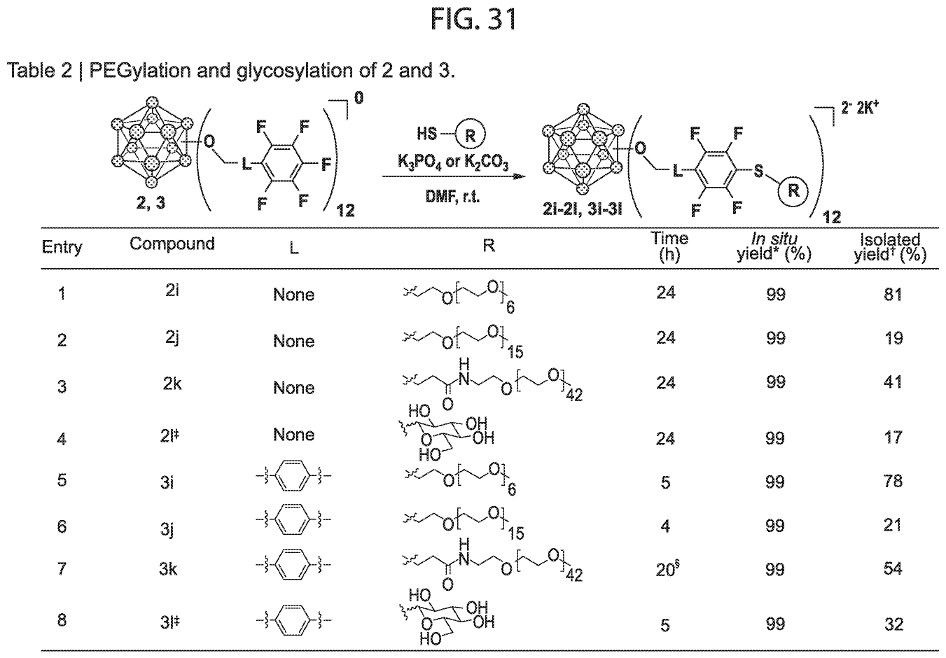

FIG. 32A-FIG. 32C depicts in accordance with various embodiments of the invention, non-limiting examples of boron clusters. FIG. 32A reaction conditions that result in the formation of R-meta-carborane regioisomers. FIG. 32B ¹¹B NMR spectra of the isolated regioisomers. Singlet resonances (no ¹¹B-¹H coupling) corresponding to the B—O-bonded vertex are labeled; all other resonances correspond to doublet resonances arising from ¹¹B-¹H couplings. FIG. 32C single-crystal X-ray structures of R1—B(n), n=2, 4, 5, 9 (ellipsoids drawn at 50% probability and H atoms omitted for clarity).

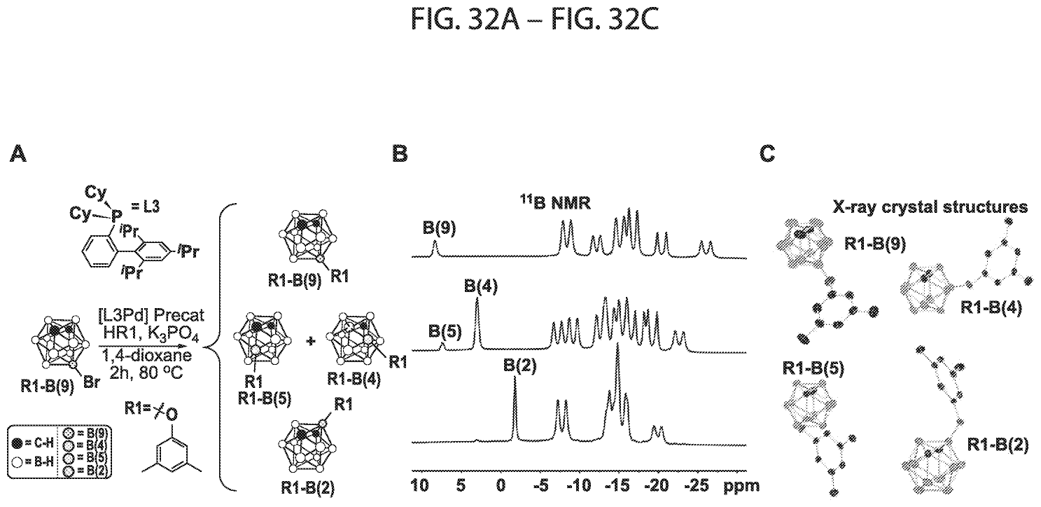

FIG. 33 depicts in accordance with various embodiments of the invention, non-limiting examples of boron clusters. Reaction conditions for forming B-functionalized meta-carborane isomers using different substrates (R1-R3) and biaryl phosphine ligands (L1-L3). Yields were obtained by GC-MS.

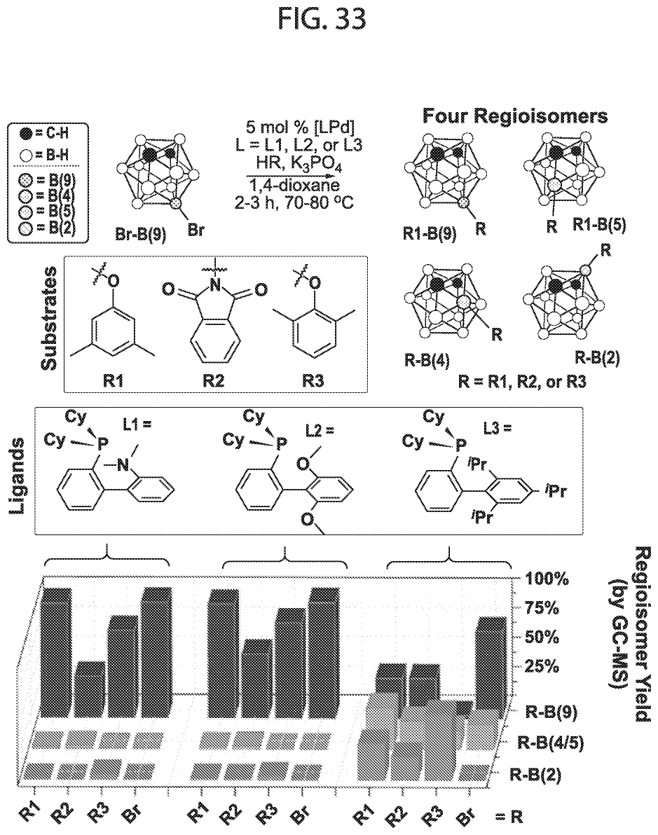

FIG. 34 depicts in accordance with various embodiments of the invention, Encapsulation of Boron Clusters in Feraheme with a Targeting Ligand—Schematic of Feraheme (top) and functionalized Feraheme (bottom) loaded with boron clusters. Measurements of boron clusters before and after functionalization of Feraheme with targeting ligands (HMC) showed no change in concentrations of boron clusters detected by ICP-OES. Hence, these measurements demonstrated that subsequent functionalization of Feraheme with HMC, does not affecting the loading of boron clusters within Feraheme.

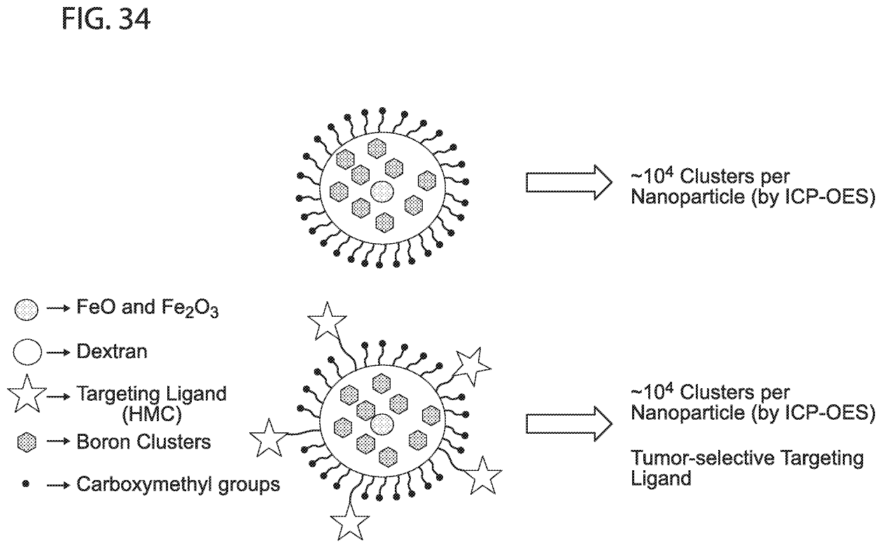

FIG. 35 depicts in accordance with various embodiments of the invention, chemical structure of HMC and HMC-Lys. Heptamethine Carbocyanine (HMC) is a near infrared fluorescent ligand that binds to OATP transporters, crosses the blood-brain barrier (BBB), and targets to tumor cells. HMC is a member of the carbocyanine family of dyes. HMC has near infrared fluourescent (750 em/800 em). HMC has strong affinity for cancer tissue, with less affinity for normal tissue. HMC has been found to cross the blood brain barrier (BBB). To increase HMC aqueous solubility and facilitate conjugation to boron clusters and nanoparticles, a lysine linker was attached to HMC to form HMC-Lys.

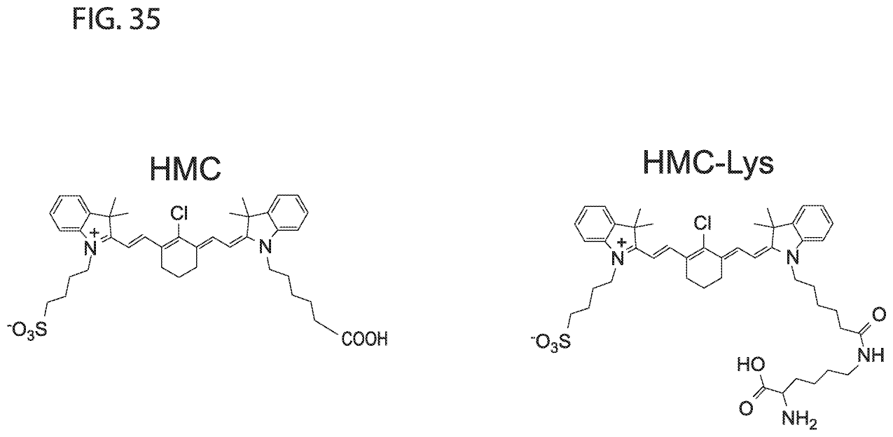

FIG. 36 depicts in accordance with various embodiments of the invention, schematic of HMC (left) and conjugates, HMC-NP (right). HMC can be utilized to fluorescently label gliomas for intraoperative surgery. In addition, conjugation of HMC via a linker to surface of Feraheme provides selective targeting and delivery of boron clusters to gliomas.

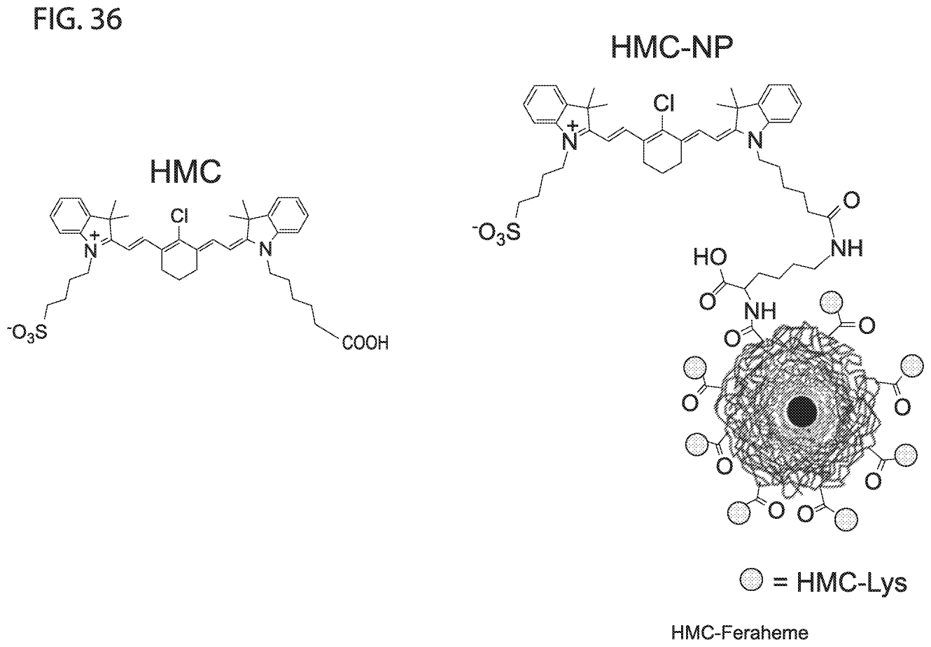

FIG. 37 depicts in accordance with various embodiments of the invention, preparation of Labeled NaBH₄. Reaction scheme to prepare NaBH₄ from B(OH)₃ which is the only commercial source for 10B labeled material.

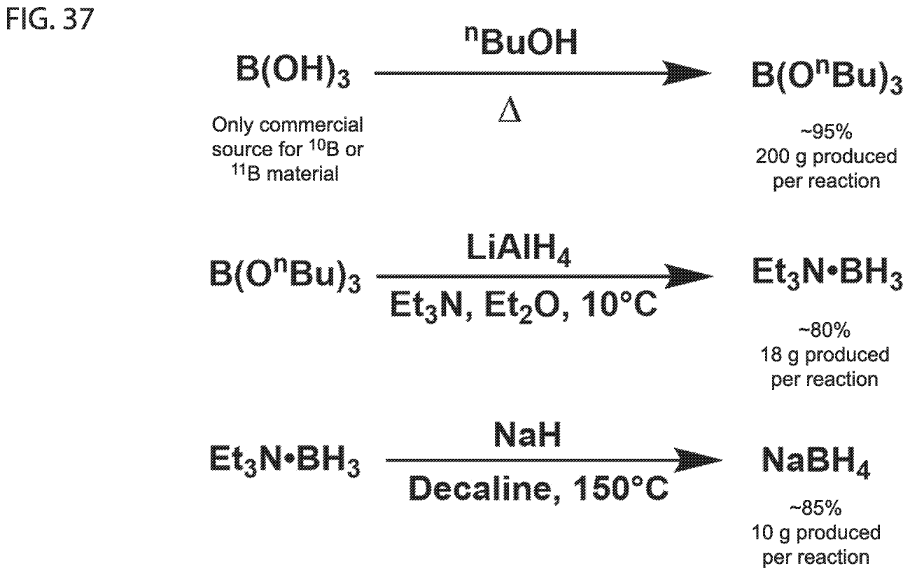

FIG. 38 depicts in accordance with various embodiments of the invention, preparation of labeled B₁₂H₁₂²⁻. Reaction procedure to produce dodecaborate from NaBH₄ as adapted from literature procedures (see Geis, V.; Guttsche, K.; Knapp, C.; Scherer, H.; Uzun, R. Dalton Trans. 2009, 2687-2694).

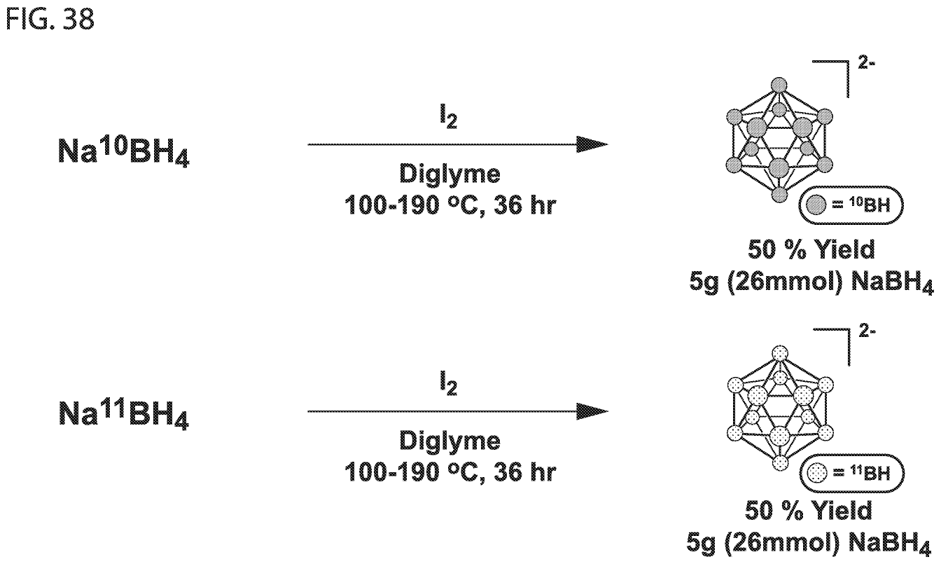

FIG. 39 depicts in accordance with various embodiments of the invention, comparison of unlabeled and ¹⁰B-labeled B₁₂H₁₂²⁻. Nuclear magnetic resonance (NMR) spectroscopy shows ¹⁰B labeled material has a higher signal to noise ratio using ¹⁰B NMR whereas unlabeled material has a higher signal to noise ratio using B N/R as ¹¹B has a higher natural abundance than ¹⁰B.

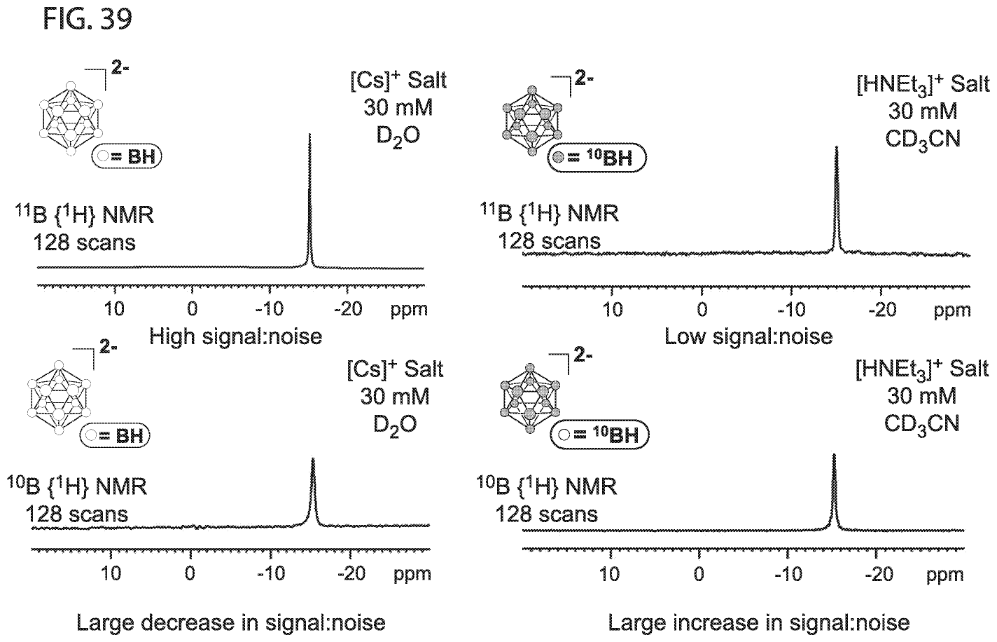

FIG. 40 depicts in accordance with various embodiments of the invention, comparison of unlabeled and ¹⁰B-labeled B₁₂H₁₂²⁻. NMR spectroscopy shows ¹⁰B labeled material has a higher signal to noise ratio using ¹⁰B N/R whereas unlabeled material has a higher signal to noise ratio using ¹¹B NMR as ¹¹B has a higher natural abundance than ¹⁰B.

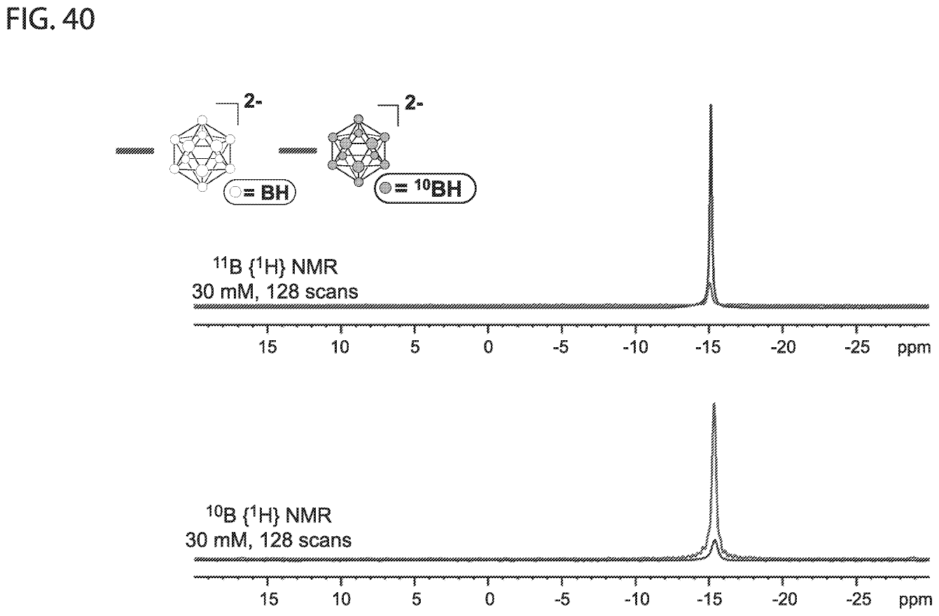

FIG. 41 depicts in accordance with various embodiments of the invention, comparison of unlabeled and ¹⁰B-labeled B₁₂H₁₂²⁻. Mass spectrometry indicates high isotopically purity for ¹⁰B labeled material based on comparisons experimental data and calculated data.

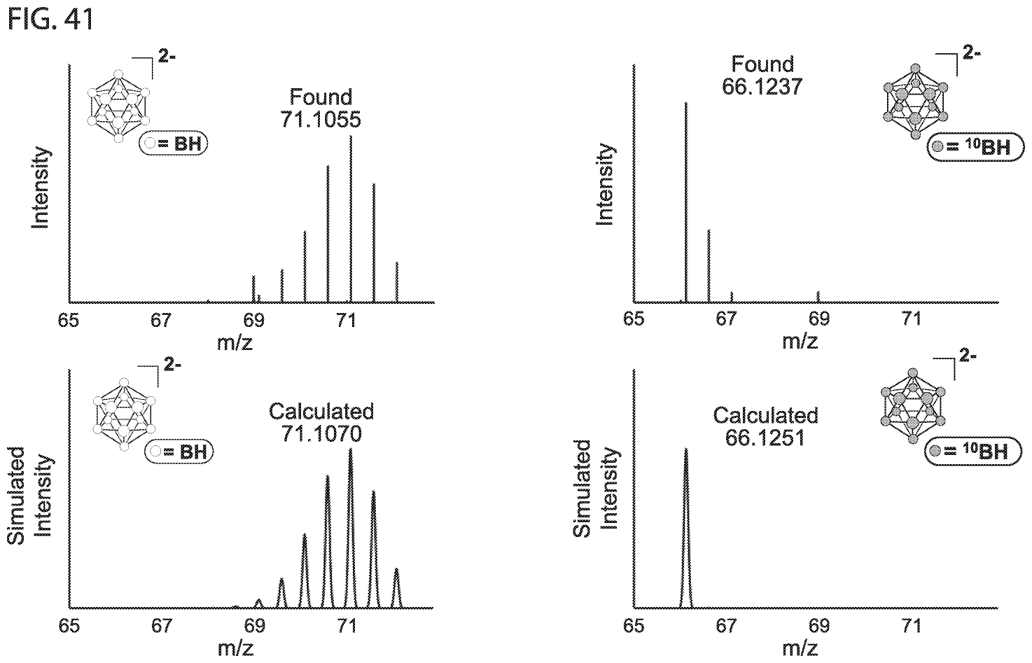

FIG. 42 depicts in accordance with various embodiments of the invention, comparison of unlabeled and ¹¹B-labeled B₁₂H₁₂²⁻. Mass spectrometry indicates high isotopically purity for ¹¹B labeled material based on comparisons between experimental data and calculated data.

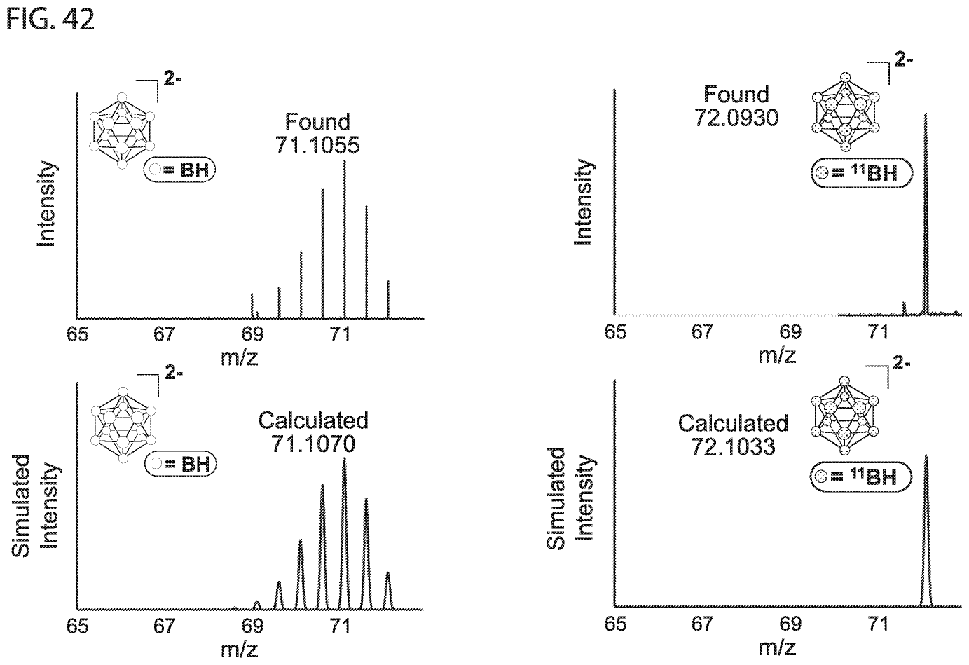

FIG. 43 depicts in accordance with various embodiments of the invention, comparison of ¹¹B-labeled B₁₂H₁₂⁻² and ¹⁰B-labeled B₁₂H₁₂²⁻. Mass spectrometry indicates a mass difference of 6 units (m/z) for the molecular ion peaks of ¹⁰B₁₂H₁₂²⁻ and ¹¹B₁₂H₁₂²⁻ as is expected.

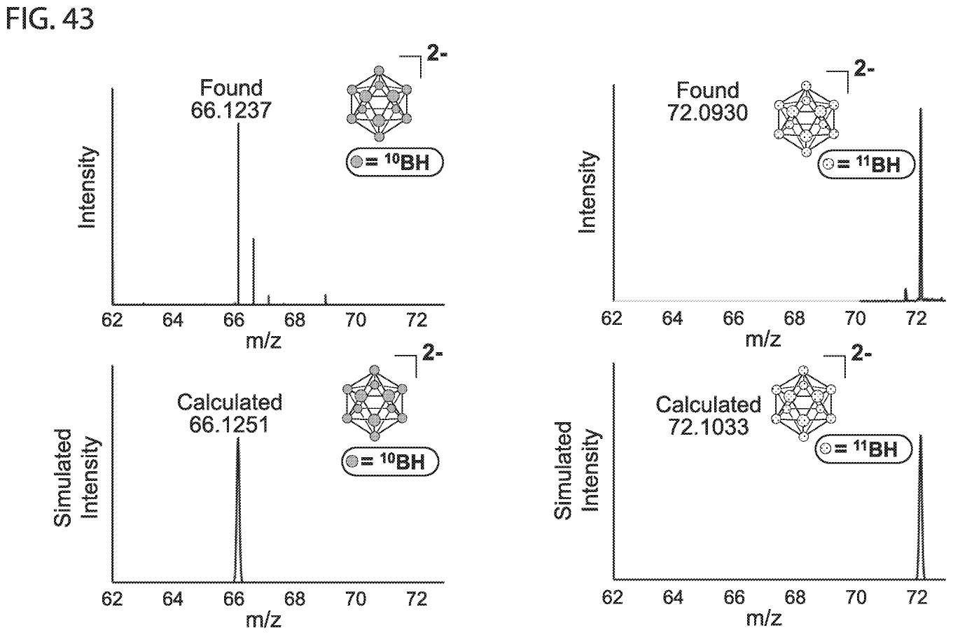

FIG. 44 depicts in accordance with various embodiments of the invention, boron cluster encapsulation into Feraheme can be monitored by N/R spectroscopy. A titration of Na₂B₁₂H₁₂ into Feraheme shows a change in chemical shift relative to free Na₂B₁₂H₁₂.

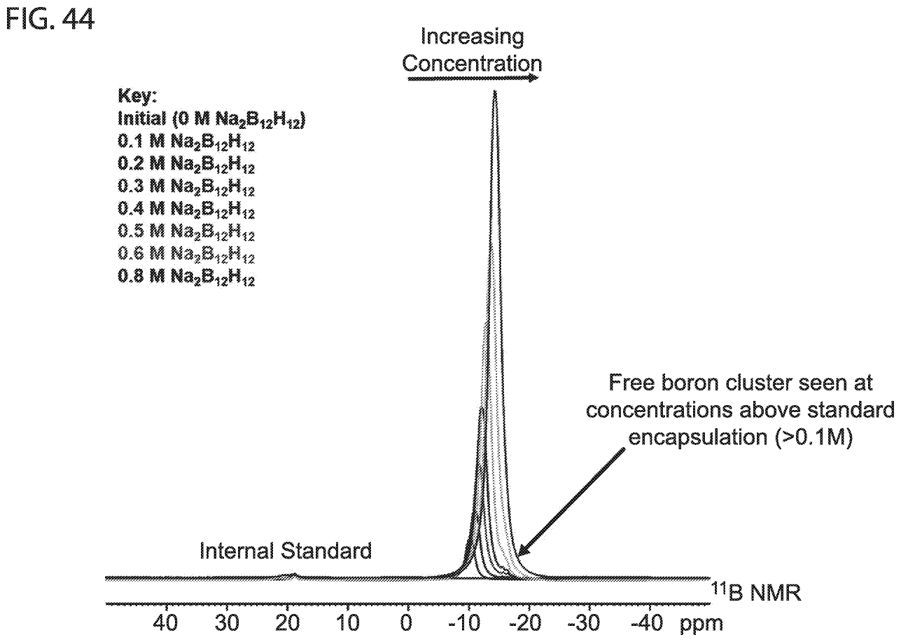

FIG. 45 depicts in accordance with various embodiments of the invention, preliminary result from measured amounts of boron in brain tumor vs healthy tissue. In vivo biodistribution of HMC-FH(BC) was conducted in intracranial mouse xenografts with glioblastoma. At 24 h after intravenous injection of HMC-FH(BC), mice were sacrificed and the brains and tumors were excised, followed by digestion with acid to dissolve organic matters. Concentrations of boron in brains and tumors were measured by ICP-OES. Remarkably, 50% injected dose of boron per gram of tissue (% ID [B]/g tissue) was reported for mouse injected with a dose of HMC-FH(BC) at 7 mg [Fe]/kg. In comparison, a injected dose of HMC-FH(BC) at 4 mg [Fe]/kg, the measured dose was reported to be 14% ID [B]/g tissue. Likewise, no boron was detected in mouse not injected with HMC-FH(BC).

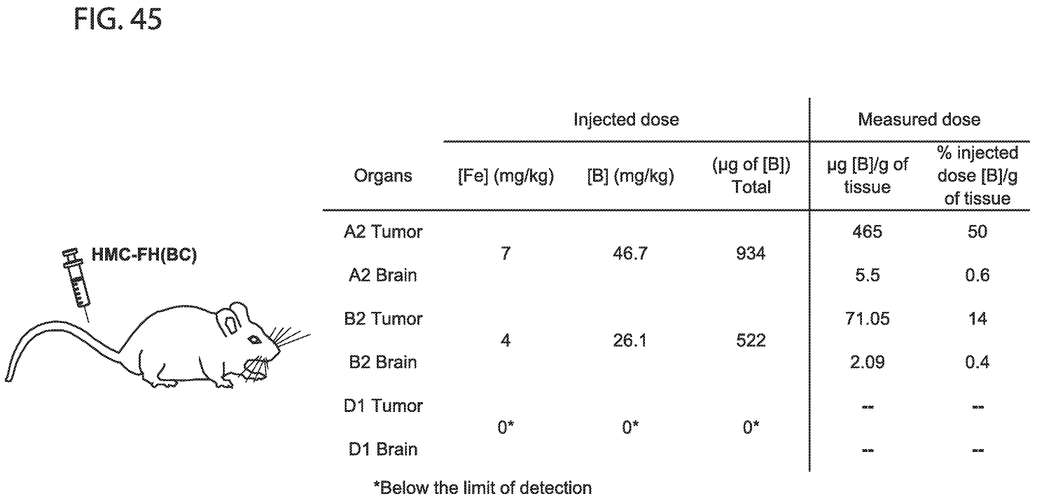

FIG. 46 depicts in accordance with various embodiments of the invention, an HMC-FH platform technology can be used to facilitate the pre-operative MRI and intraoperative fluorescent assessment of tumor margins. The same nanoparticle technology can be used to deliver boron clusters to tumors via HMC-FH(boron cluster), where FH is Feraheme and boron cluster is encapsulated within the carboxymethyl dextran coating on FH.

FIG. 47 depicts in accordance with various embodiments of the invention, Heptamethine cyanine (HMC) dyes and conjugates. The near infrared dye and OATP-targeting ligand HMC can be conjugated with a lysine linker to yield HMC-Lys, which can then be conjugated to carboxylic acid groups on Feraheme (FH). The HMC dye binds to the OATP receptor in cancer cells. HMC has near infrared fluorescence (ex/em 750/800). Therefore, an HMC-FH nanoprobe will target cancer cells via the OATP receptor, labeling the tumor with iron oxide for MR Imaging and fluorescent for intraoperative surgery. When a boron cluster is encapsulated in the HMC-FH nanocarrier, the resulting HMC-FH(boron cluster) will deliver the boron cluster to tumor, causing tumor regression and improved survival.

FIG. 48 depicts in accordance with various embodiments of the invention, Low molecular weight PSMA-targeting glutamate urea based probe. F—N—[N—[(S)-1,3-dicarboxypropyl]carbamoyl]-4-fluorobenzyl-1-cysteine (18F-DCFBC).

FIG. 49 depicts in accordance with various embodiments of the invention, Theranostics HM-Feraheme (Boron cluster) nanoparticle. A boron cluster, is encapsulated within the carboxymethyl dextran coating of either Glu-Feraheme or Fol-Feraheme. The resulting nanoparticle with dual therapeutic and imaging can deliver boron clusters to cancer cells via PSMA, while being able to visualize boron cluster-nanoparticle localization in tissue by imaging methods.

FIG. 50 depicts in accordance with various embodiments of the invention, Angiopep-Feraheme nanoparticles. The iron oxide core (IO) is surrounded by a polymeric coating such as carboxymethyl dextran that can encapsulate a boron cluster and where carboxylic acid groups are conjugated to Angiopep to facilitate crossing of the BBB and uptake by glioblastoma cells.

FIG. 51 depicts in accordance with various embodiments of the invention, Conjugation of Angiopep-Cysteine (TFFYGGSRGKRNNFKTEEYC) (SEQ ID NO: 1) onto Feraheme carboxylic acid groups. A Maleimide-PEG-Amine linker was first conjugated to the carboxylic acid group on Feraheme to yield a Maleimide-PEG-Feraheme before reaction with the Angiopep-Cysteine peptide.

FIG. 52 depicts in accordance with various embodiments of the invention, Theranostics HM-Feraheme (Boron cluster) nanoparticle. A boron cluster, is encapsulated within the carboxymethyl dextran coating of HM-Feraheme. The resulting nanoparticle with dual therapeutic and imaging (fluorescent and MRI) can deliver boron clusters to cancer cells via the OATP receptor, while being able to visualize boron cluster-nanoparticle localization in tissue by imaging methods.

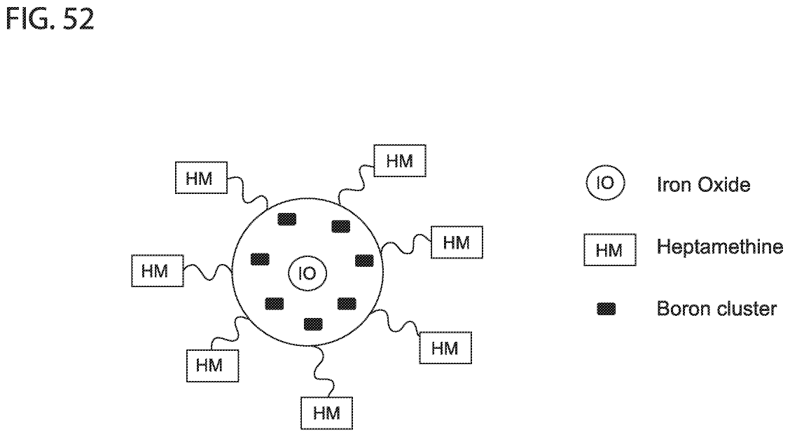

FIG. 53 depicts in accordance with various embodiments of the invention, Conjugation of Heptamethine to Feraheme carboxylic acid groups. A heptamethine-lysine conjugate (HM-Lys-NH2) was conjugated to the available carboxylic acid groups on the surface of Feraheme using EDC/NHS chemistry.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7th ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cellfusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

“Optional” or “optionally” means that the subsequently described circumstance may or may not occur, so that the description includes instances where the circumstance occurs and instances where it does not.

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amidine, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring. In some cases, two or more substituents, together with the carbon(s) to which they are attached to, can form one or more rings.

The terms “substituted” and “functionalized” are used interchangeably herein.

The terms “unsubstituted” and “unfunctionalized” are used interchangeably herein.

Substituents may be protected as necessary and any of the protecting groups commonly used in the art may be employed. Non-limiting examples of protecting groups may be found, for example, in Greene and Wuts, Protective Groups in Organic Synthesis, 44^{th}. Ed., Wiley & Sons, 2006.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In some embodiments, a straight chain or branched chain alkyl has 30 or fewer carbon atoms in its backbone (e.g., C₁-C₃₀ for straight chains, C₃-C₃₀ for branched chains), and in some embodiments 20 or fewer. Likewise, in some embodiments cycloalkyls have from 3-10 carbon atoms in their ring structure, and some embodiments have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, in some embodiments from one to six carbon atoms in its backbone structure. Likewise, “lower alkenyl” and “lower alkynyl” have similar chain lengths. Throughout the application, in some embodiments alkyl groups are lower alkyls. In some embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkenyl, and alkynyl” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Rₐ and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂—), ethylidene (═CHCH₃), isopropylidene (═C(—CH₃)₂), propylidene (═CHCH₂CH₃), allylidene (═CH—CH═CH₂—), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “carbocyclyl” as used either alone or in combination with another radical, means a mono- bi- or tricyclic ring structure consisting of 3 to 14 carbon atoms. In some embodiments, one or more of the hydrogen atoms of a carbocyclyl may be optionally substituted by a substituent.

The term “carbocycle” refers to fully saturated ring systems and saturated ring systems and partially saturated ring systems and aromatic ring systems and non-aromatic ring systems and unsaturated ring systems and partially unsaturated ring systems. The term “carbocycle” encompasses monocyclic, bicyclic, polycyclic, spirocyclic, fused, bridged, or linked ring systems. In some embodiments, one or more of the hydrogen atoms of a carbocycle may be optionally substituted by a substituent. In some embodiments the carbocycle optionally comprises one or more heteroatoms. In some embodiments the heteroatoms are selected from N, O, S, or P.

The terms “cyclic” “cyclic group” and “ring” or “rings” means carbocycles, which can be fully saturated, saturated, partially saturated, unsaturated, partially unsaturated non-aromatic or aromatic that may or may not be substituted and which optionally can comprise one or more heteroatoms. In some embodiments the heteroatoms are selected from N, O, S, or P. In some embodiments, one or more of the hydrogen atoms of a ring may be optionally substituted by a substituent. In some embodiments, the ring or rings may be monocyclic, bicyclic, polycyclic, spirocyclic, fused, bridged, or linked.

The term “spiro-cycloalkyl” (spiro) means spirocyclic rings where the ring is linked to the molecule through a carbon atom, and wherein the resulting carbocycle is formed by alkylene groups. The term “spiro-C₃-C₈-cycloalkyl” (spiro) means 3-8 membered, spirocyclic rings where the ring is linked to the molecule through a carbon atom, and wherein the resulting 3-8 membered carbocycle is formed by alkylene groups with 2 to 7 carbon atoms. The term “spiro-C₅-cycloalkyl” (spiro) means 5 membered, spirocyclic rings where the ring is linked to the molecule through a carbon atom, wherein the resulting 5 membered carbocycle is formed by an alkylene group with 4 carbon atoms.

The term “spiro-cycloalkenyl” (spiro) means spirocyclic rings where the ring is linked to the molecule through a carbon atom, and wherein the resulting carbocycle is formed by alkenylene groups. The term “spiro-C₃-C₈-cycloalkenyl” (spiro) means 3-8 membered, spirocyclic rings where the ring is linked to the molecule through a carbon atom, wherein the resulting 3-8 membered carbocycle is formed by alkenylene groups with 2 to 7 carbon atoms. The term “spiro-C₅-cycloalkenyl” (spiro) means 5 membered, spirocyclic rings where the ring is linked to the molecule through a carbon atom, wherein the resulting 5 membered carbocycle is formed by alkenylene groups with 4 carbon atoms.

The term “spiro-heterocyclyl” (spiro) means saturated or unsaturated spirocyclic rings, which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₃-C₈-heterocyclyl” (spiro) means 3-8 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₅-heterocyclyl” (spiro) means 5 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N.

In some embodiments, one or more of the hydrogen atoms of a spirocyclic ring may be optionally substituted by a substituent. In some embodiments, one or more hydrogen atoms of a spiro-cycloalkyl may be optionally substituted by a substituent. In some embodiments, one or more hydrogen atoms of a spiro-C₃-C₈-cycloalkyl may be optionally substituted by a substituent. In some embodiments, one or more hydrogen atoms of a spiro-C₅-cycloalkyl may be optionally substituted by a substituent. In some embodiments, one or more hydrogen atoms of a spiro-cycloalkenyl may be optionally substituted by a substituent. In some embodiments, one or more hydrogen atoms of a spiro-C₃-C₈-cycloalkenyl may be optionally substituted by a substituent. In some embodiments, one or more hydrogen atoms of a spiro-C₅-cycloalkenyl may be optionally substituted by a substituent. In some embodiments, one or more hydrogen atoms of a spiro-heterocycyl may be optionally substituted by a substituent. In some embodiments, one or more hydrogen atoms of a spiro-C₃-C₈-heterocycyl may be optionally substituted by a substituent. In some embodiments, one or more hydrogen atoms of a spiro-C₅-heterocycyl may be optionally substituted by a substituent.

As used herein, the term “carbonyl” means the radical C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺+(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term “oxo” means the substituent ═O.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, NHCH₃, —N(—CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(—CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(—CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino” —NH-(alkyl)-(cycloalkyl), such as —NHCH₂-cyclohexyl, and the like.

The term “sulfonato” means —SO₃.

The term “PEGyl” refers to a polyethylene chain with repeated moiety of (—CH₂—CH₂—O—)ₙ. n is ranging from 2 to 20. The remote end of the PEG may be optionally functionalized with amino, carboxylate, sulfonate, alkyne, sulfohydryl, hydroxyl, or any other functional group.

“Electron withdrawing group” or EWG refers to functional groups that remove electron density from the ring by making it less nucleophilic. This class can be recognized by the atom adjacent to the n system having several bonds to more electronegative atoms or the presence of a formal charge. Non-limiting examples of these groups include halogens, aldehydes, ketones, esters, carboxylic acids, acid chlorides, nitriles, nitrosos, and sulfonic acids.

“Electron donating group” or EDG refers to functional groups that add electron density to the ring by making it more nucleophilic. This class can be recognized by lone pairs on the atom adjacent to the n system. Non-limiting examples of these groups include alkyl, alkenyl, alkynyl, amides, ethers, alkoxides, alcohols, and amines.

Some commonly used abbreviations are: Me is methyl (—CH₃), Et is ethyl (—CH₂—CH₃), Ph is phenyl (—C₆H₅), t-Bu is tert-butyl (—C(—CH₃)₃, n-Pr is n-propyl (—CH₂—CH₂—CH₃), Bn is benzyl (—CH₂—C₆H₅).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

As used herein, the terms “heptamethine cyanine (HMC)”, “heptamethine carbocyanine (HMC)” and “HMC” have the same meaning and refer to the following compound:

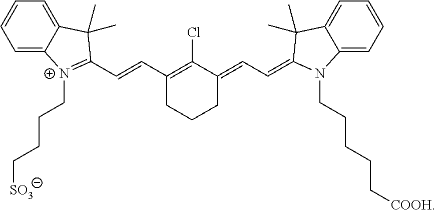

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

Synthetic Preparation. In various embodiments, compounds, compositions, formulations, articles of manufacture, reagents, products, etc. (e.g., compositions, polymers, copolymers, nanovehicles, nanoparticles, boron clusters, etc.) of the present invention as disclosed herein may be synthesized using any synthetic method available to one of skill in the art. In various embodiments, the compounds, compositions, formulations, articles of manufacture, reagents, products, etc. (e.g., compositions, polymers, copolymers, nanovehicles, nanoparticles, boron clusters, etc.) of the present invention disclosed herein can be prepared in a variety of ways known to one skilled in the art of organic synthesis, inorganic synthesis, and/or organometallic synthesis and in analogy with the exemplary compounds, compositions, formulations, articles of manufacture, reagents, products, etc. whose synthesis is described herein. The starting materials used in preparing these compounds, compositions, formulations, articles of manufacture, reagents, products, etc. may be commercially available or prepared by known methods. Preparation of compounds, can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, which is incorporated herein by reference in its entirety.

Non-limiting examples of synthetic methods used to prepare various embodiments of compounds, compositions, formulations, articles of manufacture, reagents, products, etc. (e.g., compositions, polymers, copolymers, nanovehicles, nanoparticles, boron clusters, etc.) of the invention are disclosed in the Examples section herein. The reactions of the processes described herein can be carried out in suitable solvents which can be readily selected by one of skill in the art of organic synthesis, inorganic synthesis, and/or organometallic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

As used herein, the terms “treat,” “treatment,”, “treating,” or “amelioration” when used in reference to a symptom, disease, disorder or disease condition, refer to both therapeutic treatment and prophylactic or preventative measures, wherein the object is to reverse, alleviate, ameliorate, inhibit, lessen, slow down or stop the progression or severity of a symptom, disease, disorder, or disease condition. The term “treating” includes reducing or alleviating at least one adverse effect or symptom of a disease condition, disease, or disorder. Treatment is generally “effective” if one or more symptoms or clinical markers are reduced. Alternatively, treatment is “effective” if the progression of a symptom, disease, disorder or disease condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the disease condition, disease, or disorder even if the treatment is ultimately unsuccessful. Those in need of treatment include those already with the symptom, disease condition, disease, disorder as well as those prone to have the symptom, disease condition, disease, or disorder, or those in whom the symptom, disease condition, disease, or disorder is to be prevented. Treatment also includes a decrease in mortality or an increase in the lifespan of a subject as compared to one not receiving the treatment.

The term “preventative treatment” means maintaining or improving a healthy state or non-diseased state of a healthy subject or subject that does not have a symptom, disease, disorder, or disease condition. The term “preventative treatment” also means to prevent or to slow the appearance of symptoms associated with a disease condition, disease, or disorder. The term “preventative treatment” also means to prevent or slow a subject from obtaining a symptom, disease condition, disease, or disorder.

“Beneficial results” or “desired results” may include, but are in no way limited to, lessening or alleviating the severity of the symptom, disease, disorder, or disease condition; preventing the symptom, disease, disorder, or disease condition from worsening; curing the symptom, disease, disorder, or disease condition; preventing the symptom, disease, disorder, or disease condition from developing; lowering the chances of a patient developing the symptom, disease, disorder, or disease condition; decreasing morbidity and mortality, and prolonging a patient's life or life expectancy. As non-limiting examples, “beneficial results” or “desired results” may be alleviation of one or more symptom(s); diminishment of extent of the deficit; stabilized (i.e., not worsening) state of a symptom, disease, disorder, or disease condition; delay or slowing of a symptom, disease, disorder, or disease condition; and amelioration or palliation of symptoms associated with a symptom, disease, disorder, or disease condition.

The term “disease” refers to an abnormal condition affecting the body of an organism. For example, the disease or abnormal condition may result from a pathophysiological response to external or internal factors.

The term “disorder” refers to a functional abnormality or disturbance. For example, a disorder may be a disruption of the disease to the normal or regular functions in the body or a part of the body.

The term “disease condition” refers to an abnormal state of health that interferes with the usual activities of feeling or wellbeing.

The term “normal condition” or “healthy condition” refers to a normal state of health.

The term “healthy state” or “normal state” means that the state of the subject (e.g., biological state or health state, etc.) is not abnormal or does not comprise a disease, disorder, or disease condition.

“Diseases”, “disorders” and “disease conditions,” as used herein may include, but are in no way limited to any form of a cancer.

In various embodiments, the disease is at least one cancer. In various embodiments, the disorder is at least one cancer. In various embodiments, the disease condition is at least one cancer.

Examples of cancer include but are not limited to breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; cervical cancers such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; acute myeloid leukemia (AML), preferably acute promyleocytic leukemia in peripheral blood; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's sarcoma; Ewing sarcoma; central nervous system cancers such as primary brain tumor, which includes gliomas (astrocytoma, anaplastic astrocytoma, or glioblastoma multiforme (GBM)), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas; oral cavity and oropharyngeal cancer; stomach cancer such as lymphomas, gastric stromal tumors, and carcinoid tumors; testicular cancer such as germ cell tumors (GCTs), which include seminomas and nonseminomas; and gonadal stromal tumors, which include Leydig cell tumors and Sertoli cell tumors; head cancer; neck cancer; throat cancer; and thymus cancer, such as to thymomas, thymic carcinomas, Hodgkin disease, non-Hodgkin lymphomas carcinoids or carcinoid tumors. Also, the methods can be used to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In some embodiments, the cancer is metastasized. In some embodiments, the cancer is glioma. In some embodiments, the glioma is selected from the group consisting of astrocytoma, anaplastic astrocytoma, glioblastoma multiforme (GBM), oligodendroglioma and combinations thereof.

A “healthy subject” or “normal subject” is a subject that does not have a disease, disease condition, or disorder.

The term “unhealthy subject” or “abnormal subject” is a subject that does have a disease, disease condition, or disorder.

As used herein, the term “administering,” refers to the placement of a compound or an agent (e.g., nanovehicle of the present invention, a nanoparticle of the present invention, boron cluster, drug, probe, or pharmaceutical composition) or a treatment as disclosed herein into a subject by a method or route which results in at least partial localization of the compound, agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the compound, agent or treatment can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the compounds, agent or treatment can be in the form of aerosol, lotion, cream, gel, ointment, suspensions, solutions or emulsions. In accordance with the present invention, “administering” can be self-administering. For example, it is considered as “administering” that a subject consumes a composition, compound, agent or treatment as disclosed herein (e.g., a nanovehicle of the present invention, nanoparticle of the present invention, boron cluster, drug, probe, or pharmaceutical composition).

As used herein, an “effective amount” is that amount effective to bring about the physiological change desired in the subject or sample to which a compound or agent (e.g., nanovehicle of the present invention, nanoparticle of the present invention, boron cluster, drug, probe, or pharmaceutical composition) is administered. The term “therapeutically effective amount” as used herein, means that amount of a compound or agent (e.g., nanovehicle of the present invention, nanoparticle of the present invention, boron cluster, drug, probe, or pharmaceutical composition), alone or in combination, or in combination with another compound or agent according to an embodiment of the invention, that elicits the biological or medicinal response in a subject or sample that is being sought by a researcher, veterinarian, medical doctor, or other clinician, which includes alleviation of the symptoms of the disease, disorder, or disease condition being treated. For example, an effective amount of the boron cluster or drug is that amount sufficient to treat a pathological condition (e.g., a disease, disorder, or disease condition) in the subject or sample to which the boron cluster and/or drug is administered. For example, in the case of cancer, the therapeutically effective amount of the boron cluster and/or drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the boron cluster and/or drug may prevent growth and/or kill existing cancer cells, it may be cytostatic and/or cytotoxic. For cancer therapy, efficacy can, for example, be measured by assessing the time to disease progression (TTP) and/or determining the response rate (RR).

“Diagnostic” means identifying the presence or nature of a symptom, disease, disease condition, or disorder and includes identifying patients who are at risk of developing a specific symptom, disease condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a symptom, a disease condition, a disease, or a disorder, it suffices if the method provides a positive indication that aids in diagnosis.

By “at risk of” is intended to mean at increased risk of, compared to a normal subject, or compared to a control group, e.g. a patient population, or a reference. Thus a subject carrying a particular marker may have an increased risk for a specific symptom, disease condition, disease or disorder, and be identified as needing further testing. “Increased risk” or “elevated risk” mean any statistically significant increase in the probability, e.g., that the subject has the symptom, disease, disease condition, or disorder. In some embodiments the risk is increased by at least 10% over the control group or reference with which the comparison is being made. In some embodiments, the risk is increased by at least 20% over the control group or reference with which the comparison is being made. In some embodiments, the risk is increased by at least 50% over the control group or reference with which the comparison is being made.

In some embodiments, the reference is selected from: (i) a control subject or a sample from the control subject, wherein the control subject does not have the disease, disorder, or disease condition; (ii) a control subject or a sample from the control subject, wherein the control subject has the disease, disorder, or disease condition; (iii) the subject or a sample from the subject that was obtained from the subject at an earlier point in time; (iv) a healthy subject or a sample from the healthy subject; an (v) the subject or a sample from the subject after the subject was treated for the disease, disorder, or disease condition.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting a symptom, detecting a disease condition, detecting a disease, or detecting a disorder (e.g. when positive assay results are obtained). In some embodiments, the terms “detection”, “detecting” and the like, may be used in the context of detecting a nanoparticle of the present invention bound to a tissue (e.g., a tissue, a cell, a cancerous tissue, cancer tissue, cancer cell, tumor, tumor cell, or tumor tissue).

The term “diagnosis,” or “dx,” refers to the identification of the nature and cause of a certain phenomenon. As used herein, a diagnosis typically refers to a medical diagnosis, which is the process of determining which disease, disorder or disease condition explains a symptoms and signs. A diagnostic procedure, often a diagnostic test or assay, can be used to provide a diagnosis. A diagnosis can comprise detecting the presence of a disease, disorder, or disease condition, or the risk of getting a disease, disorder, or disease condition.

The term “prognosis,” or “px,” as used herein refers to predicting the likely outcome of a current standing. For example, a prognosis can include the expected duration and course of a symptom, disease, disease condition, or disorder, such as progressive decline or expected recovery.

The term “theranosis,” or “tx” as used herein refers to a diagnosis or prognosis used in the context of a medical treatment. For example, theranostics can include diagnostic testing used for selecting appropriate and optimal therapies (or the inverse) based on the context of genetic content or other molecular or cellular analysis. Theranostics includes pharmacogenomics, personalized and precision medicine.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′.sub.2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue; a tissue print; a fingerprint; skin or hair; fragments and derivatives thereof. Exemplary samples or biological samples include, but are not limited to, cheek swab; mucus; whole blood, blood, serum; plasma; urine; saliva; semen; lymph; fecal extract; sputum; other body fluid or biofluid; cell sample; a tumor sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. In some embodiments, a sample or biological sample is a tissue or tissue sample. In some embodiments, a sample or biological sample can be a tumor cell sample, e.g. the sample can comprise cancerous cells, cells from a tumor, and/or a tumor biopsy. In some embodiments, a sample or biological sample can comprise one or more cells from the subject. In some embodiments, a sample or biological sample can comprise one or more tissue samples from the subject.

In some embodiments, a sample can comprise one or more cells from the subject. In some embodiments, the sample can comprise one or more tissues from the subject. In some embodiments, a sample is a cell or cell sample. In some embodiments, a sample is a tissue or tissue sample. In some embodiments, the sample is a tumor, tumor tissue, or tumor cell. In some embodiments, the sample is a cancer cell or cancer tissue. In some embodiments, a sample can be a tumor cell sample, e.g. the sample can comprise cancerous cells, cancer cells, cells from a tumor, and/or a tumor biopsy. In some embodiments, the tissue is a cancer tissue. In some embodiments, the tissue is a tumor tissue. In some embodiments, the cell is a cancer cell. In some embodiments, the cell is a tumor cell.

Non-limiting examples of samples or biological samples include, cheek swab; mucus; whole blood, blood, serum; plasma; blood products, urine; saliva; semen; lymph; fecal extract; sputum; other body fluid or biofluid; cell sample; tissue sample; tissue extract; tissue biopsy etc.

In some embodiments, samples or biological samples comprise blood products, including whole blood, blood, plasma and/or serum. In some embodiments, samples or biological samples comprise derivatives of blood products, including whole blood, blood, plasma and/or serum. In some embodiments, the sample is a biological sample. In some embodiments, the sample is whole blood. In some embodiments, the sample is blood. In some embodiments, the sample is plasma. In some embodiments, the sample is serum.

In some embodiments, the sample is a tissue sample. In some embodiments, the sample is a tissue extract. In some embodiments the sample is a biopsy sample. In some embodiments the sample is a biopsy specimen.

The terms “body fluid” or “bodily fluids” are liquids originating from inside the bodies of organisms. Bodily fluids include amniotic fluid, aqueous humour, vitreous humour, bile, whole blood, blood (e.g., serum, plasma), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. Immunoglobulin G (IgG), the most abundant antibody subclass, may be found in all body fluids. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device. Additional sample collection procedures and devices include but are not limited to: a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) samples can be stored and mailed, and an assay can be performed remotely.

As used herein, the term “amino acid” refers to naturally occurring and synthetic amino acids, as well as amino acid analogs and amino acid mimetics that operate in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refer to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that operates in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide.” A protein contains at least one polypeptide. Short polypeptidesare sometimes referred to as “peptides.”

The term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chern. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn eta/., (1990) Pp. 969-970 in Proc. 11th American Peptide Symposium, ESCOM Science Publishers, The Netherlands, and the like)).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In some embodiments, the subject is a human.

The terms “subject”, “patient” or “individual” generally refer to a human, although the methods of the invention are not limited to humans, and should be useful in other animals (e.g. birds, reptiles, amphibians, mammals), particularly in mammals, since albumin is homologous among species.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a disease, disorder, or disease condition in need of treatment (e.g., a cancer) or one or more complications related to the disease, disorder, or disease condition, and optionally, have already undergone treatment for the disease, disorder, or disease condition or the one or more complications related to the disease, disorder, or disease condition. Alternatively, a subject can also be one who has not been previously diagnosed as having a disease, disorder, or disease condition or one or more complications related to the disease, disorder, or disease condition. For example, a subject can be one who exhibits one or more risk factors for a disease, disorder, or disease condition or one or more complications related to the disease, disorder, or disease condition or a subject who does not exhibit risk factors. For example, a subject can be one who exhibits one or more symptoms for a disease, disorder, or disease condition or one or more complications related to the disease, disorder, or disease condition or a subject who does not exhibit symptoms. A “subject in need” of diagnosis or treatment for a particular disease, disorder, or disease condition can be a subject suspected of having that disease, disorder, or disease condition, diagnosed as having that disease, disorder, or disease condition, already treated or being treated for that disease, disorder, or disease condition, not treated for that disease, disorder, or disease condition, or at risk of developing that disease, disorder, or disease condition.

In some embodiments, the subject is at risk of developing cancer. In some embodiments, the subject has cancer. In some embodiments, the subject has been diagnosed with cancer. In some embodiments, the subject is at risk of developing cancer. In some embodiments, the subject is at risk of developing cancer. In some embodiments, the subject has been treated for cancer. In some embodiments, the subject is being treated for cancer. In some embodiments, the subject is a cancer patient. In some embodiments, the subject is a cancer patient that is undergoing and/or being treated with chemotherapy.

In some embodiments, the subject is selected from the group consisting of a subject suspected of having cancer, a subject that has cancer, a subject diagnosed with cancer, a subject that is at risk of developing cancer, a subject that has been treated for cancer, and a subject that is being treated for cancer.

### Various Non-Limiting Embodiments of the Invention

Many people suffer from cancer and undergo treatment for cancer. As such there is a need for safer alternatives to many of the single-component radiation and chemotherapy approaches currently used for the treatment of cancer.

Boron neutron capture therapy (BNCT) is a promising binary therapeutic strategy for the treatment of cancer including but not limited to recalcitrant tumors such as brain tumors, pancreatic tumors, lung tumors, breast tumors, and liver tumors.

Without being bound by theory, the power of BNCT lies in the fact that both of its essential components, nonradioactive ¹⁰B nuclei and low-energy thermal neutrons, are nontoxic by themselves, however together they initiate an energetic nuclear decomposition reaction that typically cannot extend past the diameter of a cell. Based on this principle, BNCT is proposed as a safer alternative to many of the single-component radiation and chemotherapy approaches currently used for the treatment of cancer. However, several unresolved issues to the use of BNCT for the treatment of cancer still exist.

Recent advancements by TAE Technologies in the design of small-scale neutron generators promises to provide reliable and cost effective neutron sources that generate an optimal epithermal neutron beam of optimal energy for the treatment of deep-seated tumors using boron neutron capture therapy (BNCT). Moreover, these small-scale neutron generators will accelerate the implementation of boron neutron capture therapy (BNCT) in the clinical setting by providing reliable neutron sources to clinics.

A fundamental unresolved issue that limits the efficacy of boron neutron capture therapy (BNCT) to treat tumors is the poor tumor accumulation of current boron-containing compounds. This is of high importance as a poor accumulation of these compounds lowers the efficacy of boron neutron capture therapy (BNCT) to treat and kill tumors. Furthermore, the non-specific accumulation of the boron compounds in healthy tissue, even though it is not a problem by itself due to the non-toxicity of these boron compounds, can cause unexpected toxicity to healthy tissues upon exposure to the neutron beam. For these reasons, designing boron containing agents that can be targeted selectively and accumulate in high amount in tumors is of critical importance for the success and clinical translation of boron neutron capture therapy (BNCT).

In particular, there is an existing need for boron neutron capture therapy (BNCT) to selectively delivery large amounts of ¹⁰B nuclei to the tumor tissue (e.g., approximately 20-35 ug/g tumor or 109 atoms/cell). Existing boron neutron capture therapy (BNCT) agents are unable to meet this need.

Therefore, anon-limiting object of the present invention is to provide compositions, agents, articles of manufacture, nanovehicles, nanoparticles, methods, etc. for boron neutron capture therapy (BNCT) to treat cancer.

Another non-limiting object of the present invention is to provide nanoparticles comprising boron clusters.

Another non-limiting object of the present invention is to provide methods for treating cancer by boron neutron capture therapy (BNCT) using the compositions, agents, articles of manufacture, nanovehicles, nanoparticles of the present invention.

In various embodiments of the present invention, commercially available Feraheme samples were incubated with varied concentrations of B₁₂H₁₂ salts dissolved in water. A combination of NMR and ICP-AE spectroscopy was used to validate the loading quantities. Based on these studies we developed a protocol, which allows to load 10,0000-13,0000 boron clusters per each Feraheme nanoparticle. The boron loading obtained in our studies would result in the delivery of up to 760 ug of boron per gram of tissue. Importantly, this value represents more than a 20-fold increase in the boron delivery efficiency than the loading needed for BNCT to be a viable technology for cancer treatment. Furthermore, stability of these nanoparticles loaded with the boron clusters was evaluated at various conditions by ¹¹B NMR spectroscopy. No significant leaching was observed in neat water, PBS buffer (at pH 7.4 and pH 5.5) as well as 10% fetal bovine serum within a 24 hour time period.

### Nanovehicles, Compositions, and Articles of Manufacture

In various embodiments, the present invention provides a nanovehicle, comprising: a core, wherein the core comprises at least one iron oxide; a shell surrounding the core, wherein the shell comprises at least one polymer; and at least one boron cluster. In some embodiments, the nanovehicle is a nanoparticle.

In various embodiments, the present invention provides a composition, comprising: a core, wherein the core comprises at least one iron oxide; a shell surrounding the core, wherein the shell comprises at least one polymer; and at least one boron cluster. In some embodiments, the composition is a nanoparticle.

In various embodiments, present invention provides an article of manufacture, comprising: a core, wherein the core comprises at least one iron oxide; a shell surrounding the core, wherein the shell comprises at least one polymer; and at least one boron cluster. In some embodiments, the article of manufacture is a nanoparticle.

In various embodiments, the present invention provides a nanovehicle, comprising: at least one polymer; and at least one boron cluster. In some embodiments, the nanovehicle is a nanoparticle. In various embodiments, the present invention provides a composition comprising: at least one polymer; and at least one boron cluster. In various embodiments, the present invention provides an article of manufacture comprising: at least one polymer; and at least one boron cluster.

In various embodiments, the present invention provides a nanovehicle, comprising: at least one polymer; and at least one compound comprising boron. In various embodiments, the present invention provides a composition, comprising: at least one polymer; and at least one compound comprising boron. In various embodiments, the present invention provides an article of manufacture, comprising: at least one polymer; and at least one compound comprising boron.

In various embodiments, the present invention provides a nanovehicle, comprising: at least one polymer; and at least one boron cluster. In various embodiments, the present invention provides a composition, comprising: at least one polymer; and at least one boron cluster. In various embodiments, the present invention provides an article of manufacture, comprising: at least one polymer; and at least one boron cluster.

In some embodiments, the nanovehicle does not comprise iron oxide, cerium oxide, gold, or combinations thereof. In some embodiments, the composition does not comprise iron oxide, cerium oxide, gold, or combinations thereof. In some embodiments, the article of manufacture does not comprise iron oxide, cerium oxide, gold, or combinations thereof.

In some embodiments, the nanovehicle comprises at least one selected from iron oxide, cerium oxide, gold, and combinations thereof. In some embodiments, the composition comprises at least one selected from iron oxide, cerium oxide, gold, and combinations thereof. In some embodiments, the article of manufacture comprises at least one selected from iron oxide, cerium oxide, gold, and combinations thereof.

In various embodiments, the present invention provides a nanovehicle, comprising: ferumoxytol; and at least one boron cluster. In some embodiments, the ferumoxytol comprises carboxymethyl dextran. In various embodiments, the present invention provides a composition, comprising: ferumoxytol; and at least one boron cluster. In various embodiments, the present invention provides an article of manufacture, comprising: ferumoxytol; and at least one boron cluster.

In various embodiments, the present invention provides a nanovehicle, comprising: ferumoxytol; and at least one compound comprising boron. In various embodiments, the present invention provides a composition, comprising: ferumoxytol; and at least one compound comprising boron. In various embodiments, the present invention provides an article of manufacture, comprising: ferumoxytol; and at least one compound comprising boron.

In various embodiments, the present invention provides a nanovehicle, comprising: ferumoxytol; and at least one boron cluster. In various embodiments, the present invention provides a composition, comprising: ferumoxytol; and at least one boron cluster. In various embodiments, the present invention provides an article of manufacture, comprising: ferumoxytol; and at least one boron cluster.

In various embodiments, the present invention provides a nanovehicle, comprising at least one selected from the group consisting of Ferumoxytol, Ferumoxides, Ferucarbotran, Ferumoxtran-10, NC100150, VSOP C184, and combinations thereof; and at least one compound comprising boron. In various embodiments, the present invention provides a composition, comprising at least one selected from the group consisting of Ferumoxytol, Ferumoxides, Ferucarbotran, Ferumoxtran-10, NC100150, VSOP C184, and combinations thereof; and at least one compound comprising boron. In various embodiments, the present invention provides an article of manufacture, comprising at least one selected from the group consisting of Ferumoxytol, Ferumoxides, Ferucarbotran, Ferumoxtran-10, NC100150, VSOP C184, and combinations thereof; and at least one compound comprising boron.

In various embodiments, the present invention provides a nanovehicle, comprising at least one selected from the group consisting of Ferumoxytol, Ferumoxides, Ferucarbotran, Ferumoxtran-10, NC100150, VSOP C184, and combinations thereof; and at least one boron cluster. In various embodiments, the present invention provides a composition, comprising at least one selected from the group consisting of Ferumoxytol, Ferumoxides, Ferucarbotran, Ferumoxtran-10, NC100150, VSOP C184, and combinations thereof; and at least one boron cluster. In various embodiments, the present invention provides an article of manufacture, comprising at least one selected from the group consisting of Ferumoxytol, Ferumoxides, Ferucarbotran, Ferumoxtran-10, NC100150, VSOP C184, and combinations thereof; and at least one boron cluster

In various embodiments, the present invention provides a nanovehicle, comprising: a core; a coating surrounding the core; and at least one compound comprising boron. In various embodiments, the present invention provides a composition, comprising: a core; a coating surrounding the core; and at least one compound comprising boron. In various embodiments, the present invention provides an article of manufacture, comprising: a core; a coating surrounding the core; and at least one compound comprising boron.

In various embodiments, the present invention provides a nanovehicle, comprising: a core; a coating surrounding the core; and at least one boron cluster. In various embodiments, the present invention provides a composition, comprising: a core; a coating surrounding the core; and at least one boron cluster. In various embodiments, the present invention provides an article of manufacture, comprising: a core; a coating surrounding the core; and at least one boron cluster.

In various embodiments, the present invention provides a nanovehicle, comprising: a core, a shell surrounding the core; and at least one compound comprising boron. In various embodiments, the present invention provides a composition, comprising: a core, a shell surrounding the core; and at least one compound comprising boron. In various embodiments, the present invention provides an article of manufacture, comprising: a core, a shell surrounding the core; and at least one compound comprising boron.

In various embodiments, the present invention provides a nanovehicle, comprising: a core, a shell surrounding the core; and at least one boron cluster. In various embodiments, the present invention provides a composition, comprising: a core, a shell surrounding the core; and at least one boron cluster. In various embodiments, the present invention provides an article of manufacture, comprising: a core, a shell surrounding the core; and at least one boron cluster.

In some embodiments, the at least one compound comprising boron is at least one boron cluster.

In some embodiments, the coating comprises at least one selected from the group consisting of polymer, co-polymer, monomer, and combinations thereof.

In some embodiments, the shell comprises at least one selected from the group consisting of polymer, co-polymer, monomer, and combinations thereof.

In some embodiments, the core comprises at least one selected from the group consisting of iron oxide, cerium oxide, gold, and combinations thereof.

In some embodiments, the at least one polymer is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In some embodiments, the coating comprises at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In some embodiments, the shell comprises at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In various embodiments, the present invention provides a nanovehicle, comprising: a core, a coating surrounding the core; and at least one boron cluster. In various embodiments, the present invention provides a composition, comprising: a core, a coating surrounding the core; and at least one boron cluster. In various embodiments, the present invention provides an article of manufacture, comprising: a core, a coating surrounding the core; and at least one boron cluster. In some embodiments, the coating comprises at least one selected from the group consisting of polymer, co-polymer, monomer, and combinations thereof.

In various embodiments, the present invention provides a nanovehicle, comprising: at least one coated iron oxide particle; and at least one boron cluster. In various embodiments, the present invention provides a composition, comprising: at least one coated iron oxide particle; and at least one boron cluster. In various embodiments, the present invention provides an article of manufacture, comprising: at least one coated iron oxide particle; and at least one boron cluster.

In various embodiments, the present invention provides a nanovehicle, comprising: at least one coated iron oxide particle; and at least one compound comprising boron. In various embodiments, the present invention provides a composition, comprising: at least one coated iron oxide particle; and at least one compound comprising boron. In various embodiments, the present invention provides an article of manufacture, comprising: at least one coated iron oxide particle; and at least one compound comprising boron. In some embodiments, the at least one compound comprising boron is at least one boron cluster.

In various embodiments, the present invention provides a nanovehicle, comprising: at least one coated iron oxide particle; and at least one boron cluster. In various embodiments, the present invention provides a composition, comprising: at least one coated iron oxide particle; and at least one boron cluster. In various embodiments, the present invention provides an article of manufacture, comprising: at least one coated iron oxide particle; and at least one boron cluster.

In some embodiments, the at least one coated iron oxide particle is selected from the group consisting of Ferumoxytol, Ferumoxides, Ferucarbotran, Ferumoxtran-10, NC100150, VSOP C184, and combinations thereof.

In some embodiments, the nanovehicles, compositions, and/or articles of manufacture may optionally further comprise at least one targeting ligand.

In some embodiments, the nanovehicles, compositions, and/or articles of manufacture may optionally further comprise at least one therapeutic agent.

In some embodiments, the nanovehicles, compositions, and/or articles of manufacture selectively targets and/or binds to diseased tissue and/or diseased cells. In some embodiments, the nanovehicles, compositions, and/or articles of manufacture selectively targets and/or binds to cancerous tissue, cancer tissue, cancer cells, tumor, tumor tissue, tumor cells, and combinations thereof.

In some embodiments, the nanovehicles, compositions, and/or articles of manufacture selectively targets and/or binds to diseased tissue and/or diseased cells compared to non-diseased tissue and/or non-diseased cells. In some embodiments, the nanovehicles, compositions, and/or articles of manufacture selectively targets and/or binds to cancerous tissue, cancer tissue, cancer cells, tumor, tumor tissue, tumor cells, and combinations thereof compared to healthy tissue, non-cancererous tissue, non-cancerous cells.

In some embodiments, the nanovehicles, compositions, and/or articles of manufacture further comprise at least one targeting ligand. In some embodiments, the targeting ligand is attached to the shell.

In some embodiments, the nanovehicles, compositions, and/or articles of manufacture further comprise at least one drug.

In some embodiments, the nanovehicles, compositions, and/or articles of manufacture further comprise at least one fluorescent dye.

In some embodiments, the at least one boron cluster is encapsulated in the shell or in the coating. In some embodiments, the at least one boron cluster is encapsulated in the nanovehicle, nanoparticle, probe, composition, or article of manufacture. In some embodiments, the at least one boron cluster is attached to the shell or in the coating. In some embodiments, the at least one boron cluster is attached to the nanovehicle, nanoparticle, probe, composition, or article of manufacture.

In some embodiments, the nanovehicles, nanoparticles, probes, compositions, or articles of manufacture do not contain a targeting moiety. In some embodiments, the nanovehicles, nanoparticles, probes, compositions, or articles of manufacture do not comprise a targeting moiety.

### Iron Oxide Particles

Feraheme (FH), also known as Ferumoxytol, is an FDA-approved carboxymethyl dextran coated iron oxide nanoparticle formulation for the treatment of anemia. Feraheme (FH) is also used off-label as an MRI contrast agent. In various embodiments, Feraheme (FH) can be modified with targeting ligands (e.g., antibodies, peptides, or small molecule) to facilitate receptor mediated tumor accumulation or permeability through the brain blood barrier.

Non-limiting examples of coated iron oxide and/or coated iron oxide particles include Ferumoxytol (Feraheme®), Ferumoxides (Feridex® IV, Berlex Laboratories), Ferucarbotran (Resovist®, Bayer Healthcare), Ferumoxtran-10 (AMI-227 or Code-7227, Combidex®, AMAG Pharma; Sinerem®, Guerbet), NC100150 (Clariscan®, Nycomed) and (VSOP C184, Ferropharm).

In some embodiments, the at least one coated iron oxide and/or at least one coated iron oxide particle is selected from the group consisting of Ferumoxytol, Ferumoxides, Ferucarbotran, Ferumoxtran-10, NC100150, VSOP C184, and combinations thereof.

In some embodiments, the iron oxide is superparamagnetic iron oxide (SPIO).

Boron Clusters

In various embodiments, boron clusters of the present invention include but are not limited to any of the boron clusters, including any of the functionalized and/or unfunctionalized boron clusters, described and/or disclosed in the following publications the contents of which are incorporated herein by reference in their entirety: (a) Axtell, J. C.; Saleh, L. M. A.; Qian, E. A.; Wixtrom, A. I.; Spokoyny, A. M. “Synthesis and Applications of Perfunctionalized Boron Clusters”, Inorg. Chem. 2018, 57, 2333-2350; (b) Dziedzic, R. M.; Martin, J. L.; Axtell, J. C.; Saleh, L. M. A.; Yang, Y.; Messina, M.; Houk, K. N.; Spokoyny, A. M. “Cage-Walking: Vertex Differentiation by Palladium-Catalyzed Isomerization of B(9)-Bromo-meta-Carborane”, J Am. Chem. Soc. 2017, 139, 7729-7732; (c) Axtell, J. C.; Kirlikovali, K. O.; Jung, D.; Rheingold, A. L.; Spokoyny, A. M. “Metal-Free Peralkylation of the closo-Hexaborate Anion”, Organometallics, 2017, 36, 1204-1210; (d) Qian, E. Q.; Wixtrom, A. I.; Axtell, J. C.; Saebi, A.; Rehak, P.; Han, Y.; Moully, E. H.; Mosallaei, D.; Chow, S.; Messina, M.; Wang, J.-Y.; Royappa, A. T.; Rheingold, A. L.; Maynard, H. D.; Kral, P.; Spokoyny, A. M. “Atomically Precise Organomimetic Cluster Nanomolecules (OCNs) Assembled via Perfluoroaryl-Thiol SNAr Chemistry”, Nature Chem. 2017, 9, 333-340.

Synthetic Preparation. In various embodiments, boron clusters of the present invention as disclosed herein may be synthesized using any synthetic method available to one of skill in the art. Non-limiting methods for the preparation of boron clusters are described and/or disclosed in the following publications the contents of which are incorporated herein by reference in their entirety: (a) Axtell, J. C.; Saleh, L. M. A.; Qian, E. A.; Wixtrom, A. I.; Spokoyny, A. M. “Synthesis and Applications of Perfunctionalized Boron Clusters”, Inorg. Chem. 2018, 57, 2333-2350; (b) Dziedzic, R. M.; Martin, J. L.; Axtell, J. C.; Saleh, L. M. A.; Yang, Y.; Messina, M.; Houk, K. N.; Spokoyny, A. M. “Cage-Walking: Vertex Differentiation by Palladium-Catalyzed Isomerization of B(9)-Bromo-meta-Carborane”, J. Am. Chem. Soc. 2017, 139, 7729-7732; (c) Axtell, J. C.; Kirlikovali, K. O.; Jung, D.; Rheingold, A. L.; Spokoyny, A. M. “Metal-Free Peralkylation of the closo-Hexaborate Anion”, Organometallics, 2017, 36, 1204-1210; (d) Qian, E. Q.; Wixtrom, A. I.; Axtell, J. C.; Saebi, A.; Rehak, P.; Han, Y.; Moully, E. H.; Mosallaei, D.; Chow, S.; Messina, M.; Wang, J.-Y.; Royappa, A. T.; Rheingold, A. L.; Maynard, H. D.; Kral, P.; Spokoyny, A. M. “Atomically Precise Organomimetic Cluster Nanomolecules (OCNs) Assembled via Perfluoroaryl-Thiol SNAr Chemistry”, Nature Chem. 2017, 9, 333-340.

In some embodiments, the at least one boron cluster is selected from the group consisting of unfunctionalized boron cluster, functionalized boron cluster, and combinations thereof.

In some embodiments, the at least one boron cluster is selected from the group consisting of an unfunctionalized B₁₂ boron cluster, functionalized B₁₂ boron cluster, and combinations thereof.

In some embodiments, the unfunctionalized B₁₂ boron cluster is a compound having the formula: Mₚ B₁₂H₁₂, where, M is a cation; and p is an integer selected from 1 and 2.

In some embodiments, the at least one functionalized B₁₂ boron cluster is at least one perfunctionalized B₁₂ boron cluster.

In some embodiments, the at least one perfunctionalized B₁₂ boron cluster is selected from the group consisting of a homoperfunctionalized B₁₂ boron cluster, heteroperfunctionalized B₁₂ boron cluster, and combinations thereof.

In some embodiments, the at least one homoperfunctionalized B₁₂ boron cluster is a compound having the formula: Mₚ B₁₂X^{a}₁₂, where M is a cation; p is an integer selected from 1 and 2; and X^{a} is selected from CH₃, OH, F, Cl, Br, I; —OC(═O)NR¹, where R¹ is alkyl; —OC(═O)R², where R² is aryl; —OC(═O)R³, where R³ is selected from alkyl and aryl; —OR⁴, where R⁴ is selected from —CH₂-aryl and alkyl.

In some embodiments, the at least one homoperfunctionalized B₁₂ boron cluster is a compound having the formula: Mₚ B₁₂X^{a}₁₂, where M is a cation; p is an integer selected from 1 and 2; and X^{a} is selected from CH₃, OH, F, Cl, Br, I; —OC(═O)NR¹, where R¹ is alkyl; —OC(═O)R², where R² is Ph; —OC(═O)R³, where R³ is selected from CH₃ and Ph; —OR⁴, where R⁴ is selected from —CH₂Ph and alkyl.

In some embodiments, the at least one heteroperfunctionalized B₁₂ boron cluster is a compound having the formula: Mₚ B₁₂X^{b}₁₁Y^{a}₁, where M is a cation; p is an integer selected from 1 and 2; Y^{a}₁ is selected from H, OH, NO₂, NH₃, SH; —OR⁵, where R is alkyl; NR⁶₃, where R⁶ is alkyl; and X^{b} is selected from H, OH, and halide.

In some embodiments, the at least one heteroperfunctionalized B₁₂ boron cluster is a compound having the formula: Mₚ B₁₂X^{b}₁₁Y^{a}₁, where M is a cation; p is an integer selected from 1 and 2; Y^{a}₁ is selected from H, OH, NO₂, NH₃, SH; —OR⁵, where R is selected from n-C₃H₇, n-C₈H₁₇, n-C₁₂H₂₅; NR⁶₃, where R⁶ is selected from CH₃ and n-C₁₂H₂₅; and X^{b} is selected from H, OH, F, Cl, and Br.

In some embodiments, the cation M is selected from the group consisting of Li⁺, Na⁺, K⁺, Rb⁺, Cs⁺, [H₃O]⁺, [NH₄]⁺, [NⁿBu₄]⁺, [(PPh₃)₂N]⁺, [TEAH]⁺, fluorescent cationic dyes, and combinations thereof.

Non-limiting examples of fluorescent cationic dyes include acridine and its derivatives (e.g., 9-amino acridine), cationic cyanine dyes and their derivatives (e.g., IR780 and IR825), and/or rhodamine.

In various embodiments, the at least one boron cluster is selected from the group consisting of at least one B₄ boron cluster, at least one B₅ boron cluster, at least one B₆ boron cluster, at least one B₇ boron cluster, at least one B₅ boron cluster, at least one B₉ boron cluster, at least one B₁₀ boron cluster, at least one B boron cluster, at least one B₁₂ boron cluster, and combinations thereof.

In some embodiments the at least one B₄ boron cluster is a selected from at least one functionalized B₄ boron cluster, at least one unfunctionalized B₄ boron cluster, at least one substituted B₄ boron cluster, at least one unsubstituted B₄ boron cluster and combinations thereof.

In some embodiments the at least one B₅ boron cluster is a selected from at least one functionalized B₅ boron cluster, at least one unfunctionalized B₅ boron cluster, at least one substituted B₅ boron cluster, at least one unsubstituted B₅ boron cluster and combinations thereof.

In some embodiments the at least one B₆ boron cluster is a selected from at least one functionalized B₆ boron cluster, at least one unfunctionalized B₆ boron cluster, at least one substituted B₆ boron cluster, at least one unsubstituted B₆ boron cluster and combinations thereof.

In some embodiments the at least one B₇ boron cluster is a selected from at least one functionalized B₇ boron cluster, at least one unfunctionalized B₇ boron cluster, at least one substituted B₇ boron cluster, at least one unsubstituted B₇ boron cluster and combinations thereof.

In some embodiments the at least one B₈ boron cluster is a selected from at least one functionalized B₈ boron cluster, at least one unfunctionalized B₈ boron cluster, at least one substituted B₈ boron cluster, at least one unsubstituted B₈ boron cluster and combinations thereof.

In some embodiments the at least one B₉ boron cluster is a selected from at least one functionalized B₉ boron cluster, at least one unfunctionalized B₉ boron cluster, at least one substituted B₉ boron cluster, at least one unsubstituted B₉ boron cluster and combinations thereof.

In some embodiments the at least one B₁₀ boron cluster is a selected from at least one functionalized B₁₀ boron cluster, at least one unfunctionalized B₁₀ boron cluster, at least one substituted B₁₀ boron cluster, at least one unsubstituted B₁₀ boron cluster and combinations thereof.

In some embodiments the at least one B₁₁ boron cluster is a selected from at least one functionalized B₁₁ boron cluster, at least one unfunctionalized B₁₁ boron cluster, at least one substituted B₁₁ boron cluster, at least one unsubstituted B₁₁ boron cluster and combinations thereof.

In some embodiments the at least one B₁₂ boron cluster is a selected from at least one functionalized B₁₂ boron cluster, at least one unfunctionalized B₁₂ boron cluster, at least one substituted B₁₂ boron cluster, at least one unsubstituted B₁₂ boron cluster and combinations thereof.

In some embodiments, the at least one boron cluster comprises at least one selected from the group consisting of closo-[BₓHₓ]²⁻, hypocloso-[BₓHₓ]⁻, hypercloso-[BₓHₓ]⁰, and combinations thereof, wherein x is an integer selected from 6, 7, 8, 9, 10, 11, and 12.

In some embodiments, the at least one boron cluster comprises a cation Mₚ, wherein M is a cation, and p is an integer selected from 1 and 2.

In some embodiments, the at least one boron cluster is a neutral compound. In some embodiments, the at least one boron cluster is a salt.

In some embodiments, the at least one boron cluster is selected from the group consisting of a substituted boron cluster, unsubstituted boron cluster, unfunctionalized boron cluster, functionalized boron cluster, perfunctionalized boron cluster, homoperfunctionalized boron cluster, heteroperfunctionalized boron cluster, and combinations thereof.

In some embodiments, the at least one boron cluster is selected from at least one carborane, at least one functionalized carborane, at least one unfunctionalized carborane, at least one substituted carborane, at least one unsubstituted carborane, and combinations thereof.

In some embodiments, the at least one boron cluster is selected from at least one heteroborane, at least one functionalized heteroborane, at least one unfunctionalized heteroborane, at least one substituted heteroborane, at least one unsubstituted heteroborane, and combinations thereof.

In some embodiments, the at least one boron cluster is selected from at least one azaborane, at least one functionalized azaborane, at least one unfunctionalized azaborane, at least one substituted azaborane, at least one unsubstituted azaborane, and combinations thereof.

The terms “linked”, “joined”, “grafted”, “tethered”, “associated”, “attached”, “connected” and “conjugated” in the context of the nanovehicles of the invention or nanoparticles of the invention or the probes of the invention or compositions of the invention or articles of manufacture of the invention, are used interchangeably to refer to any method known in the art for functionally connecting boron clusters or a compound comprising boron to the nanovehicles, nanoparticles, probes, compositions, articles of manufacture or components thereof or the coated iron oxide nanoparticles or components thereof, including, without limitation, recombinant fusion, covalent bonding, non-covalent bonding, disulfide bonding, ionic bonding, hydrogen bonding, and electrostatic bonding.

Polymers

In some embodiments, the at least one polymer is at least one biocompatible polymer.

In some embodiments, the at least one polymer is at least one polysaccharide.

In some embodiments, the at least one polymer is one selected from the group consisting of at least one dextran, at least one unfunctionalized dextran, at least one functionalized dextran, at least one unsubstituted dextran, at least one substituted dextran, and combinations thereof.

In some embodiments, the at least one polymer is selected from the group consisting of carboxymethyl dextran, at least one dextran, and combinations thereof.

In some embodiments, the at least one polymer is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In some embodiments, the at least one polymer is poly(acrylic acid) (PAA).

Polysaccharides

In various embodiments, the at least one polymer is at least one polysaccharide.

In various embodiments, the at least one polysaccharide is selected from at least one dextran, at least one unfunctionalized dextran, at least one functionalized dextran, at least one unsubstituted dextran, at least one substituted dextran, and combinations thereof.

In some embodiments, the at least one polysaccharide is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

Dextrans

Dextrans are polysaccharides which have a linear backbone of α-linked d-glucopyranosyl repeating units. Three classes of dextrans can be differentiated by their structural features. The pyranose ring structure contains five carbon atoms and one oxygen atom. Class 1 dextrans contain the α(1→6)-linked d-glucopyranosyl backbone modified with small side chains of d-glucose branches with α(1→2), α(1→3), and α(1→4)-linkage. The class 1 dextrans vary in their molecular weight, spatial arrangement, type and degree of branching, and length of branch chains depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked d-glucopyranosyl units with α(1→3)-linked branches. Class 3 dextrans (mutans) have a backbone structure of consecutive α(1→3)-linked d-glucopyranosyl units with α(1→6)-linked branches.

In various embodiments, the at least one polymer is selected from the group consisting at least one dextran, at least one unfunctionalized dextran, at least one functionalized dextran, at least one unsubstituted dextran, at least one substituted dextran, and combinations thereof.

In various embodiments, the at least one polymer is selected from the group consisting of at least one dextran, carboxymethyl dextran, and combinations thereof.

In various embodiments, the at least one polymer is carboxymethyl dextran.

In some embodiments, the at least one dextran is selected from the group consisting of a class 1 dextran, a class 2 dextran, a class 3 dextran, and combinations thereof.

Targeting Ligand

The term “targeting ligand” includes without limitation any compound, moiety or residue having, or being capable to promote, a targeting activity (e.g., a selective binding) of the nanovehicles (e.g., nanoparticles) of the invention towards any biological or pathological site within a living body. Targets with which targeting ligands may be associated include tumors (e.g., cancerous tumors).

The targeting ligand may be synthetic, semi-synthetic, or naturally-occurring. Non-limiting examples of materials or substances which may serve as targeting ligands include antibodies, antibody fragments, aptamers, small molecules, receptor molecules, receptor binding molecules, glycoproteins and lectins; peptides, including oligopeptides and polypeptides; peptidomimetics; saccharides, including mono and polysaccharides; vitamins; steroids; steroid analogs; hormones; cofactors; bioactive agents and genetic material, including nucleosides, nucleotides and polynucleotides.

In various embodiments, the targeting ligand is at least one selected from the group consisting of an antibody, peptide, aptamer, small molecule, and a combination thereof.

In various embodiments, the targeting ligand can be bound to the nanoparticle, or a component of the nanoparticle through a covalent bond. In such cases, the specific reactive moiety that needs to be present on the nanoparticle or a component of the nanoparticle will depend on the particular targeting ligand to be coupled thereto.

In some embodiments, the targeting ligands selectively target and/or bind to diseased tissue and/or diseased cells. In some embodiments, the targeting ligands selectively target and/or bind to cancerous tissue, cancer tissue, cancer cells, tumor, tumor tissue, tumor cells, and combinations thereof.

In some embodiments, the targeting ligands selectively targets and/or binds to diseased tissue and/or diseased cells compared to non-diseased tissue and/or non-diseased cells. In some embodiments, the targeting ligands selectively targets and/or binds to cancerous tissue, cancer tissue, cancer cells, tumor, tumor tissue, tumor cells, and combinations thereof compared to healthy tissue, non-cancererous tissue, non-cancerous cells.

Treatment Methods

In various embodiments, the present invention provides a method of treating, preventing, reducing the likelihood of having, reducing the severity of and/or slowing the progression of a disease, disorder, or disease condition in a subject. The method may comprise or may consist of providing a nanovehicle (e.g., a nanoparticle) described herein and administering a therapeutically effective amount of the nanovehicle (e.g., nanoparticle) to the subject, thereby treating, preventing, reducing the likelihood of having, reducing the severity of and/or slowing the progression of the disease, disorder, or disease condition in the subject.

In various embodiments, the present invention provides a method of treating, reducing the severity of and/or slowing the progression of a disease, disorder, or disease condition in a subject, comprising: providing at least one nanovehicle, wherein the at least one nanovehicle comprises: a core, wherein the core comprises at least one iron oxide; a shell surrounding the core, wherein the shell comprises at least one polymer; and at least one boron cluster; administering a therapeutically effective amount of the at least one nanovehicle to the subject; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.

In various embodiments, the present invention provides a method of treating, reducing the severity of and/or slowing the progression of a disease, disorder, or disease condition in a subject, comprising: providing at least one nanovehicle, wherein the at least one nanovehicle comprises at least one polymer; and at least one boron cluster; administering a therapeutically effective amount of the at least one nanovehicle to the subject; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.

In various embodiments, the present invention provides a method of treating, reducing the severity of and/or slowing the progression of a disease, disorder, or disease condition in a subject, comprising: providing at least one nanovehicle, wherein the at least one nanovehicle comprises ferumoxytol; and at least one boron cluster; administering a therapeutically effective amount of the at least one nanovehicle to the subject; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.

In various embodiments, the present invention provides a method of treating, reducing the severity of and/or slowing the progression of a disease, disorder, or disease condition in a subject, comprising: providing at least one composition, at least one nanovehicle, or at least one article of manufacture of the present invention; administering a therapeutically effective amount of the at least one composition, at least one nanovehicle, or the at least one article of manufacture to the subject; and radiating the at least one composition, at least one nanovehicle, or the at least one article of manufacture with neutrons, thereby treating, reducing the severity of and/or slowing the progression of the disease, disorder, or disease condition in the subject.

In various embodiments, the present invention provides a method of treating, reducing the severity of and/or slowing the progression of a disease, disorder, or disease condition in a subject, comprising: providing at least one composition, at least one nanovehicle, or at least one article of manufacture of the present invention; administering a therapeutically effective amount of the at least one composition, at least one nanovehicle, or the at least one article of manufacture to the subject; and radiating the at least one composition, at least one nanovehicle, or the at least one article of manufacture with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.

In various embodiments, the present invention provides a method of treating, reducing the severity of and/or slowing the progression of a disease in a subject, comprising: providing at least one nanovehicle, wherein the at least one nanovehicle comprises: a core, wherein the core comprises at least one iron oxide; a shell surrounding the core, wherein the shell comprises at least one polymer; at least one boron cluster; and at least one targeting ligand attached to the shell; administering a therapeutically effective amount of the at least one nanovehicle to the subject, thereby contacting a tissue of the subject with the at least one nanovehicle, wherein the tissue is selected from the group consisting of non-diseased tissue and diseased tissue, and combinations thereof; wherein the nanovehicle selectively binds to the diseased tissue; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease in the subject

In various embodiments, the present invention provides a method of treating, reducing the severity of and/or slowing the progression of cancer in a subject, comprising: providing at least one nanovehicle, wherein the at least one nanovehicle comprises: a core, wherein the core comprises at least one iron oxide; a shell surrounding the core, wherein the shell comprises at least one polymer; at least one boron cluster; and at least one targeting ligand attached to the shell; administering a therapeutically effective amount of the at least one nanovehicle to the subject, thereby contacting a tissue of the subject with the at least one nanovehicle, wherein the tissue is selected from the group consisting of cancerous tissue, non-cancerous tissue, and combinations thereof; wherein the nanovehicle selectively binds to the cancerous tissue; and radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the cancer in the subject.

In various embodiments, the disease, disorder, or disease condition is a cancer. Examples of cancer include but are not limited to breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; cervical cancers such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; acute myeloid leukemia (AML), preferably acute promyleocytic leukemia in peripheral blood; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's sarcoma; Ewing sarcoma; central nervous system cancers such as primary brain tumor, which includes gliomas (astrocytoma, anaplastic astrocytoma, or glioblastoma multiforme (GBM)), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas; oral cavity and oropharyngeal cancer; stomach cancer such as lymphomas, gastric stromal tumors, and carcinoid tumors; testicular cancer such as germ cell tumors (GCTs), which include seminomas and nonseminomas; and gonadal stromal tumors, which include Leydig cell tumors and Sertoli cell tumors; head cancer; neck cancer; throat cancer; and thymus cancer, such as to thymomas, thymic carcinomas, Hodgkin disease, non-Hodgkin lymphomas carcinoids or carcinoid tumors. Also, the methods can be used to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer.

In some embodiments, the radiating is provided by a neutron generator.

In some embodiments, the low-energy thermal neutrons have a neutron energy of about 0.025 eV.

In some embodiments, the thermal neutrons have a neutron energy of about 0.025 eV.

In some embodiments, the epithermal neutrons have a neutron energy of about 0.025 eV to about 0.4 eV.

In some embodiments, the at least one nanovehicle is administered to deliver at least 20-35 ug ¹⁰B nuclei/g tumor to the subject. In some embodiments, the at least one composition is administered to deliver at least 20-35 ug ¹⁰B nuclei/g tumor to the subject. In some embodiments, the at least one article of manufacture is administered to deliver at least 20-35 ug ¹⁰B nuclei/g tumor to the subject.

In some embodiments, the at least one boron cluster is encapsulated in the at least one polymer or in the ferumoxytol. In some embodiments, the at least one boron cluster is linked to the at least one polymer or to the ferumoxytol. In some embodiments, the at least one boron cluster is linked to the at least one polymer or to the ferumoxytol by at least one linkage.

In some embodiments, the nanovehicle, composition, and/or article of manufacture further comprises at least one targeting ligand.

In some embodiments, the nanovehicle further comprises at least one targeting ligand. In some embodiments, the at least one targeting ligand is linked to the at least one polymer or to the ferumoxytol. In some embodiments, the at least one targeting ligand is linked to the at least one polymer or to the ferumoxytol by at least one linkage. In some embodiments, the targeting ligand is attached to the shell. In some embodiments, the targeting ligand is attached to the shell of the nanovehicle. In some embodiments, the targeting ligand is attached or linked to the shell of the nanovehicle by at least one linkage.

In some embodiments, the at least one nanovehicle is at least one nanoparticle.

Combination Therapies

In some embodiments, the methods may optionally further comprise simultaneously or sequentially providing additional therapies including by not limited to chemotherapy, radiation or a combination thereof.

Therapeutic Agents

In various embodiments, the nanovehicles (e.g., nanoparticles), compositions, and/or articles of manufacture provided herein may optionally further comprises a therapeutic agent loaded into the nanovehicles (e.g., nanoparticles), compositions, and/or articles of manufacture. In accordance with the present invention, non-limiting examples of the therapeutic agent include antineoplastic agents, blood products, biological response modifiers, anti-fungals, hormones, vitamins, peptides, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, radiosensitizers, immune checkpoint inhibitors, monoclonal antibodies, genetic material, antisense nucleic acids such as siRNA or RNAi molecules, drugs and prodrugs.

Pharmaceutical Compositions

The present invention also provides the nanovehicles (e.g., nanoparticles) described herein in the form of various pharmaceutical formulations. These pharmaceutical compositions may be used for treating, reducing the severity of and/or slowing the progression of a disease, disorder, or disease condition in a subject. In accordance with the invention, the disease, disorder, or disease condition can be a cancer.

In one embodiment, the present invention provides a pharmaceutical composition comprising at least one nanovehicle (e.g., at least one nanoparticle) described herein. In another embodiment, the present invention provides a pharmaceutical composition comprising at least two nanovehicles (e.g., nanoparticles) described herein. In still another embodiment, the present invention provides a pharmaceutical composition comprising a plurality of nanovehicles (e.g., nanoparticles) described herein. In accordance with the present invention, the nanovehicles (e.g., nanoparticle) comprises a targeting ligand conjugated thereto. In various embodiments, the pharmaceutical compositions also exhibit minimal toxicity when administered to a mammal.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In certain embodiments, the pharmaceutical composition is formulated for intravascular, intravenous, intraarterial, intratumoral, intramuscular, subcutaneous, intranasal, intraperitoneal, or oral administration.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

Kits

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least on nanovehicle (e.g., nanoparticle) described herein; and instructions for using the nanovehicles (e.g., nanoparticles) to detect, diagnose, treat, detect and treat, diagnose and treat, reduce the severity of and/or slow the progression of the disease, disorder, or disease condition in the subject. In some embodiments of the present invention, the nanovehicle (e.g., nanoparticle) comprises a targeting ligand conjugated thereto. In some embodiments of the present invention, the nanoparticle further comprises at least one drug. In some embodiments, the nanoparticle further comprises at least one fluorescent dye. In some embodiments, the nanoparticle further comprises at least one drug and at least one fluorescent dye.

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least one probe of the present invention described herein; and instructions for using the probes to detect, diagnose, treat, detect and treat, diagnose and treat, reduce the severity of and/or slow the progression of the disease, disorder, or disease condition in the subject. In some embodiments of the present invention, the probe further comprises at least one drug. In some embodiments, the probe further comprises at least one fluorescent dye. In some embodiments, the probe further comprises at least one drug and at least one fluorescent dye.

The kit is an assemblage of materials or components, including at least one of the inventive compositions and/or nanovehicles (e.g., nanoparticles) and/or nanoparticles and/or probes. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components

Some embodiments of the present invention can be defined as any of the following numbered paragraphs:

1. A nanovehicle, comprising:
a core, wherein the core comprises at least one iron oxide;
a shell surrounding the core, wherein the shell comprises at least one polymer; and
at least one boron cluster.
2. The nanovehicle of paragraph 1, wherein the at least one iron oxide is selected from the group consisting of FeO, Fe₂O₃, and a combination thereof.
3. The nanovehicle of paragraph 1, wherein the at least one polymer is at least one biocompatible polymer.
4. The nanovehicle of paragraph 1, wherein the at least one polymer is at least one polysaccharide.
5. The nanovehicle of paragraph 1, wherein the at least one polymer is one selected from the group consisting of at least one dextran, at least one unfunctionalized dextran, at least one functionalized dextran, at least one unsubstituted dextran, at least one substituted dextran, and combinations thereof.
6. The nanovehicle of paragraph 1, wherein the at least one polymer is selected from the group consisting of carboxymethyl dextran, at least one dextran, and combinations thereof.
7. The nanovehicle of paragraph 5 or paragraph 6, wherein the at least one dextran is selected from the group consisting of a class 1 dextran, a class 2 dextran, a class 3 dextran, and combinations thereof.
8. The nanovehicle of paragraph 1, wherein the at least one boron cluster is selected from the group consisting of unfunctionalized boron cluster, functionalized boron cluster, and combinations thereof.
9. The nanovehicle of paragraph 1, wherein the at least one boron cluster is selected from the group consisting of an unfunctionalized B₁₂ boron cluster, functionalized B₁₂ boron cluster, and combinations thereof.
10. The nanovehicle of paragraph 9, wherein the unfunctionalized B₁₂ boron cluster is a compound having the formula:

MₚB₁₂H₁₂,

where,
M is a cation; and
p is an integer selected from 1 and 2.
11. The nanovehicle of paragraph 9, wherein the at least one functionalized B₁₂ boron cluster is at least one perfunctionalized B₁₂ boron cluster.
12. The nanovehicle of paragraph 11, wherein the at least one perfunctionalized B₁₂ boron cluster is selected from the group consisting of a homoperfunctionalized B₁₂ boron cluster, heteroperfunctionalized B₁₂ boron cluster, and combinations thereof.
13. The nanovehicle of paragraph 12, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR¹, where R¹ is alkyl; —OC(═O)R², where R² is aryl; —OC(═O)R³, where R³ is selected from alkyl and aryl; —OR⁴, where R⁴ is selected from —CH₂-aryl and alkyl.
14. The nanovehicle of paragraph 12, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR¹, where R¹ is alkyl; —OC(═O)R², where R² is Ph; —OC(═O)R³, where R³ is selected from CH₃ and Ph; —OR⁴, where R⁴ is selected from —CH₂Ph and alkyl.
15. The nanovehicle of paragraph 12, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO₂, NH₃, SH; —OR⁵, where R⁵ is alkyl; NR⁶₃, where R⁶ is alkyl; and
X^{b} is selected from H, OH, and halide.
16. The nanovehicle of paragraph 12, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO₂, NH₃, SH; —OR⁵, where R is selected from n-C₃H₇, n-CH₁₇, n-C₁₂H₂₅; NR⁶₃, where R⁶ is selected from CH₃ and n-C₁₂H₂₅; and
X^{b} is selected from H, OH, F, Cl, and Br.
17. The nanovehicle of any one of paragraphs 1 to 16, wherein M is selected from the group consisting of Li⁺, Na⁺, K⁺, R_{b}⁺, Cs⁺, [H₃O]⁺, [NH₄]⁺, [NⁿBu₄]⁺, [(PPh₃)₂N]⁺, [TEAH]⁺, fluorescent cationic dyes, and combinations thereof.
18. The nanovehicle of paragraph 1, wherein the at least one boron cluster is selected from the group consisting of Na₂B₁₂H₁₁SH, Na₂B₁₂H₁₂, CsB₁₂H₁₁NH₃, Cs₂B₁₂H₁₂, [TEAH]₂B₁₂H₁₂, and combinations thereof.
19. The nanovehicle of paragraph 1, wherein the at least one boron cluster is encapsulated in the at least one polymer.
20. The nanovehicle of paragraph 1, wherein the at least one boron cluster is linked to the at least one polymer.
21. The nanovehicle of paragraph 1, wherein the at least one boron cluster is linked to the at least one polymer by at least one linkage.
22. The nanovehicle of paragraph 1, further comprising at least one targeting ligand.
23. The nanovehicle of paragraph 1, wherein the at least one targeting ligand is selected from the group consisting of an antibody, peptide, aptamer, small molecule and a combination thereof.
24. The nanovehicle of paragraph 22, wherein the at least one targeting ligand is linked to the at least one polymer.
25. The nanovehicle of paragraph 22, wherein the at least one targeting ligand is linked to the at least one polymer by at least one linkage.
26. The nanovehicle of any one of paragraphs 1 to 25, wherein the nanovehicle is a nanoparticle.
27. A nanovehicle, comprising:
at least one polymer; and
at least one boron cluster.
28. The nanovehicle of paragraph 27, wherein the at least one iron oxide is selected from the group consisting of FeO, Fe₂O₃, and a combination thereof.
29. The nanovehicle of paragraph 27, wherein the at least one polymer is at least one biocompatible polymer.
30. The nanovehicle of paragraph 27, wherein the at least one polymer is at least one polysaccharide.
31. The nanovehicle of paragraph 27, wherein the at least one polymer is one selected from the group consisting of at least one dextran, at least one unfunctionalized dextran, at least one functionalized dextran, at least one unsubstituted dextran, at least one substituted dextran, and combinations thereof.
32. The nanovehicle of paragraph 27, wherein the at least one polymer is selected from the group consisting of carboxymethyl dextran, at least one dextran, and combinations thereof.
33. The nanovehicle of paragraph 31 or paragraph 32, wherein the at least one dextran is selected from the group consisting of a class 1 dextran, a class 2 dextran, a class 3 dextran, and combinations thereof.
34. The nanovehicle of paragraph 27, wherein the at least one boron cluster is selected from the group consisting of unfunctionalized boron cluster, functionalized boron cluster, and combinations thereof.
35. The nanovehicle of paragraph 27, wherein the at least one boron cluster is selected from the group consisting of an unfunctionalized B₁₂ boron cluster, functionalized B₁₂ boron cluster, and combinations thereof.
36. The nanovehicle of paragraph 35, wherein the unfunctionalized B₁₂ boron cluster is a compound having the formula:

MₚB₁₂H₁₂,

where,
M is a cation; and
p is an integer selected from 1 and 2.
37. The nanovehicle of paragraph 35, wherein the at least one functionalized B₁₂ boron cluster is at least one perfunctionalized B₁₂ boron cluster.
38. The nanovehicle of paragraph 37, wherein the at least one perfunctionalized B₁₂ boron cluster is selected from the group consisting of a homoperfunctionalized B₁₂ boron cluster, heteroperfunctionalized B₁₂ boron cluster, and combinations thereof.
39. The nanovehicle of paragraph 38, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR¹, where R¹ is alkyl; —OC(═O)R², where R² is aryl; —OC(═O)R³, where R³ is selected from alkyl and aryl; —OR⁴, where R⁴ is selected from —CH₂-aryl and alkyl.
40. The nanovehicle of paragraph 38, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR¹, where R¹ is alkyl; —OC(═O)R², where R² is Ph; —OC(═O)R³, where R³ is selected from CH₃ and Ph; —OR⁴, where R⁴ is selected from —CH₂Ph and alkyl.
41. The nanovehicle of paragraph 38, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO2, NH₃, SH; —OR⁵, where R⁵ is alkyl; NR⁶₃, where R⁶ is alkyl; and
X^{b} is selected from H, OH, and halide.
42. The nanovehicle of paragraph 38, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO₂, NH₃, SH; —OR⁵, where R is selected from n-C₃H₇, n-CH₁₇, n-C₁₂H₂₅; NR⁶₃, where R⁶ is selected from CH₃ and n-C₁₂H₂₅; and
X^{b} is selected from H, OH, F, Cl, and Br.
43. The nanovehicle of any one of paragraphs 27 to 42, wherein M is selected from the group consisting of Li⁺, Na⁺, K⁺, R_{b}⁺, Cs⁺, [H₃O]⁺, [NH₄]⁺, [NⁿBu₄]⁺, [(PPh₃)₂N]⁺, [TEAH]⁺, fluorescent cationic dyes, and combinations thereof.
44. The nanovehicle of paragraph 27, wherein the at least one boron cluster is selected from the group consisting of Na₂B₁₂H₁₁SH, Na₂B₁₂H₂, CsB₁₂H₁₁NH₃, Cs₂B₁₂H₁₂, [TEAH]₂B₁₂H₂, and combinations thereof.
45. The nanovehicle of paragraph 27, wherein the at least one boron cluster is encapsulated in the at least one polymer.
46. The nanovehicle of paragraph 27, wherein the at least one boron cluster is linked to the at least one polymer.
47. The nanovehicle of paragraph 27, wherein the at least one boron cluster is linked to the at least one polymer by at least one linkage.
48. The nanovehicle of paragraph 27, further comprising at least one targeting ligand.
49. The nanovehicle of paragraph 27, wherein the at least one targeting ligand is selected from the group consisting of an antibody, peptide, aptamer, small molecule and a combination thereof.
50. The nanovehicle of paragraph 48, wherein the at least one targeting ligand is linked to the at least one polymer.
51. The nanovehicle of paragraph 48, wherein the at least one targeting ligand is linked to the at least one polymer by at least one linkage.
52. The nanovehicle of any one of paragraphs 27 to 51, wherein the nanovehicle is a nanoparticle.
53. A nanovehicle, comprising: ferumoxytol; and at least one boron cluster.
54. The nanovehicle of paragraph 53, wherein the ferumoxytol comprises carboxymethyl dextran.
55. The nanovehicle of paragraph 53, wherein the at least one boron cluster is selected from the group consisting of unfunctionalized boron cluster, functionalized boron cluster, and combinations thereof.
56. The nanovehicle of paragraph 53, wherein the at least one boron cluster is selected from the group consisting of an unfunctionalized B₁₂ boron cluster, functionalized B₁₂ boron cluster, and combinations thereof.
57. The nanovehicle of paragraph 56, wherein the unfunctionalized B₁₂ boron cluster is a compound having the formula:

MₚB₁₂H₁₂,

where,
M is a cation; and
p is an integer selected from 1 and 2.
58. The nanovehicle of paragraph 56, wherein the at least one functionalized B₁₂ boron cluster is at least one perfunctionalized B₁₂ boron cluster.
59. The nanovehicle of paragraph 58, wherein the at least one perfunctionalized B₁₂ boron cluster is selected from the group consisting of a homoperfunctionalized B₁₂ boron cluster, heteroperfunctionalized B₁₂ boron cluster, and combinations thereof.
60. The nanovehicle of paragraph 59, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR, where R¹ is alkyl; —OC(═O)R², where R² is aryl; —OC(═O)R³, where R³ is selected from alkyl and aryl; —OR⁴, where R⁴ is selected from —CH₂-aryl and alkyl.
61. The nanovehicle of paragraph 59, wherein the at least one homoperfunctionalized B₁₂ boron cluster is a compound having the formula:

MₚB₁₂X^{a}₁₂,

where M is a cation;
p is an integer selected from 1 and 2; and
X^{a} is selected from CH₃, OH, F, Cl, Br, I; —OC(═O)NR, where R¹ is alkyl; —OC(═O)R², where R² is Ph; —OC(═O)R³, where R³ is selected from CH₃ and Ph; —OR⁴, where R⁴ is selected from —CH₂Ph and alkyl.
62. The nanovehicle of paragraph 59, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO₂, NH₃, SH; —OR⁵, where R⁵ is alkyl; NR⁶₃, where R⁶ is alkyl; and
X^{b} is selected from H, OH, and halide.
63. The nanovehicle of paragraph 59, wherein the at least one heteroperfunctionalized B₁₂ boron cluster is a compound having the formula:

MₚB₁₂X^{b}₁₁Y^{a}₁,

where M is a cation;
p is an integer selected from 1 and 2;
Y^{a}₁ is selected from H, OH, NO₂, NH₃, SH; —OR⁵, where R is selected from n-C₈H₇, n-CH₁₇, n-C₁₂H₂₅; NR⁶₃, where R⁶ is selected from CH₃ and n-C₁₂H₂₅; and
X^{b} is selected from H, OH, F, Cl, and Br.
64. The nanovehicle of any one of paragraphs 53 to 63, wherein M is selected from the group consisting of Li⁺, Na⁺, K⁺, Rb⁺, Cs⁺, [H₃]⁺, [NH₄]⁺, [NⁿBu₄]⁺, [(PPh₃)₂N]⁺, [TEAH]⁺, fluorescent cationic dyes, and combinations thereof.
65. The nanovehicle of paragraph 53, wherein the at least one boron cluster is selected from the group consisting of Na₂B₁₂H₁₁SH, Na₂B₁₂H₁₂, CsB₁₂H₁₁NH₃, Cs₂B₁₂H₁₂, [TEAH]₂B₁₂H₂, and combinations thereof.
66. The nanovehicle of paragraph 54, wherein the at least one boron cluster is encapsulated in the carboxymethyl dextran.
67. The nanovehicle of paragraph 54, wherein the at least one boron cluster is linked to the carboxymethyl dextran.
68. The nanovehicle of paragraph 54, wherein the at least one boron cluster is linked to the carboxymethyl dextran by at least one linkage.
69. The nanovehicle of paragraph 53, further comprising at least one targeting ligand.
70. The nanovehicle of paragraph 69, wherein the at least one targeting ligand is selected from the group consisting of an antibody, aptamer, peptide, small molecule and a combination thereof.
71. The nanovehicle of paragraph 54, wherein the at least one targeting ligand is linked to the carboxymethyl dextran.
72. The nanovehicle of paragraph 54, wherein the at least one targeting ligand is linked to the carboxymethyl dextran by at least one linkage.
73. The nanovehicle of any one of paragraphs 53 to 72, wherein the nanovehicle is a nanoparticle.
74. A pharmaceutical composition comprising at least one nanovehicle of any one of paragraphs 1 to 73.
75. The pharmaceutical composition of paragraph 74, further comprising at least one pharmaceutically acceptable excipient.
76. The pharmaceutical composition of paragraph 74, further comprising at least one pharmaceutically acceptable carrier.
77. A method of treating, reducing the severity of and/or slowing the progression of a disease, disorder, or disease condition in a subject, comprising:
providing at least one nanovehicle, wherein the at least one nanovehicle comprises: a core, wherein the core comprises at least one iron oxide; a shell surrounding the core, wherein the shell comprises at least one polymer; and at least one boron cluster;
administering a therapeutically effective amount of the at least one nanovehicle to the subject; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.
78. A method of treating, reducing the severity of and/or slowing the progression of a disease, disorder, or disease condition in a subject, comprising:
providing at least one nanovehicle, wherein the at least one nanovehicle comprises at least one polymer; and at least one boron cluster;
administering a therapeutically effective amount of the at least one nanovehicle to the subject; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.
79. A method of treating, reducing the severity of and/or slowing the progression of a disease, disorder, or disease condition in a subject, comprising:
providing at least one nanovehicle, wherein the at least one nanovehicle comprises ferumoxytol; and at least one boron cluster;
administering a therapeutically effective amount of the at least one nanovehicle to the subject; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.
80. The method of any one of paragraphs 77 to 79, wherein the disease, disorder, or disease condition is cancer.
81. The method of anyone of paragraphs 77 to 79, wherein the radiating is provided by a neutron generator.
82. The method of any one of claims 77 to 79, wherein the low-energy thermal neutrons have a neutron energy of about 0.025 eV.
83. The method of any one of paragraphs 77 to 79, wherein the thermal neutrons have a neutron energy of about 0.025 eV.
84. The method of any one of paragraphs 77 to 79, wherein the epithermal neutrons have a neutron energy of about 0.025 eV to about 0.4 eV.
85. The method of any one of paragraphs 77 to 79, wherein the at least one nanovehicle is administered to deliver at least 20-35 ug ¹⁰B nuclei/g tumor to the subject.
86. The method of any one of paragraphs 77 to 79, wherein the at least one boron cluster is encapsulated in the at least one polymer or in the ferumoxytol.
87. The method of any one of paragraphs 77 to 79, wherein the at least one boron cluster is linked to the at least one polymer or to the ferumoxytol.
88. The method of any one of paragraphs 77 to 79, wherein the at least one boron cluster is linked to the at least one polymer or to the ferumoxytol by at least one linkage.
89. The method of any one of paragraphs 77 to 79, the nanovehicle further comprises at least one targeting ligand.
90. The method of any one of paragraphs 77 to 79, wherein the at least one targeting ligand is selected from the group consisting of an antibody, peptide, aptamer, small molecule and a combination thereof.
91. The method of any one of paragraphs 77 to 79, wherein the at least one targeting ligand is linked to the at least one polymer or to the ferumoxytol.
92. The method of any one of paragraphs 77 to 79, wherein the at least one targeting ligand is linked to the at least one polymer or to the ferumoxytol by at least one linkage.
93. The method of any one of paragraphs 77 to 92, wherein the at least one nanovehicle is at least one nanoparticle.
94. The method of any one of paragraphs 77 to 93, wherein the method is boron neutron capture therapy

### Various Non-Limiting Embodiments of the Invention

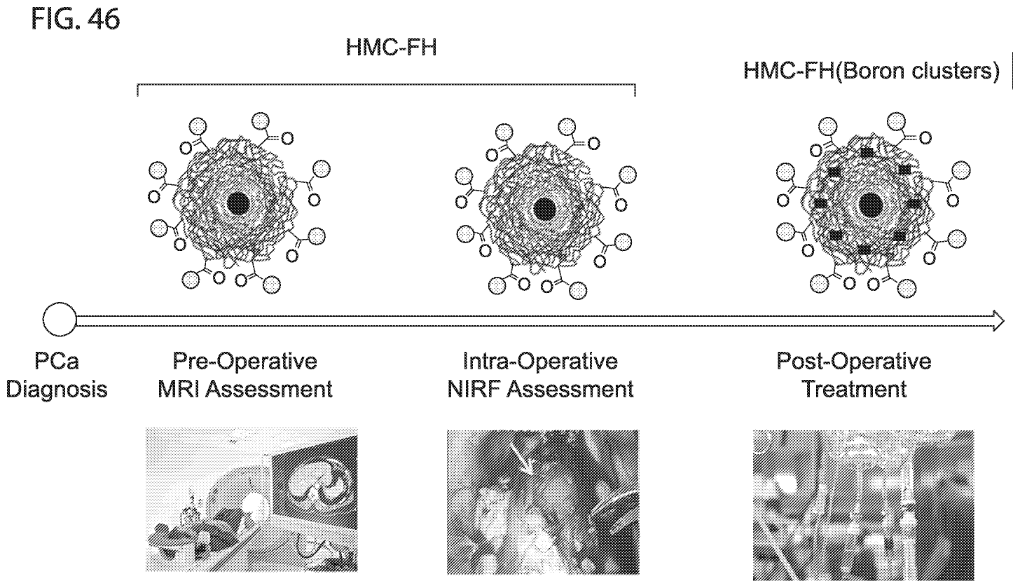

In various embodiments, the present invention relates to the development of an iron oxide nanoparticle based platform technology that would allow for (1) an MRI-based pre-surgery assessment of a tumor location and margins, (2) a fluorescent image-guided visualization of the tumor during surgery, and (3) and effective post-surgery chemotherapy regime to treat remaining primary tumor as well as metastatic lesions (FIG. 46). MRI is among the best pre-operative imaging technologies for PCa due to its high spatial and contrast resolution and the lack of ionizing radiation.^{[1]} It is typically used to determine the extent of the disease via the acquisition of a combination of T2-weighted and diffusion-weighted images. In addition, dynamic contrast-enhanced MRI using iron oxide nanoparticle formulations such as Feraheme (FH) would results in enhancement in tumor contrast and better detection on tumor margins and degree of tumor vascularization. Meanwhile, fluorescence imaging is the most promising approach for the intraoperative resection of tumors and sentinel lymph node metastasis.^{[2-12]} Intraoperative fluorescence-imaging provide guidance during cancer surgery for the complete resection of tumors with high sensitivity by identifying tumor margins during surgery. It is imperative that most if not all of the cancer tissue is taken out. For this to be accomplished, highly fluorescent agents that localize specifically to cancer are needed. However, even after successful resection of cancerous tissues, there is always the possibility that tissue, not-identified as cancerous during surgery, remains or cancer cells have already migrated through the lymphatic system to other organs to establish metastasis. Therefore, post-surgical chemotherapy typically is administered, resulting in an improved outcome and survival, minimizing recurrences and the establishment of metastasis.^{[13]} It would be highly advantageous, to utilize a nanoparticle based system that can aid in the visualization of tumors both pre-surgery and during surgery, while using the same nanoparticle platform technology as delivery system to deliver boron clusters and an optional drug post-operatively. In various embodiments of the present invention, we disclose the use of a Feraheme (FH) based image-guided system for both the pre-operative and intra-operative assessment of PCa tumor margins as well as the post-surgical treatment and assessment of boron cluster and an optional drug delivery to primary and secondary (metastasis) tumors using the same FH-based agent. To prove the use of our technology in cancer, we have done initial studies using cell cultures and mouse models of prostate and brain (glioblastoma) tumors. However, the technology can be used for the imaging and treatment of other solid tumors such as those from lungs, breast, ovaries, pancreas, head and neck, and skin among others.

In various embodiments, the present invention is based on the use of Feraheme (FH), an FDA-approved iron oxide nanoparticle formulation Feraheme (FH), also known as Ferumoxytol, is currently used in the clinic to treat iron deficiency anemia.^{[14]} FH is typically administered in two doses of 510 mg of iron each, between 3-8 days, for a total dose of 1020 mg Fe per treatment. The pharmacokinetics, biodistribution and safety profile of FH has been extensively studied, showing minimal toxicity in animal and humans subjects, being metabolized as regular iron by the liver within 6-8 weeks.^{[}15, 16] In addition, FH is increasingly used off-labeled in MR angiography and liver imaging due to its superparamagnetic properties, at doses far below those used for anemia treatment.^{[}17-19] Toxicity studies have shown that even a 12-fold higher than the clinical dose of FH present no significant toxicity with very few side effects being reported in adult cases.^{[16, 20]} Among those, anaphylaxis and hypersensitivity reactions are the most serious ones, but these problems have been minimized by administering FH as a diluted IV infusion over a period of 15 minutes or more as opposed to an undiluted bolus administration as it was administered in the past. In general, the use of FH is safe.^{[16]} In addition, iron oxide nanoparticles have been widely studied as magnetic sensors and most recently as drug delivery agents. Polymer coated iron oxide nanoparticles can encapsulate a hydrophobic cargo such as drugs (Taxol, Doxorubicin) or fluorescence dyes (DiI, DiR) within the nanoparticle's polymer coating (dextran or polyacrylic acid).^{[21]} The stable encapsulation of these cargos occurs at physiological pH within hydrophobic pockets in the nanoparticle's polymeric coating via hydrophobic and electrostatic interactions. At pH 6.5 or below, release of the cargo occurs, either fluorescently labeling the cell or causing cell death, when either a fluorescent dye or a cytotoxic drug was encapsulated respectively. Feraheme (FH) itself can be used as a drug delivery vehicle and that its superparamagnetic properties allow for MR-guided assessment of nanoparticle accumulation and drug release.^{[22]} In addition, our data shows that a FH-encapsulated drug is more efficient in reducing the size of tumors than the drug alone. These results were similar with all encapsulated drug such as doxorubicin, paclitaxel and bortezomib.

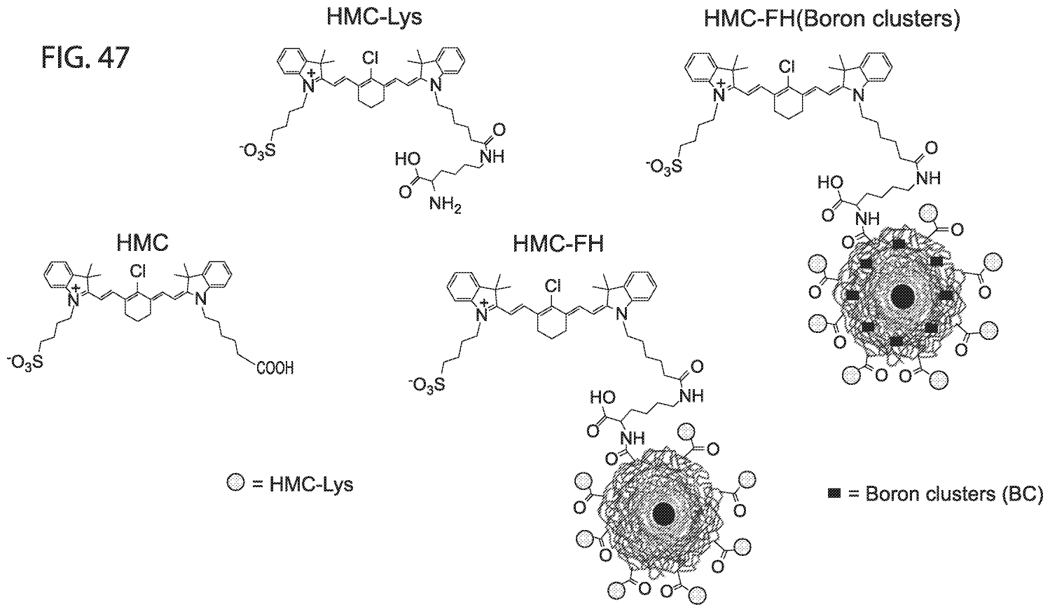

Even though tumor accumulation of nanoparticle via Enhanced Permeability and Retention (EPR) effect is widely recognized to be effective for nanoparticle-boron cluster delivery, it is not universal for all tumors. Furthermore, crossing the brain blood/tumor barrier is a challenge to overcome when treating brain tumors such as glioblastomas. Tumor targeting and enhanced brain blood barrier transcytosis can occur via receptor mediated targeting, which is facilitated by the attachment of targeting ligands to the nanoparticle surface.^{[23, 24]} In FH, carboxylic acid groups on the nanoparticle surface can be further modified with targeting ligands for specific targeting and accumulation in tumors. Of a wide selection of ligands that one can choose to target tumors, we selected the heptamethine carbocyanine (HMC) ligand to conjugate to FH (FIG. 47). HMC targets the organic anion transporter peptides (OATPs) which are a superfamily of transmembrane glycoproteins overexpressed in various tumors.^{[25, 26]} The OATPs family of proteins is composed of various subtypes including 11 known human OATPs classified into 6 subfamilies based on their amino acid sequence homologies.^{[25, 27]} For example, the OATPIB3 and OATPIA2 subtypes have been shown to be overexpressed in prostate cancer,^{[28, 29]} while OATPIA2 and OATP2B1 have been found to be expressed in brain tumors and brain metastasis.^{[25, 27, 28]} OATPs facilitate the transport of several substances into cells, including drugs and hormones.^{[25, 27]} Although the actual mechanism of HMC uptake by multiple tumors has not been fully elucidated, it is believed that the selective overexpression of multiple subtypes of OATPs in tumors contribute to the HMC ligand uptake by tumors. For example, it has been demonstrated that the overexpression of OATPIB3 mediate the selective uptake of HMC ligands in prostate cancer cells, but not in normal prostate epithelial cells.^{[30]} Therefore, the OATPIB3 subtype may be the transporter predominantly involved in the selective uptake of HMC in prostate cancer. HMC is a unique ligand because it also exhibits near infrared fluorescence (NIRF), with excitation in 750 nm and emission in 800. The dual NIRF imaging and OATP-targeting capability of HMC is unique and upon conjugation to Feraheme will endow FH with dual NIRF- and MR-imaging capabilities, as well as OATP-targeting ability. In addition to HMC unique NIRF properties, it has been shown that this ligand preferentially accumulates in a variety of cancer cells, but not normal cells as demonstrated in a variety of cancer cell lines, tumor xenografts, spontaneous mouse tumors in transgenic animals and human tumor samples.^{[31-33]} The HMC uptake has also been found to be mediated by tumor hypoxia and activated (HIF1α)/OATP signaling.^{[34]} Given that hypoxia and aberrant expression of OATPs is shared by multiple types of tumors and their metastatic lesion, conjugation of HMC on the surface on Feraheme will facilitate the pre-operative detection of tumors by MRI and the intraoperative detection of tumor margins by fluorescence imaging, while allowing for the post-surgical delivery of boron clusters and an optional drug to primary and secondary (metastasis) tumors.

Prostate Cancer (PCa). Challenges in PCa treatment. PCa remains one of the leading causes of death in men in the USA and around the world.^{[35, 36]} Current cancer chemotherapeutics, along with antiandrogen therapy, have improved the long-term survival of these patients.^{[13]} However, surgical removal of the cancerous tissue continues to be the most effective approach, resulting in curative results when complete removal of the cancerous tissue is achieved and no metastasis to nearby lymph nodes and other organs have occurred. Currently, complete removal of the cancer tissue in prostate cancer is challenging due to the location and proximity of the prostate gland to other organs such as the bladder, rectum, urethra and prostatic nerves. These issues limit the adaptation of wide surgical margins during prostatectomy, often resulting in positive surgical margins in up to 48% of the cases that require the use of post-surgery adjuvant chemotherapy using Docetaxel (DXT) and prednisone.^{[37, 38]}

Glioblastoma Multiforme (GBM). Challenges in glioblastoma treatment. Despite advances in surgical resection, chemotherapy and radiation treatment of glioblastoma multiforme (GBM), the overall median survival is estimated to be only about 15 months with a five-year survival rate of 10% after radiation therapy and chemotherapy^{[39-41]}. GBM can affect both men and women equally and at any age. This statistic makes GBM one of the most lethal and aggressive cancers. Standard of care starts with surgery, to eliminate most of the tumor mass, followed by a combination of chemo and radiation therapy to eradicate any residual tumor tissue. Alkylating agents such as temozolomide, in combination with surgical tumor resection and radiotherapy have increased the overall survival of newly diagnosed patients, but only by expanding survival by a couple of months. Unfortunately, tumor recurrence often develops within a few months after treatment due to difficulties in establishing tumor margins during surgery and in inefficient post-surgical treatments using chemotherapy. The failure of most chemotherapies to treat GBM is due to the ineffective ability of most drugs to cross the brain blood barrier (BBB) within the tumor area, more specifically the brain tumor area. Most problematic, recurrent tumors after failed chemotherapy are typically resistant to both classical chemotherapy and radiation therapy ^{[42-45]}, which makes treatment even more difficult. For these reasons, developing a nanoparticle based therapeutics that can (1) facilitate the visualization of tumors by MRI and fluorescent imaging pre and during surgery respectively, while (2) delivering potent chemotherapeutic drugs to the brain tumor are desperately needed. Overall, taxanes such as docetaxel and paclitaxel have been beneficial in the treatment most tumors, except for brain tumors due to the inability of these drugs to cross the brain blood barrier. Even though a taxane nanoformulation (Abraxane®) to treat other tumors via the EPR has been used to successfully treat other tumors, this formulation does not cross the BBB and it is not effective in treating GBM. For this reason, a nanoformulation that can deliver a taxane (DXT, PXL) to GBM cells by crossing the BBB would be a most needed improvement in the treatment of GBM. In this invention we report the use of HMC-FH(Boron Cluster+Drug) to deliver taxanes to GBM. Other drugs and compounds that typically do not cross the BBB such as Cabozentanib, Brefeldin A, Bortexomib and boron clusters, among others could be delivered to brain tumors using the same platform technology.

In some embodiments, the drug is a boron cluster. In some embodiments, the drug is a compound comprising boron. In some embodiments, the drug comprises a boron cluster. In some embodiments, the drug comprises a compound comprising boron. In some embodiments, the drug contains a boron cluster. In some embodiments, the drug contains a compound comprising boron.

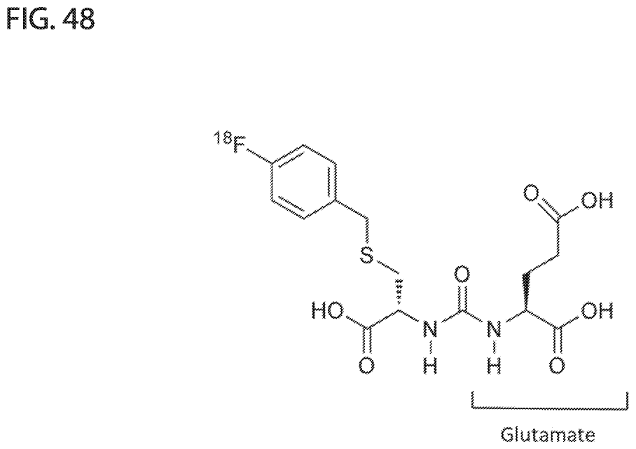

In various embodiments, the present invention relates to the use of conjugates of iron oxide nanoparticles with folic acid or glutamic acid for the multimodal detection of prostate cancer via direct targeting of the prostate specific membrane antigen (PSMA), which is overexpressed in both primary and metastatic prostate cancer as well as the neovasculature of most solid tumors, including breast, and lung, among others. PSMA has gained increasing interest as a molecular target for imaging as well as for the delivery of targeted cancer therapeutics. PSMA is a cell surface protein known to have a dual enzymatic activity of folate hydrolysate and glutamate carboxylase. PSMA binds folic acid, glutamic acid, and polyglutamated folates and facilitates the internalization of these molecules into cancer cells. Glutamic acid (glutamate) based molecule have been more extensively used to target PSMA than folic acid (folate) molecules. Indeed, various glutamate urea based probes have been designed to deliver optical and PET imaging agent (18F and 68Ga) to PCa tumors via PSMA. FIG. 48 shows the structure of one of these PSMA targeting imaging agents, 18F-DCFBC, where the glutamate moiety facilitates binding to PSMA.

In this invention, glutamate (or folate) is covalently bound to iron oxide nanoparticle (Feraheme) to image PSMA in prostate cancer tumors. A commercial and FDA-approved formulation of carboxymethyl dextran iron oxide nanoparticles, Feraheme (Ferumoxytol), was used in our invention. It is understood, however, that other versions of iron oxide nanoparticles can be also used besides Feraheme. Feraheme is used in the clinic to treat iron deficiency (anemia), but it is increasingly being used in MR-angiography and liver imaging.

In various embodiments of the present invention, the carboxylic acid groups on the surface of the Feraheme nanoparticles were conjugated to the amino group in glutamate to yield the Glu-Feraheme (GLU-FH) NP using EDC/NHS chemistry. Meanwhile, as folate does not have a functional amino group to conjugate directly to Feraheme, Folate-PEG-amine is used instead to yield Folate-PEG-Feraheme.

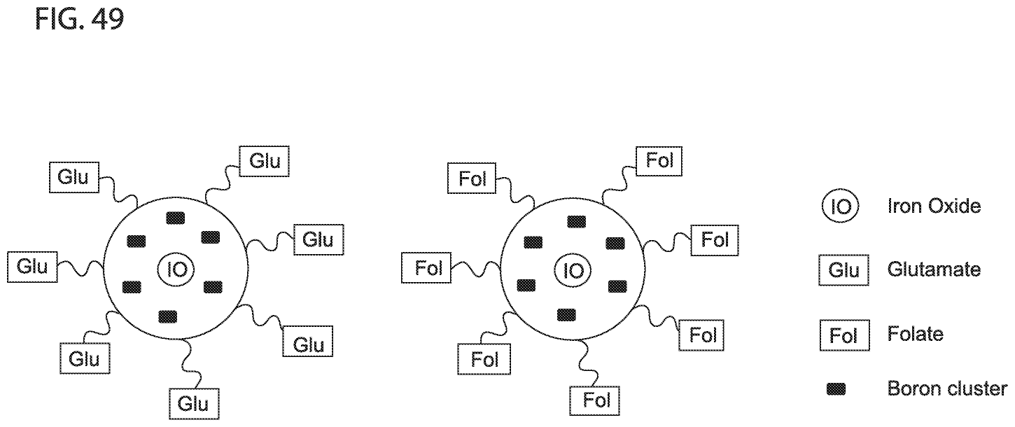

In another embodiment of the present invention, a theranostic nanoparticle has been developed (FIG. 49) by encapsulating a boron cluster within the carboxymethyl dextran coating of the PSMA targeting-Feraheme NPs. Folate ligands were attached to target the folate receptor. In various embodiments of the present invention, in addition to folic acid, glutamic acid is used to target the Feraheme nanoparticles to prostate cancer via PSMA. Therefore, Glutamate-Feraheme and Folate-Feraheme (Fol-FH) were synthesized and tested to target prostate cancer via PSMA for imaging and/or as a therapeutic to deliver boron clusters to prostate cancer. In addition, polyacrylic acid coated iron oxide nanoparticle can encapsulate or entrap boron clusters within the polymeric coating, creating a multimodal and theranostic nanoparticle.

In various embodiments, the present invention relates to the use of conjugates of iron oxide nanoparticles with at least one Angiopep. An Angiopep is a peptide that has been described in the literature to cross the brain blood barrier (BBB). Non-limiting examples of Angiopeps include Angiopep-1, Angiopep-2, Angiopep-5, or Angiopep-7. In some embodiments, at least one Angiopep is selected from Angiopep-1, Angiopep-2, Angiopep-5, Angiopep-7, and combinations thereof. Angiopep-2 is a 19 amino acid peptide (TFFYGGSRGKRNNFKTEEY) (SEQ ID NO: 2) that binds to the low-density lipoprotein receptor-related protein 1 (LRP-1), which is highly expressed in the brain endothelial cells of the BBB. Upon binding of Angiopep to LRP-1, the whole complex crosses the BBB via a transcytosis mechanism. Transcytosis typically enables the transport of proteins through the BBB via the formation of membrane-bound vesicles. In the case of LRP-1, these vesicles form upon binding of lipoproteins to this receptor on the apical side of the endothelia and quickly move to the basolateral side where the vesicles fuse with the membrane, releasing the cargo within the brain. Furthermore, glioblastoma multiforme (GBM) and other forms of malignant brain tumors have been found to have increased expression of LRP-1. In the particular case of GBM, studies have found that LRP-1 induces the expression of matrix metalloproteinase 2 (MMP2) and MMP9, promoting migration and invasion of human GBM cells (U87). Therefore, LRP-1 is an excellent target to facilitate the crossing of nanotherapeutics through the BBB, as well as their binding and internalization within brain cancer cells. Angiopep has been found to bind to LRP-1 and transcytose across the BBB.

Angiopep-1 is a peptide with the following amino acid sequence: TFFYGGCRGKRNNFKTEEY (SEQ ID NO: 3).

Angiopep-2 is a peptide with the following amino acid sequence: TFFYGGSRGKRNNFKTEEY (SEQ ID NO: 2).

Angiopep-5 is a peptide with the following amino acid sequence: TFFYGGSRGKRNNFRTEEY (SEQ ID NO: 4).

Angiopep-7 is a peptide with the following amino acid sequence: TFFYGGSRGRRNNFRTEEY (SEQ ID NO: 5).

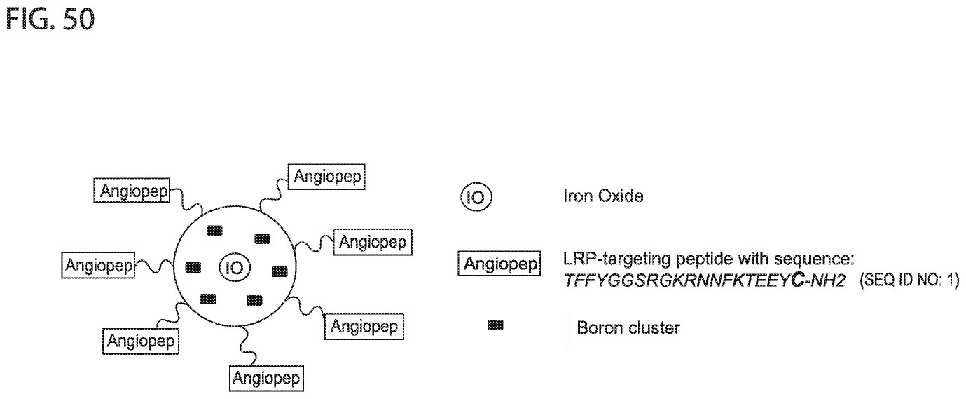

In various embodiments of the present invention, Feraheme (Ferumoxytol) a commercial and FDA-approved formulation of carboxymethy dextran iron oxide nanoparticles, was conjugated with Angiopep-2 and encapsulated with a boron cluster for the delivery of this cargo through the BBB (FIG. 50). By conjugating Angiopep-2 to Feraheme, an Angiopep-Feraheme nanoparticle conjugate will be produced with the following properties: 1. LRP-1 mediated transcytosis of Feraheme across the BBB; and 2. The use of Angiopep-Feraheme to deliver a cargo across the BBB.

In various embodiments of the present invention, we have conjugated Angiopep to the surface of Feraheme. Angiopep is a peptide that target the LRP-1 receptors which is overexpressed on the brain blood barrier (BBB) and on the cells of most brain tumors. The resulting Angiopep-Feraheme nanoparticle can then encapsulate boron clusters among other cargos (e.g., drugs, fluorescent dyes, etc.), for their delivery across the BBB and into brain tumor cells. In various embodiments of the present invention, nanoparticle delivery or boron cluster delivery to the brain can be monitored by MRI, as the magnetic properties of Feraheme allows for the monitoring of nanoparticle localization via MRI. In some embodiments, Angiopep is selected from the group consisting of Angiopep-1, Angiopep-2, Angiopep-5, and Angiopep-7, and combinations thereof. In some embodiments, Angiopep is Angiopep-2.

Nanoparticles, Compositions, and Articles of Manufacture

In various embodiments, the present invention provides a nanoparticle, comprising: a core, wherein the core comprises at least one iron oxide; a shell surrounding the core, wherein the shell comprises at least one polymer; at least one boron cluster; and at least one targeting moiety attached to the shell. In some embodiments, at least one boron cluster is encapsulated in the at least one polymer. In some embodiments, at least one boron cluster is linked to the at least one polymer.

In various embodiments, the present invention provides a nanoparticle, comprising: a core, wherein the core comprises at least one iron oxide; a shell surrounding the core, wherein the shell comprises at least one polymer; and at least one boron cluster. In some embodiments, the nanoparticle further comprises at least one targeting moiety. In some embodiments, the targeting moiety is attached to the shell.

In various embodiments, the present invention provides a composition, comprising: a core, wherein the core comprises at least one iron oxide; a shell surrounding the core, wherein the shell comprises at least one polymer; at least one boron cluster; and at least one targeting moiety attached to the shell. In some embodiments, the composition is a nanoparticle. In some embodiments, at least one boron cluster is encapsulated in the at least one polymer. In some embodiments, at least one boron cluster is linked to the at least one polymer.

In various embodiments, the present invention provides a composition, comprising: a core, wherein the core comprises at least one iron oxide; a shell surrounding the core, wherein the shell comprises at least one polymer; and at least one boron cluster. In some embodiments, the composition further comprises at least one targeting moiety. In some embodiments, the targeting moiety is attached to the shell.

In various embodiments, the present invention provides an article of manufacture, comprising: a core, wherein the core comprises at least one iron oxide; a shell surrounding the core, wherein the shell comprises at least one polymer; at least one boron cluster; and at least one targeting moiety attached to the shell. In some embodiments, the article of manufacture is a nanoparticle. In some embodiments, at least one boron cluster is encapsulated in the at least one polymer. In some embodiments, at least one boron cluster is linked to the at least one polymer.

In various embodiments, the present invention provides an article of manufacture, comprising: a core, wherein the core comprises at least one iron oxide; a shell surrounding the core, wherein the shell comprises at least one polymer; and at least one boron cluster. In some embodiments, the article of manufacture further comprises at least one targeting moiety. In some embodiments, the targeting moiety is attached to the shell.

In various embodiments, the present invention provides a nanoparticle, comprising: ferumoxytol; at least one boron cluster; and at least one targeting moiety. In some embodiments, the ferumoxytol comprises carboxymethyl dextran. In some embodiments, at least one boron cluster is encapsulated in the carboxymethyl dextran. In some embodiments, at least one boron cluster is linked to the carboxymethyl dextran. In some embodiments, at least one boron cluster is encapsulated in the ferumoxytol. In some embodiments, at least one boron cluster is linked to the ferumoxytol.

In various embodiments, the present invention provides a nanoparticle, comprising: ferumoxytol; and at least one boron cluster. In some embodiments, the nanoparticle further comprises at least one targeting moiety.

In various embodiments, the present invention provides a nanoparticle, comprising at least one selected from the group consisting of Ferumoxytol, Ferumoxides, Ferucarbotran, Ferumoxtran-10, NC100150, VSOP C184, and combinations thereof; at least one boron cluster; and at least one targeting moiety. In various embodiments, the present invention provides a composition, comprising at least one selected from the group consisting of Ferumoxytol, Ferumoxides, Ferucarbotran, Ferumoxtran-10, NC100150, VSOP C184, and combinations thereof; at least one boron cluster; and at least one targeting moiety. In various embodiments, the present invention provides an article of manufacture, comprising at least one selected from the group consisting of Ferumoxytol, Ferumoxides, Ferucarbotran, Ferumoxtran-10, NC100150, VSOP C184, and combinations thereof; at least one boron cluster; and at least one targeting moiety.

In various embodiments, the present invention provides a nanoparticle, composition, or article of manufacture comprising at least one selected from the group consisting of Ferumoxytol, Ferumoxides, Ferucarbotran, Ferumoxtran-10, NC100150, VSOP C184, and combinations thereof; and at least one boron cluster. In some embodiments, the nanoparticle, composition, or article of manufacture further comprises at least one targeting moiety.

In various embodiments, the present invention provides a nanoparticle, comprising: a core; a coating surrounding the core; at least one boron cluster; and at least one targeting moiety. In various embodiments, the present invention provides a composition, comprising: a core; a coating surrounding the core; at least one boron cluster; and at least one targeting moiety. In various embodiments, the present invention provides an article of manufacture, comprising: a core; a coating surrounding the core; at least one boron cluster; and at least one targeting moiety.

In various embodiments, the present invention provides a nanoparticle, composition, or article of manufacture comprising: a core; a coating surrounding the core; and at least one boron cluster. In some embodiments, the nanoparticle, composition, or article of manufacture further comprises at least one targeting moiety.

In various embodiments, the present invention provides a nanoparticle, comprising coated iron oxide or a coated iron oxide particle; at least one boron cluster; and at least one targeting moiety.

In various embodiments, the present invention provides a nanoparticle, composition, or article of manufacture, comprising coated iron oxide or a coated iron oxide particle; and at least one boron cluster. In some embodiments, the nanoparticle, composition, or article of manufacture further comprises at least one targeting moiety.

In some embodiments, the coated iron oxide particle is selected from the group consisting of Ferumoxytol, Ferumoxides, Ferucarbotran, Ferumoxtran-10, NC100150, VSOP C184, and combinations thereof. In some embodiments, the coated iron oxide is selected from the group consisting of Ferumoxytol, Ferumoxides, Ferucarbotran, Ferumoxtran-10, NC100150, VSOP C184, and combinations thereof. In some embodiments, the coating comprises at least one selected from the group consisting of polymer, co-polymer, monomer, and combinations thereof.

In some embodiments, the shell comprises at least one selected from the group consisting of polymer, co-polymer, monomer, and combinations thereof.

In some embodiments, the core comprises at least one iron oxide.

In some embodiments, the nanoparticle optionally further comprises at least one drug.

In some embodiments, the nanoparticle optionally further comprises at least one fluorescent dye.

In some embodiments, the nanoparticle is a multimodal probe. In some embodiments, the nanoparticle is a multimodal nanoparticle. In some embodiments, the nanoparticle may be used for multimodal detection of a cancer in a subject. In some embodiments, the nanoparticle may be used for multimodal detection of a tumor in a subject. In some embodiments, the nanoparticle may be used for multimodal detection of a tumor margin of a tumor in a subject. In some embodiments, the nanoparticle may be used to deliver a boron cluster for example to a cancer cell, cancer tissue, cancerous cell, cancerous tissue, or tumor.

In some embodiments, the nanoparticles of the present invention may be used to determine tumor concentration in a subject. In some embodiments, the nanoparticles of the present invention may be for dual visualization by magnetic resonance imaging (MRI) and fluorescence imaging. In some embodiments, the nanoparticles of the present invention may be used as markers during fluorescence image guided surgery for the intraoperative detection of tumor margins. In some embodiments, the nanoparticles of the present invention may be used to visualize nanoparticle delivery or boron cluster delivery by magnetic resonance imaging and/or fluorescence imaging. In some embodiments, the fluorescence imaging is selected from the group consisting of near infrared fluorescence imaging, intraoperative fluorescence imaging, and combinations thereof.

Nanoparticles of the present invention may be administered to a subject (and thereby contacted with a tissue), or contacted with a tissue in vivo or in vitro. Thus, in some embodiments, the methods are applicable to both human therapy and veterinary applications, as well as research applications in vitro or within animal models.

In some embodiments, the nanoparticles of the present invention further comprise at least one drug.

In some embodiments, the compositions of the present invention further comprise at least one drug.

In some embodiments, the articles of manufacture of the present invention further comprise at least one drug.

In some embodiments, the nanoparticles of the present invention selectively target and/or bind to diseased tissue and/or diseased cells. In some embodiments, the nanoparticles of the present invention selectively target and/or bind to cancerous tissue, cancer tissue, cancer cells, tumor, tumor tissue, tumor cells, and combinations thereof.

In some embodiments, the nanoparticles selectively targets and/or binds to diseased tissue and/or diseased cells compared to non-diseased tissue and/or non-diseased cells. In some embodiments, the nanoparticles selectively targets and/or binds to cancerous tissue, cancer tissue, cancer cells, tumor, tumor tissue, tumor cells, and combinations thereof compared to healthy tissue, non-cancererous tissue, non-cancerous cells.

Iron Oxide Particles

Feraheme (FH), also known as Ferumoxytol, is an FDA-approved carboxymethyl dextran coated iron oxide nanoparticle formulation for the treatment of anemia. Feraheme (FH) is also used off-label as an MRI contrast agent. In various embodiments, Feraheme (FH) can be modified with targeting moieties to facilitate receptor mediated tumor accumulation or permeability through the brain blood barrier.

Non-limiting examples of coated iron oxide and/or coated iron oxide particles include Ferumoxytol (Feraheme®), Ferumoxides (Feridex® IV, Berlex Laboratories), Ferucarbotran (Resovist®, Bayer Healthcare), Ferumoxtran-10 (AI-227 or Code-7227, Combidex®, AMAG Pharma; Sinerem®, Guerbet), NC100150 (Clariscan®, Nycomed) and (VSOP C184, Ferropharm).

In some embodiments, the at least one coated iron oxide and/or at least one coated iron oxide particle is selected from the group consisting of Ferumoxytol, Ferumoxides, Ferucarbotran, Ferumoxtran-10, NC100150, VSOP C184, and combinations thereof.

In some embodiments, the iron oxide is superparamagnetic iron oxide (SPIO).

Polymers

In some embodiments, the at least one polymer is at least one biocompatible polymer.

In some embodiments, the at least one polymer is at least one polysaccharide.

In some embodiments, the at least one polymer is one selected from the group consisting of at least one dextran, at least one unfunctionalized dextran, at least one functionalized dextran, at least one unsubstituted dextran, at least one substituted dextran, and combinations thereof.

In some embodiments, the at least one polymer is selected from the group consisting of carboxymethyl dextran, at least one dextran, and combinations thereof.

In some embodiments, the at least one polymer is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In some embodiments, the at least one polymer is poly(acrylic acid) (PAA).

Polysaccharides

In various embodiments, the at least one polymer is at least one polysaccharide.

In various embodiments, the at least one polysaccharide is selected from at least one dextran, at least one unfunctionalized dextran, at least one functionalized dextran, at least one unsubstituted dextran, at least one substituted dextran, and combinations thereof.

In some embodiments, the at least one polysaccharide is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

Dextrans

Dextrans are polysaccharides which have a linear backbone of α-linked d-glucopyranosyl repeating units. Three classes of dextrans can be differentiated by their structural features. The pyranose ring structure contains five carbon atoms and one oxygen atom. Class 1 dextrans contain the α(1→6)-linked d-glucopyranosyl backbone modified with small side chains of d-glucose branches with α(1→2), α(1→3), and α(1→4)-linkage. The class 1 dextrans vary in their molecular weight, spatial arrangement, type and degree of branching, and length of branch chains depending on the microbial producing strains and cultivation conditions. Isomaltose and isomaltotriose are oligosaccharides with the class 1 dextran backbone structure. Class 2 dextrans (alternans) contain a backbone structure of alternating α(1-3) and α(1→6)-linked d-glucopyranosyl units with α(1→3)-linked branches. Class 3 dextrans (mutans) have a backbone structure of consecutive α(1→3)-linked d-glucopyranosyl units with α(1→6)-linked branches.

In various embodiments, the at least one polymer is selected from the group consisting at least one dextran, at least one unfunctionalized dextran, at least one functionalized dextran, at least one unsubstituted dextran, at least one substituted dextran, and combinations thereof.

In various embodiments, the at least one polymer is selected from the group consisting of at least one dextran, carboxymethyl dextran, and combinations thereof.

In various embodiments, the at least one polymer is carboxymethyl dextran.

In some embodiments, the at least one dextran is selected from the group consisting of a class 1 dextran, a class 2 dextran, a class 3 dextran, and combinations thereof.

Probes

In various embodiments, the present invention provides a probe comprising a coated iron oxide nanoparticle; at least one boron cluster; and at least one targeting moiety. In some embodiments, the at least one targeting moiety is attached to the coated iron oxide nanoparticle. In some embodiments, the coated iron oxide nanoparticle is selected from the group consisting of Ferumoxytol, Ferumoxides, Ferucarbotran, Ferumoxtran-10, NC100150, VSOP C184, and combinations thereof.

In various embodiments, the present invention provides a probe comprising a coated iron oxide nanoparticle; and at least one boron cluster. In some embodiments, the probe further comprises at least one targeting moiety. In some embodiments, the at least one targeting moiety is attached to the coated iron oxide nanoparticle. In some embodiments, the coated iron oxide nanoparticle is selected from the group consisting of Ferumoxytol, Ferumoxides, Ferucarbotran, Ferumoxtran-10, NC100150, VSOP C184, and combinations thereof.

In some embodiments, the probe further comprises at least one drug. In some embodiments, the probe further comprises at least one fluorescent dye. In some embodiments, the probe further comprises at least one drug, and at least one fluorescent dye.

In some embodiments, the probe is a multimodal probe. In some embodiments, the probe may be used for multimodal detection of a cancer in a subject. In some embodiments, the probe may be used for multimodal detection of a tumor in a subject. In some embodiments, the probe may be used for multimodal detection of a tumor margin of a tumor in a subject. In some embodiments, the probe may be used to deliver a boron cluster for example to a cancer cell, cancer tissue, cancerous cell, cancerous tissue, or tumor. In some embodiments, the probe may be used to deliver a boron cluster and a drug for example to a cancer cell, cancer tissue, cancerous cell, cancerous tissue, or tumor.

In some embodiments, the probes of the present invention may be used to determine tumor concentration in a subject. In some embodiments, the probes of the present invention may be for dual visualization by magnetic resonance imaging (MRI) and fluorescence imaging. In some embodiments, the probes of the present invention may be used as markers during fluorescence image guided surgery for the intraoperative detection of tumor margins. In some embodiments, the probes of the present invention may be used to visualize boron cluster delivery by magnetic resonance imaging and/or fluorescence imaging. In some embodiments, the probes of the present invention may be used to visualize boron cluster and a drug delivery by magnetic resonance imaging and/or fluorescence imaging. In some embodiments, the fluorescence imaging is selected from the group consisting of near infrared fluorescence imaging, intraoperative fluorescence imaging, and combinations thereof.

Probes of the present invention may be administered to a subject (and thereby contacted with a tissue), or contacted with a tissue in vivo or in vitro. Thus, in some embodiments, the methods are applicable to both human therapy and veterinary applications, as well as research applications in vitro or within animal models.

In some embodiments, the probes of the present invention selectively target and/or bind to diseased tissue and/or diseased cells. In some embodiments, the probes of the present invention selectively target and/or bind to cancerous tissue, cancer tissue, cancer cells, tumor, tumor tissue, tumor cells, and combinations thereof.

In some embodiments, the probes selectively targets and/or binds to diseased tissue and/or diseased cells compared to non-diseased tissue and/or non-diseased cells. In some embodiments, the probes selectively targets and/or binds to cancerous tissue, cancer tissue, cancer cells, tumor, tumor tissue, tumor cells, and combinations thereof compared to healthy tissue, non-cancererous tissue, non-cancerous cells.

Targeting Moiety

The terms “targeting moiety” and “targeting agent” and “targeting ligand” are used interchangeably herein and are intended to mean any agent, such as for example a molecule, compound, or peptide, that serves to target or direct the nanoparticle or probe to a particular location or association (e.g., a specific binding event). Thus, for example, a targeting moiety may be used to target a molecule to a specific target protein or enzyme, or to a particular cellular location, or to a particular cell type, to selectively enhance accumulation of the nanoparticle or probe. For example, as discussed more fully herein, the nanoparticles and probes of the present invention include a targeting moiety to target the nanoparticles and probes to a specific cell type such as tumor cells, such as a transferrin moiety, since many tumor cells have significant transferrin receptors on their surfaces. Similarly, a targeting moiety may include components useful in targeting the nanoparticles or probes to a particular subcellular location. As will be appreciated by those of in the art, the localization of proteins within a cell is a simple method for increasing effective concentration. For example, shuttling a boron cluster or a boron cluster and an optional drug into the nucleus confines them to a smaller space thereby increasing concentration. The physiological target may simply be localized to a specific compartment, and the agent must be localized appropriately. More than one targeting moiety can be linked, connected, conjugated, attached, or otherwise associated with each nanoparticle or probe, and the target molecule for each targeting moiety can be the same or different.

The targeting moiety can function to target or direct the nanoparticle or probe to a particular location, cell type, tissue type, diseased cell, diseased tissue, or association. In general, the targeting moiety is directed against a target molecule. As will be appreciated by those in the art, the nanoparticles of the invention or probes of the invention are can be applied locally or systemically administered (e.g., injected intravenously).

In some embodiments, the targeting moiety may be used to either allow the internalization of the nanoparticle or probe to the cell cytoplasm or localize it to a particular cellular compartment, such as the nucleus. In some embodiments, the targeting moiety allows targeting of the nanoparticles of the present invention or probes of the present invention to a particular subcellular location, for example, the cytoplasm, Golgi, endoplasmic reticulum, nucleus, nucleoli, nuclear membrane, mitochondria, secretory vesicles, lysosome, and cellular membrane.

In some embodiments, the targeting moiety allows targeting of the nanoparticles of the present invention or probes of the present invention to extracellular locations (e.g., via a secretory signal). In some embodiments, the targeting moiety allows targeting of the nanoparticles of the present invention or probes of the present invention to a particular tissue or the surface of a cell (e.g., tumor tissue, cancer tissue, tumor cell, cancer cell). That is, in some embodiments, the nanoparticles of the present invention or probes of the present invention need not be taken up into the cytoplasm of a cell to be activated.

In some embodiments, the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep-2, modified angiopep-2, unsubstituted angiopep-2, substituted angiopep-2, unfunctionalized angiopep-2, functionalized angiopep-2, and combinations thereof.

In some embodiments, the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof. In some embodiments, the angiopep is selected from the group consisting of angiopep-1, angiopep-2, angiopep-5, angiopep-7, and combinations thereof. In some embodiments, the modified angiopep is selected from the group consisting of modified angiopep-1, modified angiopep-2, modified angiopep-5, modified angiopep-7, and combinations thereof. In some embodiments, the unsubstituted angiopep is selected from the group consisting of unsubstituted angiopep-1, unsubstituted angiopep-2, unsubstituted angiopep-5, unsubstituted angiopep-7, and combinations thereof. In some embodiments, the substituted angiopep is selected from the group consisting of substituted angiopep-1, substituted angiopep-2, substituted angiopep-5, unsubstituted angiopep-7, and combinations thereof. In some embodiments, unfunctionalized angiopep is selected from the group consisting of unfunctionalized angiopep-1, unfunctionalized angiopep-2, unfunctionalized angiopep-5, unfunctionalized angiopep-7, and combinations thereof. In some embodiments, functionalized angiopep is selected from the group consisting of functionalized angiopep-1, functionalized angiopep-2, functionalized angiopep-5, functionalized angiopep-7, and combinations thereof.

In some embodiments, the targeting moiety is selected from the group consisting of heptamethine carbocyanine dye, modified heptamethine carbocyanine dye, unsubstituted heptamethine carbocyanine dye, substituted heptamethine carbocyanine dye, unfunctionalized heptamethine carbocyanine dye, functionalized heptamethine carbocyanine dye, and combinations thereof.

In some embodiments, the targeting moiety is selected from the group consisting of heptamethine cyanine dye, modified heptamethine cyanine dye, unsubstituted heptamethine cyanine dye, substituted heptamethine cyanine dye, unfunctionalized heptamethine cyanine dye, functionalized heptamethine cyanine dye, and combinations thereof.

In some embodiments, the targeting moiety is a compound selected from the group consisting of Formula I and Formula II:

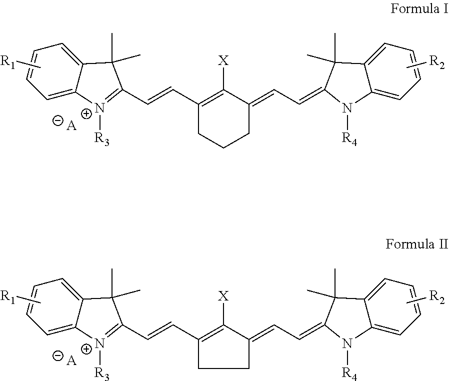

wherein R₁ and R₂ are each independently selected from the group consisting of hydrogen, sulfonato, an electron withdrawing group (EWG), an electron donating group (EDG), and are each independently attached at any of the aromatic ring positions;

R₃ and R₄ are independently selected from the group consisting of hydrogen, alkyl, aryl, aralkyl, alkylsulfonato, alkylcarboxy, alkylcarboxyl, alkylamino, ω-alkylaminium, ω-alkynyl, PEGyl, PEGylcarboxylate, ω-PEGylaminium, ω-acyl-NHR₅, and ω-acyl-lysine, wherein R₅ is selected from the group consisting of hydrogen and alkyl;

X is selected from the group consisting of hydrogen, halogen, CN, Me, OH, 4-O-Ph-CH₂CH₂COOH, 4-O-Ph-NHR₆, NHR₇, 4-S-Ph-NHR₈, ω-iminoacyl-NHR₉, and ω-aminoacyl-lysine, wherein R₆, R₇, R₈, and R₉ are each independently selected from the group consisting of hydrogen and alkyl; and counteranion A is selected from the group consisting of iodide, bromide, arylsulfonato, alkylsulfonato, tetrafluoroborate, chloride, and a pharmaceutically acceptable anion.

In some embodiments, R₃ and R₄ are not both hydrogen. In some embodiments, the counteranion A is not present.

In some embodiments, the targeting moiety targets glioma. In some embodiments, the targeting moiety is a glioma targeting moiety.

The term “modified” refers to an alteration from an entity's normally occurring state. An entity can be modified by removing discrete chemical units or by adding discrete chemical units.

The terms “linked”, “joined”, “grafted”, “tethered”, “associated”, “attached”, “connected” and “conjugated” in the context of the nanovehicles of the invention or nanoparticles of the invention or probes of the invention or compositions of the invention or articles of manufacture of the invention, are used interchangeably to refer to any method known in the art for functionally connecting moieties (e.g., targeting moieties, targeting ligands, etc.) to the nanovehicles, nanoparticles, probes, compositions, articles of manufacture or components thereof or to the coated iron oxide nanoparticles or components thereof, including, without limitation, recombinant fusion, covalent bonding, non-covalent bonding, disulfide bonding, ionic bonding, hydrogen bonding, and electrostatic bonding.

In various embodiments, the at least one targeting moiety is attached to the at least one polymer or the ferumoxytol. In some embodiments, the at least one targeting moiety is linked to the at least one polymer or the ferumoxytol by at least one linkage. In some embodiments, the at least one targeting moiety is linked to the at least one polymer or the ferumoxytol by at least one linker. In some embodiments, the at least one targeting moiety is linked to the at least one carboxymethyl dextran. In some embodiments, the at least one targeting moiety is linked to the carboxymethyl dextran by at least one linkage. In some embodiments, the at least one targeting moiety is linked to the carboxymethyl dextran by at least one linker. In some embodiments, the at least one targeting moiety is attached to the shell. In some embodiments, the at least one targeting moiety is attached to the shell of the nanoparticle or probe. In some embodiments, the at least one targeting moiety is attached to the shell of the nanoparticle or probe by at least one linkage.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

In some embodiments, the targeting moiety selectively targets and/or binds to diseased tissue and/or diseased cells. In some embodiments, the targeting moiety of the present invention selectively targets and/or binds to cancerous tissue, cancer tissue, cancer cells, tumor, tumor tissue, tumor cells, and combinations thereof.

In some embodiments, the targeting moiety selectively targets and/or binds to diseased tissue and/or diseased cells compared to non-diseased tissue and/or non-diseased cells. In some embodiments, the targeting moiety selectively targets and/or binds to cancerous tissue, cancer tissue, cancer cells, tumor, tumor tissue, tumor cells, and combinations thereof compared to healthy tissue, non-cancererous tissue, non-cancerous cells.

In some embodiments, the targeting moiety is an antibody that selectively targets and/or binds to diseased tissue and/or diseased cells compared to non-diseased tissue and/or non-diseased cells. In some embodiments, targeting moiety is an antibody that selectively targets and/or binds to cancerous tissue, cancer tissue, cancer cells, tumor, tumor tissue, tumor cells, and combinations thereof compared to healthy tissue, non-cancererous tissue, non-cancerous cells.

In some embodiments, the targeting moiety is a peptide that selectively targets and/or binds to diseased tissue and/or diseased cells compared to non-diseased tissue and/or non-diseased cells. In some embodiments, the targeting moiety is a peptide that selectively targets and/or binds to cancerous tissue, cancer tissue, cancer cells, tumor, tumor tissue, tumor cells, and combinations thereof compared to healthy tissue, non-cancererous tissue, non-cancerous cells.

Drugs

In various embodiments, the nanoparticles, compositions, articles of manufacture, and/or probes of the present invention may optionally further comprise at least one drug loaded into or encapsulated into or attached to the nanoparticles, compositions, articles of manufacture, and/or probes or components thereof. In various embodiments, the nanoparticle further comprises at least one drug. In various embodiments, the probe further comprises at least one drug.

In some embodiments, the at least one drug is encapsulated in the nanoparticle. In some embodiments, the at least one drug is encapsulated in the at least one polymer or in the ferumoxytol. In some embodiments, the at least one drug is linked to the at least one polymer or to the ferumoxytol. In some embodiments, the at least one drug is linked to the at least one polymer or to the ferumoxytol by at least one linkage. In some embodiments, the at least one drug is linked to the at least one polymer or to the ferumoxytol by at least one linker. In some embodiments, at least one drug is encapsulated in the carboxymethyl dextran. In some embodiments, the at least one drug is linked to the at least one carboxymethyl dextran. In some embodiments, the at least one drug is linked to the carboxymethyl dextran by at least one linkage. In some embodiments, the at least one drug is linked to the carboxymethyl dextran by at least one linker. In some embodiments, the at least one drug is encapsulated in the at least one coated iron oxide nanoparticle. In some embodiments, the at least one drug is linked to the at least one coated iron oxide nanoparticle. In some embodiments, the at least one drug is linked to the at least one coated iron oxide nanoparticle by at least one linkage. In some embodiments, the at least one drug is linked to the at least one coated iron oxide nanoparticle by at least one linker. In some embodiments, at least one drug is encapsulated in the shell.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

As used herein, the term “drug” refers to any agent capable of having a physiologic effect (e.g., a therapeutic or prophylactic effect) on a biosystem such as a prokaryotic or eukaryotic cells, or prokaryotic or eukaryotic tissue, or a subject (e.g., a patient), in vivo or in vitro, including, without limitation, chemotherapeutics, toxins, radiotherapeutics, radiosenitizing agents, gene therapy vectors, antisense nucleic acid constructs, transcription factor decoys, imaging agents, diagnostic agents, agents known to interact with an intracellular protein, polypeptides, and polynucleotides. Drugs that may be utilized in the nanovehicles or nanoparticles or probes include any type of compound including antibacterial, antiviral, antifungal, or anti-cancer agents. In some embodiments, the drug may be modified to attach a polymerizable moiety. In some embodiments, the drug is water-insoluble, poorly water soluble, or water-soluble. In some embodiments, the drug is a solid or liquid. In some embodiments, the drug is a therapeutic agent. In some embodiments, the drug is not a therapeutic agent.

The drug need not be a therapeutic agent. For example, the drug may be cytotoxic to the local cells or tissue to which it is delivered but have an overall beneficial effect on the subject. Further, the drug may be a diagnostic agent with no direct therapeutic activity per se, such as a contrast agent for bioimaging.

As used herein, the term “therapeutic agent” refers to a compound used to treat or prevent a disease, disorder, disease condition in a subject so as to provide a therapeutic benefit to the subject. In some embodiments, the therapeutic agent is administered to the subject in a therapeutically effective amount.

A description of various classes of drugs and diagnostic agents and a listing of species within each class can be found, for instance, in Martindale, The Extra Pharmacopoeia, Twenty-ninth Edition (The Pharmaceutical Press, London, 1989), which is incorporated herein by reference in its entirety. The drugs or diagnostic agents are commercially available and/or can be prepared by techniques known in the art.

Non-limiting examples of drugs include analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocling agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

Non-limiting examples of drugs include analgesics, anesthetics, anti-inflammatory agents, anthelmintics, anti-arrhythmic agents, antiasthma agents, antibiotics, anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives, astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants, diagnostic agents, diagnostic imaging agents, diuretics, dopaminergics, free radical scavenging agents, growth factors, haemostatics, immunological agents, lipid regulating agents, muscle relaxants, proteins, peptides and polypeptides xanthines, alprazolam, amiodarone, amlodipine, astemizole, atenolol, azathioprine, azelatine, beclomethasone, β-lactam, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyridamole, divalproex, dobutamine, doxazosin, enalapril, estradiol, etodolac, etoposide, famotidine, felodipine, fentanyl citrate, fexofenadine, finasteride, fluconazole, flunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, penicillin, phenytoin, piroxicam, quinapril, ramipril, risperidone, sertraline, simvastatin, steroids, taxol, terbinafine, terfenadine, triamcinolone, valproic acid, zolpidem, expectorants, mucolytics, hypnotics, neuroleptics, and a pharmaceutically acceptable salt of any of the foregoing.

Non-limiting examples of drugs include alprazolam, amiodarone, amlodipine, astemizole, atenolol, azathioprine, azelatine, beclomethasone, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyridamole, divalproex, dobutamine, doxazosin, enalapril, estradiol, etodolac, etoposide, famotidine, felodipine, fentanyl citrate, fexofenadine, finasteride, fluconazole, fiunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, phenytoin, piroxicam, quinapril, ramipril, risperidone, sertraline, simvastatin, sulindac, terbinafine, terfenadine, triamcinolone, valproic acid, zolpidem, or pharmaceutically acceptable salts of any of the above-mentioned drugs.

Non-limiting examples of drugs include cisplatin, carboplatin, oxaliplatin, bortezomib, camptothecin, topotecan, irinotecan, temozolomide, doxorubicin, etoposide or pharmaceutically acceptable salts of any of the above-mentioned drugs.

In some embodiments, the drug is selected from the group consisting of docetaxel (DXT), paclitaxel (PXT), bortezomib (Bort), cobozentanib (cabo), brefeldin A (BFA), boron clusters, and combinations thereof.

In some embodiments, the drug is a boron cluster.

In some embodiments, the nanoparticles of the present invention can be used to deliver a boron cluster and an optional drug that is cytotoxic to cancer cells or tumor cells. In some embodiments, the probes of the present invention can be used to deliver a boron cluster and an optional drug that is cytotoxic to cancer cells or tumor cells.

The terms “linked”, “joined”, “grafted”, “tethered”, “associated”, “attached”, “connected” and “conjugated” in the context of the nanovehicles of the invention or nanoparticles of the invention or the probes of the invention or compositions of the invention or articles of manufacture of the invention, are used interchangeably to refer to any method known in the art for functionally connecting drugs to the nanovehicles, nanoparticles, probes, compositions, articles of manufacture or components thereof or the coated iron oxide nanoparticles or components thereof, including, without limitation, recombinant fusion, covalent bonding, non-covalent bonding, disulfide bonding, ionic bonding, hydrogen bonding, and electrostatic bonding.

Fluorescent Dyes

In various embodiments, the nanoparticles, compositions, articles of manufacture, and/or probes of the present invention may optionally further comprise at least one fluorescent dye loaded into or encapsulated into or attached to the nanoparticles, compositions, articles of manufacture, and/or probes or components thereof. In various embodiments, the nanoparticle further comprises at least one fluorescent dye.

In some embodiments, the at least one fluorescent dye is encapsulated in the nanoparticle. In some embodiments, the at least one fluorescent dye is encapsulated in the at least one polymer or in the ferumoxytol. In some embodiments, the at least one fluorescent dye is linked to the at least one polymer or to the ferumoxytol. In some embodiments, the at least one fluorescent dye is linked to the at least one polymer or to the ferumoxytol by at least one linkage. In some embodiments, the at least one fluorescent dye is linked to the at least one polymer or to the ferumoxytol by at least one linker. In some embodiments, at least one fluorescent dye is encapsulated in the carboxymethyl dextran. In some embodiments, the at least one fluorescent dye is linked to the at least one carboxymethyl dextran. In some embodiments, the at least one fluorescent dye is linked to the carboxymethyl dextran by at least one linkage. In some embodiments, the at least one fluorescent dye is linked to the carboxymethyl dextran by at least one linker. In some embodiments, at least one fluorescent dye is encapsulated in the shell.

In some embodiments, the at least one fluorescent dye is encapsulated in the at least one coated iron oxide nanoparticle. In some embodiments, the at least one fluorescent dye is linked to the at least one coated iron oxide nanoparticle. In some embodiments, the at least one fluorescent dye is linked to the at least one coated iron oxide nanoparticle by at least one linkage. In some embodiments, the at least one fluorescent dye is linked to the at least one coated iron oxide nanoparticle by at least one linker.

In some embodiments, the fluorescent dye is a near infrared dye. In some embodiments, the fluorescent dye is a near infrared fluorescent dye.

Non-limiting examples of fluorescent dyes include DiI, DiR, heptamethine cyanine (HMC), IR820, or combinations thereof.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

The terms “linked”, “joined”, “grafted”, “tethered”, “associated”, “attached”, “connected” and “conjugated” in the context of the nanovehicles of the invention or nanoparticles of the invention or the probes of the invention or compositions of the invention or articles of manufacture of the invention, are used interchangeably to refer to any method known in the art for functionally connecting fluorescent dyes to the nanovehicles, nanoparticles, probes, compositions, articles of manufacture or components thereof or the coated iron oxide nanoparticles or components thereof, including, without limitation, recombinant fusion, covalent bonding, non-covalent bonding, disulfide bonding, ionic bonding, hydrogen bonding, and electrostatic bonding.

Pharmaceutical Compositions

In various embodiments the present invention also provides the nanoparticles of the present invention in the form of various pharmaceutical formulations. The present invention also provides the probes of the present invention in the form of various pharmaceutical formulations. These pharmaceutical compositions may be used for example for detecting, diagnosing, treating, detecting and treating, diagnosing and treating, reducing the severity of and/or slowing the progression of a disease, disorder, or disease condition in a subject. In accordance with the invention, the disease, disorder, or disease condition can be a cancer.

In various embodiments, the present invention provides a pharmaceutical composition comprising at least one nanoparticle described herein. In another embodiment, the present invention provides a pharmaceutical composition comprising at least two nanoparticles described herein. In still another embodiment, the present invention provides a pharmaceutical composition comprising a plurality of nanoparticles described herein. In various embodiments, the pharmaceutical compositions also exhibit minimal toxicity when administered to a mammal.

In various embodiments, the present invention provides a pharmaceutical composition comprising at least one probe described herein. In another embodiment, the present invention provides a pharmaceutical composition comprising at least two probes described herein. In still another embodiment, the present invention provides a pharmaceutical composition comprising a plurality of probes described herein. In various embodiments, the pharmaceutical compositions also exhibit minimal toxicity when administered to a mammal.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In certain embodiments, the pharmaceutical composition is formulated for intravascular, intravenous, intraarterial, intratumoral, intramuscular, subcutaneous, intranasal, intraperitoneal, or oral administration.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

Kits

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least one nanoparticle of the present invention described herein; and instructions for using the nanoparticles to detect, diagnose, treat, detect and treat, diagnose and treat, reduce the severity of and/or slow the progression of the disease, disorder, or disease condition in the subject. In some embodiments, the nanoparticle comprises at least one fluorescent dye.

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least one probe of the present invention described herein; and instructions for using the probes to detect, diagnose, treat, detect and treat, diagnose and treat, reduce the severity of and/or slow the progression of the disease, disorder, disease condition in the subject. In some embodiments, the probe comprises at least one fluorescent dye.

The kit is an assemblage of materials or components, including at least one of the inventive compositions and/or nanoparticles and/or probes. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Methods for Detecting Nanoparticles

In various embodiments, the present invention provides a method for detecting at least one nanoparticle in a subject, comprising: administering the at least one nanoparticle to the subject; and detecting the at least one nanoparticle in the subject by an imaging method. In some embodiments, the at least one nanoparticle is a nanoparticle of the present invention.

In various embodiments the present invention provides a method for detecting at least one nanoparticle in a subject, comprising: administering the at least one nanoparticle to the subject, thereby contacting a tissue of the subject with the at least one nanoparticle such that the at least one nanoparticle binds to the tissue; and detecting the at least one nanoparticle bound to the tissue by an imaging method. In some embodiments, the at least one nanoparticle is a nanoparticle of the present invention.

In some embodiments, the imaging method is selected from the group consisting of magnetic resonance imaging, fluorescence imaging, and combinations thereof. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof.

In some embodiments, the imaging method comprises operating an imaging scanner. In some embodiments, the imaging method comprises operating an imaging machine. In some embodiments, the imaging method comprises operating imaging equipment.

In some embodiments, the magnetic resonance imaging comprises operating a magnetic resonance imaging scanner. In some embodiments, the magnetic resonance imaging comprises operating a magnetic resonance imaging machine. In some embodiments, the magnetic resonance imaging comprises operating a magnetic resonance imaging instrument.

In some embodiments, the fluorescence imaging comprises operating a fluorescence imaging scanner. In some embodiments, the fluorescence imaging comprises operating a fluorescence imaging machine. In some embodiments, the fluorescence imaging comprises operating a fluorescence imaging instrument.

In some embodiments, the near infrared fluorescence imaging comprises operating a near infrared fluorescence imaging scanner or a fluorescence imaging scanner. In some embodiments, the near infrared fluorescence imaging comprises operating a near infrared fluorescence imaging machine or a fluorescence imaging machine. In some embodiments, the near infrared fluorescence imaging comprises operating a near infrared fluorescence imaging instrument or a fluorescence imaging instrument.

In some embodiments, the intraoperative fluorescence imaging comprises operating an intraoperative fluorescence imaging scanner or a fluorescence imaging scanner. In some embodiments, the intraoperative fluorescence imaging comprises operating an intraoperative fluorescence imaging machine or a fluorescence imaging machine. In some embodiments, the intraoperative fluorescence imaging comprises operating an intraoperative fluorescence imaging instrument or a fluorescence imaging instrument.

In some embodiments, the tissue is selected from the group consisting of cancerous tissue, cancer tissue, tumor, tumor tissue, and combinations thereof.

Methods for Detecting Probes

In various embodiments, the present invention provides a method for detecting at least one probe in a subject, comprising: administering the at least one probe to the subject; and detecting the at least one probe in the subject by an imaging method. In some embodiments, the at least one probe is a probe of the present invention.

In various embodiments the present invention provides a method for detecting at least one probe in a subject, comprising: administering the at least one probe to the subject, thereby contacting a tissue of the subject with the at least one probe such that the at least one probe binds to the tissue; and detecting the at least one probe bound to the tissue by an imaging method. In some embodiments, the at least one probe is a probe of the present invention.

In some embodiments, the imaging method is selected from the group consisting of magnetic resonance imaging, fluorescence imaging, and combinations thereof. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof.

In some embodiments, the tissue is selected from the group consisting of cancerous tissue, cancer tissue, tumor, tumor tissue, and combinations thereof. In some embodiments, the tissue is selected from the group consisting of non-cancerous tissue, healthy tissue, normal tissue, cancerous tissue, cancer tissue, tumor, tumor tissue, and combinations thereof. In some embodiments, the tissue is selected from the group consisting of non-diseased tissue, healthy tissue, normal tissue, diseased tissue, and combinations thereof.

Methods for Diagnosing Cancer

In various embodiments, the present invention provides a method for diagnosing a cancer in a subject, comprising: administering an effective amount of at least one nanoparticle to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject. In some embodiments, the at least one nanoparticle is a nanoparticle of the present invention.

In various embodiments, the present invention provides a method for diagnosing a cancer in a subject, comprising: administering an effective amount of at least one probe to the subject, thereby contacting a tissue of the subject with the at least one probe such that the at least one probe binds to the tissue; and detecting the at least one probe bound to the tissue, wherein the presence of the at least one probe bound to the tissue is indicative of the cancer in the subject. In some embodiments, the at least one probe is a probe of the present invention.

In various embodiments, the present invention provides a method for diagnosing cancer in a subject, comprising: providing at least one nanoparticle, wherein the at least one nanoparticle comprises: a core, wherein the core comprises at least one iron oxide; a shell surrounding the core, wherein the shell comprises at least one polymer; at least one boron cluster; and at least one targeting moiety attached to the shell; administering an effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue; and detecting the at least one nanoparticle bound to the cancerous tissue, wherein the presence of the at least one nanoparticle bound to the cancerous tissue is a diagnosis of the cancer in the subject.

Methods for Detecting Cancer

In various embodiments, the present invention provides a method for detecting a cancer in a subject, comprising: administering an effective amount of at least one nanoparticle to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject. In some embodiments, the at least one nanoparticle is a nanoparticle of the present invention.

In various embodiments, the present invention provides a method for detecting a cancer in a subject, comprising: administering an effective amount of at least one probe to the subject, thereby contacting a tissue of the subject with the at least one probe such that the at least one probe binds to the tissue; and detecting the at least one probe bound to the tissue, wherein the presence of the at least one probe bound to the tissue is indicative of the cancer in the subject. In some embodiments, the at least one probe is a probe of the present invention.

In some embodiments, the tissue is selected from the group consisting of cancerous tissue, cancer tissue, tumor, tumor tissue, and combinations thereof.

In various embodiments, the present invention provides a method for detecting cancer in a subject, comprising: providing at least one nanoparticle, wherein the at least one nanoparticle comprises: a core, wherein the core comprises at least one iron oxide; a shell surrounding the core, wherein the shell comprises at least one polymer; at least one boron cluster; and at least one targeting moiety attached to the shell; administering an effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue; and detecting the at least one nanoparticle bound to the cancerous tissue, wherein the presence of the at least one nanoparticle bound to the cancerous tissue is indicative of the cancer in the subject.

Methods for Treating Cancer

In various embodiments, the present invention provides a method for treating a cancer in a subject, comprising: administering an effective amount of at least one nanoparticle to the subject, thereby contacting a tissue of the subject with the at least one nanoparticle such that the at least one nanoparticle binds to the tissue, wherein the at least one nanoparticle comprises at least one boron cluster; detecting the at least one nanoparticle bound to the tissue, wherein the presence of the at least one nanoparticle bound to the tissue is indicative of the cancer in the subject; and delivering the at least one boron cluster to the tissue thereby treating the cancer in the subject. In some embodiments, the at least one nanoparticle is a nanoparticle of the present invention.

In various embodiments, the present invention provides a method for treating a cancer in a subject, comprising: administering an effective amount of at least one probe to the subject, thereby contacting a tissue of the subject with the at least one probe such that the at least one probe binds to the tissue, wherein the at least one probe comprises at least one boron cluster; detecting the at least one probe bound to the tissue, wherein the presence of the at least one probe bound to the tissue is indicative of the cancer in the subject; and delivering the boron cluster to the tissue thereby treating the cancer in the subject. In some embodiments, the at least one probe is a probe of the present invention.

In some embodiments, the tissue is selected from the group consisting of cancerous tissue, cancer tissue, tumor, tumor tissue, and combinations thereof.

In various embodiments, the present invention provides a method for treating, reducing the severity of and/or slowing the progression of cancer in a subject, comprising: providing at least one nanoparticle, wherein the at least one nanoparticle comprises: a core, wherein the core comprises at least one iron oxide; a shell surrounding the core, wherein the shell comprises at least one polymer; at least one boron cluster; and at least one targeting moiety attached to the shell; and administering a therapeutically effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue, thereby treating, reducing the severity of and/or slowing the progression of the cancer in the subject. In some embodiments, the nanoparticle further comprises at least one drug. In some embodiments, the method further comprises, delivering a drug and a boron cluster, or a boron cluster to the cancerous tissue so as to treat, reduce the severity of and/or slow the progression of the cancer in the subject.

Methods for Diagnosing and Treating Cancer

In various embodiments, the present invention provides a method for diagnosing and treating a cancer in a subject, comprising: administering an effective amount of at least one nanoparticle to the subject, thereby contacting a tissue of the subject with the at least one nanoparticle such that the at least one nanoparticle binds to the tissue, wherein the at least one nanoparticle comprises at least one boron cluster; detecting the nanoparticle bound to the tissue, wherein the presence of the at least one nanoparticle bound to the tissue is indicative of the cancer in the subject; and delivering the boron cluster to the tissue thereby treating the cancer in the subject. In some embodiments, the at least one nanoparticle is a nanoparticle of the present invention.

In various embodiments, the present invention provides a method for diagnosing and treating a cancer in a subject, comprising: administering an effective amount of at least one probe to the subject, thereby contacting a tissue of the subject with the at least one probe such that the at least one probe binds to the tissue, wherein the at least one probe comprises at least one boron cluster; detecting the at least one probe bound to the tissue, wherein the presence of the at least one probe bound to the tissue is indicative of the cancer in the subject; and delivering the boron cluster to the tissue thereby treating the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Detecting and Treating Cancer

In various embodiments, the present invention provides a method for detecting and treating a cancer in a subject, comprising: administering an effective amount of at least one nanoparticle to the subject, thereby contacting a tissue of the subject with the nanoparticle such that the nanoparticle binds to the tissue, wherein the at least one nanoparticle comprises at least one boron cluster; detecting the nanoparticle bound to the tissue, wherein the presence of the nanoparticle bound to the tissue is indicative of the cancer in the subject; and delivering the boron cluster to the tissue thereby treating the cancer in the subject. In some embodiments, the at least one nanoparticle is a nanoparticle of the present invention.

In various embodiments, the present invention provides a method for detecting and treating a cancer in a subject, comprising: administering an effective amount of at least one probe to the subject, thereby contacting a tissue of the subject with the at least one probe such that the at least one probe binds to the tissue, wherein the at least one probe comprises at least one boron cluster; detecting the at least one probe bound to the tissue, wherein the presence of the at least one probe bound to the tissue is indicative of the cancer in the subject; and delivering the boron cluster to the tissue thereby treating the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Reducing the Severity of and/or Slowing the Progression of Cancer

In various embodiments, the present invention provides a method of reducing the severity of and/or slowing the progression of a cancer in a subject, administering an effective amount of at least one nanoparticle to the subject, thereby contacting a tissue of the subject with the nanoparticle such that the nanoparticle binds to the tissue, wherein the at least one nanoparticle comprises at least one boron cluster; detecting the nanoparticle bound to the tissue, wherein the presence of the nanoparticle bound to the tissue is indicative of the cancer in the subject; and delivering the boron cluster to the tissue thereby reducing the severity of and/or slowing the progression of the cancer in the subject. In some embodiments, the at least one nanoparticle is a nanoparticle of the present invention.

In various embodiments, the present invention provides a method of reducing the severity of and/or slowing the progression of a cancer in a subject, administering an effective amount of at least one probe to the subject, thereby contacting a tissue of the subject with the probe such that the probe binds to the tissue, wherein the at least one probe comprises at least one boron cluster; detecting the probe bound to the tissue, wherein the presence of the probe bound to the tissue is indicative of the cancer in the subject; and delivering the boron cluster to the tissue thereby reducing the severity of and/or slowing the progression of the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Detecting a Tumor

In various embodiments, the present invention provides a method for detecting a tumor in a subject, comprising: administering an effective amount of at least one nanoparticle to the subject, thereby contacting a tumor present in the subject with the at least one nanoparticle such that the at least one nanoparticle binds to the tumor; and detecting the at least one nanoparticle bound to the tumor, wherein the presence of the at least one nanoparticle bound to the tumor is indicative of the presence of the tumor in the subject. In some embodiments, the at least one nanoparticle is a nanoparticle of the present invention.

In various embodiments, the present invention provides a method for detecting a tumor in a subject, comprising: administering an effective amount of at least one probe to the subject, thereby contacting a tumor present in the subject with the at least one probe such that the at least one probe binds to the tumor; and detecting the at least one probe bound to the tumor, wherein the presence of the at least one probe bound to the tumor is indicative of the presence of the tumor in the subject. In some embodiments, the at least one probe is a probe of the present invention.

Methods for Detecting a Tumor Margin of a Tumor

In various embodiments the present invention provides a method for detecting a tumor margin in a subject, comprising: administering an effective amount of at least one nanoparticle to the subject, thereby contacting a tumor present in the subject with the at least one nanoparticle such that the at least one nanoparticle binds to the tumor; and detecting the at least one nanoparticle bound to the tumor, wherein the presence of the at least one nanoparticle bound to the tumor is indicative of the tumor margin of the tumor in the subject. In some embodiments, the at least one nanoparticle is a nanoparticle of the present invention. In some embodiments, the at least one nanoparticle is detected using magnetic resonance imaging. In some embodiments, the at least one nanoparticle is detected using fluorescence imaging. In some embodiments, the at least one nanoparticle is detected using magnetic resonance imaging and fluorescence imaging. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof. In some embodiments, the method further comprises detecting and/or identifying the tumor margin before surgery. In some embodiments, the method further comprises detecting and/or identifying the tumor margin during surgery.

In various embodiments the present invention provides a method for detecting a tumor margin in a subject, comprising: administering an effective amount of at least one probe to the subject, thereby contacting a tumor present in the subject with the at least one probe such that the at least one probe binds to the tumor; and detecting the at least one probe bound to the tumor, wherein the presence of the at least one probe bound to the tumor is indicative of the tumor margin of the tumor in the subject. In some embodiments, the at least one probe is a probe of the present invention. In some embodiments, the at least one probe is detected using magnetic resonance imaging. In some embodiments, the at least one probe is detected using fluorescence imaging. In some embodiments, the at least one probe is detected using magnetic resonance imaging and fluorescence imaging. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof. In some embodiments, the method further comprises detecting and/or identifying the tumor margin before surgery. In some embodiments, the method further comprises detecting and/or identifying the tumor margin during surgery.

Treatments/Therapies and Additional Treatments/Therapies

In some embodiments, the method further comprises treating the subject with a therapy or treatment and/or administering a therapy or treatment to the subject and/or selecting a therapy or treatment for the subject and/or providing a therapy or treatment to the subject. In some embodiments, the treatment is a treatment for cancer. In some embodiments, the treatment is a cancer treatment. In some embodiments, the therapy is a therapy for cancer. In some embodiments, the therapy is a cancer therapy.

In some embodiments, the methods of the present invention may optionally further comprise simultaneously or sequentially administering a therapy or treatment to the subject. Non-limiting examples of treatments and therapies include pharmacological therapy, biological therapy, cell therapy, gene therapy, chemotherapy, radiation therapy, hormonal therapy, surgery, immunotherapy, or combinations thereof.

In some embodiments, the method further comprises treating the subject with an additional therapy or treatment and/or administering an additional therapy or treatment to the subject and/or selecting an additional therapy or treatment for the subject and/or providing an additional therapy or treatment to the subject. In some embodiments, the additional treatment is a treatment for cancer. In some embodiments, the additional treatment is a cancer treatment. In some embodiments, the additional therapy is a therapy for cancer. In some embodiments, the additional therapy is a cancer therapy.

In some embodiments, the methods of the present invention may optionally further comprise simultaneously or sequentially administering an additional therapy or treatment to the subject. Non-limiting examples of additional treatments and therapies include pharmacological therapy, biological therapy, cell therapy, gene therapy, chemotherapy, radiation therapy, hormonal therapy, surgery, immunotherapy, or combinations thereof.

In some embodiments, chemotherapy may comprise the use of chemotherapeutic agents. In some embodiments, chemotherapeutic agents may be selected from any one or more of cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or tele-therapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, immunotherapy may comprise, for example, use of cancer vaccines and/or sensitized antigen presenting cells. In some embodiments, therapies include targeting cells in the tumor microenvironment or targeting immune cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, hormonal therapy can include, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

Some embodiments of the present invention can be defined as any of the following numbered paragraphs:

95. A nanoparticle, comprising:
a core, wherein the core comprises at least one iron oxide;
a shell surrounding the core, wherein the shell comprises at least one polymer;
at least one boron cluster; and
at least one targeting moiety attached to the shell.
96. The nanoparticle of paragraph 95, wherein the at least one iron oxide is selected from the group consisting of FeO, Fe₂O₃, and combinations thereof.
97. The nanoparticle of paragraph 95, wherein the at least one polymer is at least one biocompatible polymer.
98. The nanoparticle of paragraph 95, wherein the at least one polymer is at least one polysaccharide.
99. The nanoparticle of paragraph 95, wherein the at least one polymer is selected from the group consisting of at least one dextran, at least one unfunctionalized dextran, at least one functionalized dextran, at least one unsubstituted dextran, at least one substituted dextran, and combinations thereof.
100. The nanoparticle of paragraph 95, wherein the at least one polymer is selected from the group consisting of carboxymethyl dextran, at least one dextran, and combinations thereof.
101. The nanoparticle of paragraph 99 or paragraph 100, wherein the at least one dextran is selected from the group consisting of a class 1 dextran, a class 2 dextran, a class 3 dextran, and combinations thereof.
102. The nanoparticle of paragraph 95, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
103. The nanoparticle of paragraph 95, further comprising at least one drug.
104. The nanoparticle of paragraph 103, wherein the drug is encapsulated in the nanoparticle.
105. The nanoparticle of paragraph 103, wherein the at least one drug is a therapeutic agent.
106. The nanoparticle of paragraph 103, wherein the at least one drug is selected from the group consisting of docetaxel (DXT), paclitaxel (PXT), bortezomib (Bort), cobozentanib (cabo), brefeldin A (BFA), and combinations thereof.
107. The nanoparticle of paragraph 95, further comprising at least one fluorescent dye.
108. The nanoparticle of paragraph 107, wherein the at least one fluorescent dye is encapsulated in the nanoparticle.
109. The nanoparticle of paragraph 107 or 108, wherein the nanoparticle is selected from angiopep-FH(DiR) and angiopep-FH(HMC).
110. The nanoparticle of paragraph 107, wherein the at least one fluorescent dye is a near infrared fluorescent dye.
111. The nanoparticle of paragraph 107, wherein the at least one fluorescent dye is selected from the group consisting of DiI, DiR, heptamethine cyanine (HMC), IR820, and combinations thereof.
112. The nanoparticle of paragraph 107, wherein the nanoparticle is a multimodal nanoparticle.
113. The nanoparticle of paragraph 103, further comprising at least one fluorescent dye.
114. The nanoparticle of paragraph 113, wherein the at least one fluorescent dye is encapsulated in the nanoparticle.
115. The nanoparticle of paragraph 113, wherein the at least one fluorescent dye is a near infrared fluorescent dye.
116. The nanoparticle of paragraph 113, wherein the at least one fluorescent dye is selected from the group consisting of DiI, DiR, heptamethine cyanine (HMC), IR820, and combinations thereof.
117. The nanoparticle of paragraph 113, wherein the nanoparticle is a multimodal nanoparticle.
118. A method for detecting and treating a cancer in a subject, comprising:
administering an effective amount of at least one nanoparticle of paragraph 103 or paragraph 113 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one boron cluster to the tissue thereby treating the cancer in the subject.
119. A method for detecting a cancer in a subject, comprising:
administering an effective amount of at least one nanoparticle of paragraph 95 or paragraph 113 to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.
120. The method of paragraph 119, further comprising administering a treatment to the subject.
121. A method for diagnosing and treating a cancer in a subject, comprising:
administering an effective amount of at least one nanoparticle of paragraph 103 or paragraph 113 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one boron cluster to the tissue thereby treating the cancer in the subject.
122. A method for diagnosing a cancer in a subject, comprising:
administering an effective amount of at least one nanoparticle of paragraph 95 or paragraph 113 to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.
123. The method of paragraph 122, further comprising administering a treatment to the subject.
124. A method for treating a cancer in a subject, comprising:
administering an effective amount of at least one nanoparticle of paragraph 103 or paragraph 113 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one boron cluster to the tissue thereby treating the cancer in the subject.
125. The method of any one of paragraphs 118, 119, 121, 122, or 124, wherein the at least one nanoparticle is detected using magnetic resonance imaging.
126. The method of any one of paragraphs 118, 119, 121, 122, or 124, wherein the at least one nanoparticle is detected using fluorescence imaging.
127. The method of paragraph 126, wherein the fluorescence imaging is selected from the group consisting of near infrared fluorescence imaging, intraoperative fluorescence imaging, and combinations thereof.
128. The method of any one of paragraphs 118, 119, 121, 122, or 124, wherein the at least one nanoparticle is detected using magnetic resonance imaging and fluorescence imaging.
129. The method of paragraph 128, wherein the fluorescence imaging is selected from the group consisting of near infrared fluorescence imaging, intraoperative fluorescence imaging, and combinations thereof.
130. The method of any one of paragraphs 118, 119, 121, 122, or 124, wherein the cancer is selected from the group consisting of lung cancer, breast cancer, ovarian cancer, pancreatic cancer, head cancer, neck cancer, skin cancer, prostate cancer, brain cancer, and combinations thereof.
131. The method of paragraph 118, 119, 121, 122, or 124, wherein the cancer is metastasized.
132. The method of any one of paragraphs 118, 119, 121, 122, or 124, wherein the tissue is selected from the group consisting of cancerous tissue, cancer tissue, tumor, tumor tissue, and combinations thereof.
133. The method of any one of paragraphs 118, 121, or 124, further comprising administering at least one additional therapy to the subject.
134. The method of paragraph 133, wherein the additional therapy is selected from the group consisting of pharmacological therapy, biological therapy, cell therapy, gene therapy, chemotherapy, radiation therapy, hormonal therapy, surgery, immunotherapy, and combinations thereof.
135. A probe comprising at least one coated iron oxide nanoparticle; at least one boron cluster; and at least one targeting moiety.
136. The probe of paragraph 135, wherein the at least one targeting moiety is attached to the at least one coated iron oxide nanoparticle.
137. The probe of paragraph 135, wherein the at least one coated iron oxide nanoparticle is selected from the group consisting of Ferumoxytol, Ferumoxides, Ferucarbotran, Ferumoxtran-10, NC100150, VSOP C184, and combinations thereof.
138. The probe of paragraph 135, further comprising at least one drug.
139. The probe of paragraph 138, wherein the at least one drug is encapsulated in the probe.
140. The probe of paragraph 138, wherein the at least one drug is a therapeutic agent.
141. The probe of paragraph 138, wherein the at least one drug is selected from the group consisting of docetaxel (DXT), paclitaxel (PXT), bortezomib (Bort), cobozentanib (cabo), brefeldin A (BFA), and combinations thereof.
142. The probe of paragraph 135, further comprising at least one fluorescent dye.
143. The probe of paragraph 142, wherein the at least one fluorescent dye is encapsulated in the probe.
144. The probe of paragraph 142 or 143, wherein the probe is selected from angiopep-FH(DiR) and angiopep-FH(HMC).
145. The probe of paragraph 142, wherein the at least one fluorescent dye is a near infrared fluorescent dye.
146. The probe of paragraph 142, wherein the at least one fluorescent dye is selected from the group consisting of DiI, DiR, heptamethine cyanine (HMC), IR820, and combinations thereof.
147. The probe of paragraph 142, wherein the probe is a multimodal probe.
148. The probe of paragraph 138, further comprising at least one fluorescent dye.
149. The probe of paragraph 148, wherein the at least one fluorescent dye is encapsulated in the probe.
150. The probe of paragraph 148, wherein the at least one fluorescent dye is a near infrared fluorescent dye.
151. The probe of paragraph 148, wherein the at least one fluorescent dye is selected from the group consisting of DiI, DiR, heptamethine cyanine (HMC), IR820, and combinations thereof
152. The probe of paragraph 148, wherein the probe is a multimodal probe.
153. A method for detecting and treating a cancer in a subject, comprising:
administering an effective amount of at least one probe of paragraph 138 or paragraph 148 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the at least one boron cluster to the tissue thereby treating the cancer in the subject.
154. A method for detecting a cancer in a subject, comprising:
administering an effective amount of at least one probe of paragraph 135 or paragraph 148 to the subject, thereby contacting a tissue of the subject with the at least one probe such that the at least one probe binds to the tissue; and
detecting the at least one probe bound to the tissue, wherein the presence of the at least one probe bound to the tissue is indicative of the cancer in the subject.
155. The method of paragraph 154, further comprising administering a treatment to the subject.
156. A method for diagnosing and treating a cancer in a subject, comprising:
administering an effective amount of at least one probe of paragraph 138 or paragraph 148 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the at least one boron cluster to the tissue thereby treating the cancer in the subject.
157. A method for diagnosing a cancer in a subject, comprising:
administering an effective amount of at least one probe of paragraph 135 or paragraph 148 to the subject, thereby contacting a tissue of the subject with the at least one probe such that the at least one probe binds to the tissue; and
detecting the at least one probe bound to the tissue, wherein the presence of the at least one probe bound to the tissue is indicative of the cancer in the subject.
158. The method of paragraph 157, further comprising administering a treatment to the subject.
159. A method for treating a cancer in a subject, comprising:
administering an effective amount of at least one probe of paragraph 138 or paragraph 148 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the boron cluster to the tissue thereby treating the cancer in the subject.
160. The method of any one of paragraphs 153, 154, 156, 157, or 159, wherein the at least one probe is detected using magnetic resonance imaging.
161. The method of any one of paragraphs 153, 154, 156, 157, or 159, wherein the at least one probe is detected using fluorescence imaging.
162. The method of paragraph 161, wherein the fluorescence imaging is selected from the group consisting of near infrared fluorescence imaging, intraoperative fluorescence imaging, and combinations thereof.
163. The method of any one of paragraphs 153, 154, 156, 157, or 159, wherein the at least one probe is detected using magnetic resonance imaging and fluorescence imaging.
164. The method of paragraph 163, wherein the fluorescence imaging is selected from the group consisting of near infrared fluorescence imaging, intraoperative fluorescence imaging, and combinations thereof.
165. The method of any one of paragraphs 153, 154, 156, 157, or 159, wherein the cancer is selected from the group consisting of lung cancer, breast cancer, ovarian cancer, pancreatic cancer, head cancer, neck cancer, skin cancer, prostate cancer, brain cancer, and combinations thereof.
166. The method of any one of paragraphs 153, 154, 156, 157, or 159, wherein the cancer is metastasized.
167. The method of any one of paragraphs 153, 154, 156, 157, or 159, wherein the tissue is selected from the group consisting of cancerous tissue, cancer tissue, tumor, tumor tissue, and combinations thereof.
168. The method of any one of paragraphs 153, 156, or 159, further comprising administering at least one additional therapy to the subject.
169. The method of paragraph 168, wherein the additional therapy is selected from the group consisting of pharmacological therapy, biological therapy, cell therapy, gene therapy, chemotherapy, radiation therapy, hormonal therapy, surgery, immunotherapy, and combinations thereof.
170. A pharmaceutical composition comprising at least one nanoparticle of any one of paragraphs 95 to 117.
171. A pharmaceutical composition comprising at least one probe of any one of paragraphs 135 to 152.
172. The nanoparticle of paragraph 95, wherein the nanoparticle further comprises at least one boron cluster.
173. The probe of paragraph 135, wherein the probe further comprises at least one boron cluster.
174. The nanoparticle of paragraph 172, wherein the targeting moiety targets glioma.
175. The nanoparticle of paragraph 174, wherein the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), and combinations thereof.
176. The nanoparticle of paragraph 103, wherein the targeting moiety targets glioma.
177. The nanoparticle of paragraph 176, wherein the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), and combinations thereof.
178. The probe of paragraph 173, wherein the targeting moiety targets glioma.
179. The probe of paragraph 178, wherein the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), and combinations thereof.
180. The probe of paragraph 138, wherein the targeting moiety targets glioma.
181. The probe of paragraph 180, wherein the targeting moiety is selected from the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), and combinations thereof.

Some embodiments of the present invention can be defined as any of the following numbered paragraphs:

182. A nanovehicle, comprising:

a core, wherein the core comprises at least one iron oxide;

a shell surrounding the core, wherein the shell comprises at least one polymer; and

at least one boron cluster.

183. The nanovehicle of paragraph 182, wherein the at least one iron oxide is selected from the group consisting of FeO, Fe₂O₃, and a combination thereof.
184. The nanovehicle of paragraph 182, wherein the at least one polymer is at least one biocompatible polymer.
185. The nanovehicle of paragraph 182, wherein the at least one polymer is at least one polysaccharide.
186. The nanovehicle of paragraph 182, wherein the at least one polymer is one selected from the group consisting of at least one dextran, at least one unfunctionalized dextran, at least one functionalized dextran, at least one unsubstituted dextran, at least one substituted dextran, and combinations thereof.
187. The nanovehicle of paragraph 182, wherein the at least one polymer is selected from the group consisting of carboxymethyl dextran, at least one dextran, and combinations thereof.
188. The nanovehicle of paragraph 186 or paragraph 187, wherein the at least one dextran is selected from the group consisting of a class 1 dextran, a class 2 dextran, a class 3 dextran, and combinations thereof.
189. The nanovehicle of paragraph 182, wherein the at least one boron cluster is selected from the group consisting of unfunctionalized boron cluster, functionalized boron cluster, and combinations thereof.
190. The nanovehicle of paragraph 182, wherein the at least one boron cluster is selected from the group consisting of an unfunctionalized B₁₂ boron cluster, functionalized B₁₂ boron cluster, and combinations thereof.
191. The nanovehicle of paragraph 182, further comprising at least one targeting ligand attached to the shell.
192. The nanovehicle of paragraph 182 or paragraph 191, further comprising at least one drug.
193. The nanovehicle of paragraph 182, paragraph 191, or paragraph 192, further comprising at least one fluorescent dye.
194. A method of treating, reducing the severity of and/or slowing the progression of a disease, disorder, or disease condition in a subject, comprising:
providing at least one nanovehicle, wherein the at least one nanovehicle comprises: a core, wherein the core comprises at least one iron oxide; a shell surrounding the core, wherein the shell comprises at least one polymer; and at least one boron cluster;
administering a therapeutically effective amount of the at least one nanovehicle to the subject; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.
195. A nanoparticle, comprising:

a core, wherein the core comprises at least one iron oxide;

a shell surrounding the core, wherein the shell comprises at least one polymer;

at least one boron cluster; and

at least one targeting moiety attached to the shell.

196. The nanoparticle of paragraph 195, wherein the at least one iron oxide is selected from the group consisting of FeO, Fe₂O₃, and combinations thereof.
197. The nanoparticle of paragraph 195, wherein the at least one polymer is at least one biocompatible polymer.
198. The nanoparticle of paragraph 195, wherein the at least one polymer is at least one polysaccharide.
199. The nanoparticle of paragraph 195, wherein the at least one polymer is selected from the group consisting of at least one dextran, at least one unfunctionalized dextran, at least one functionalized dextran, at least one unsubstituted dextran, at least one substituted dextran, and combinations thereof.
200. The nanoparticle of paragraph 195, wherein the at least one polymer is selected from the group consisting of carboxymethyl dextran, at least one dextran, and combinations thereof.
201. The nanoparticle of paragraph 199 or paragraph 200, wherein the at least one dextran is selected from the group consisting of a class 1 dextran, a class 2 dextran, a class 3 dextran, and combinations thereof.
202. The nanoparticle of paragraph 195, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
203. The nanoparticle of paragraph 195, further comprising at least one drug.
204. The nanoparticle of paragraph 195, further comprising at least one fluorescent dye.
205. The nanoparticle of paragraph 203, further comprising at least one fluorescent dye.
206. A method for detecting a cancer in a subject, comprising:

administering an effective amount of at least one nanoparticle of claim 195 or claim 203 to the subject, thereby contacting a tissue of the subject with the at least one nanoparticle such that the at least one nanoparticle binds to the tissue; and

detecting the at least one nanoparticle bound to the tissue, wherein the presence of the at least one nanoparticle bound to the tissue is indicative of the cancer in the subject.

207. The method of paragraph 206, further comprising administering a treatment to the subject.
208. A probe comprising at least one coated iron oxide nanoparticle; and at least one targeting moiety.
209. The probe of paragraph 208, wherein the at least one targeting moiety is attached to the at least one coated iron oxide nanoparticle.
210. The probe of paragraph 208, wherein the at least one coated iron oxide nanoparticle is selected from the group consisting of Ferumoxytol, Ferumoxides, Ferucarbotran, Ferumoxtran-10, NC100150, VSOP C184, and combinations thereof.
211. The probe of paragraph 208, further comprising at least one drug.
212. The probe of paragraph 208, further comprising at least one fluorescent dye.
213. The probe of paragraph 211, further comprising at least one fluorescent dye.
214. A method for detecting a cancer in a subject, comprising:

administering an effective amount of at least one probe of claim 208 or claim 213 to the subject, thereby contacting a tissue of the subject with the at least one probe such that the at least one probe binds to the tissue; and

detecting the at least one probe bound to the tissue, wherein the presence of the at least one probe bound to the tissue is indicative of the cancer in the subject.

215. The method of paragraph 214, further comprising administering a treatment to the subject.
216. The method of paragraph 194, wherein the disease is a cancer.
217. The nanovehicle of paragraph 182, wherein the nanovehicle further comprises at least one targeting moiety.
218. The method of paragraph 194, wherein the nanovehicle further comprises at least one targeting moiety.
219. The method of paragraph 207 or claim 215, wherein the treatment is a cancer treatment.
220. The method of paragraph 206, wherein the nanoparticle is detected by an imaging method.
221. The method of paragraph 214, wherein the probe is detected by an imaging method.
222. The method of paragraph 220 or paragraph 221, wherein the imaging method is selected from the group consisting of magnetic resonance imaging, fluorescence imaging, and combinations thereof.
223. The nanovehicle of paragraph 182, wherein the nanovehicle is a nanoparticle.
224. The method of paragraph 206 or paragraph 214, wherein the tissue is selected from the group consisting of cancerous tissue, cancer tissue, tumor, tumor tissue, and combinations thereof.
225. A method of treating, reducing the severity of and/or slowing the progression of cancer in a subject, comprising:
providing at least one nanovehicle, wherein the at least one nanovehicle comprises: a core, wherein the core comprises at least one iron oxide; a shell surrounding the core, wherein the shell comprises at least one polymer; at least one boron cluster; and at least one targeting ligand attached to the shell;
administering a therapeutically effective amount of the at least one nanovehicle to the subject, thereby contacting a tissue of the subject with the at least one nanovehicle,
wherein the tissue is selected from the group consisting of cancerous tissue, non-cancerous tissue, and combinations thereof, and
wherein the nanovehicle selectively binds to the cancerous tissue; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the cancer in the subject.
226. The method of paragraph 225, further comprising at least one drug.
227. The method of paragraph 225 or paragraph 226, further comprising at least one fluorescent dye.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention. The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention

### Example 1

Boron Cluster Encapsulation into Feraheme.

Various synthesized boron clusters (BC) are encapsulated into Feraheme (FH) and these various preparations are denoted as FH(BC) (e.g., FH(BC) nanoparticles).

The various FH(BC) preparations, particularly those with the highest loading of boron clusters (BC), are selected for further study. The stability of the FH(BC) preparations, as well as the stability of the encapsulation of the boron cluster (BC) is examined in order to identify those FH(BC) preparations that achieve the highest loading without compromising nanoparticle stability. The stability of the FH(BC) preparations in water, phosphate buffer saline and various biological medium, including serum are studied. FH(BC) preparations that stably encapsulate the boron clusters (BC) without premature release are selected for further study.

Studies to investigate the toxicity of various FH(BC) formulations in a panel of healthy and cancer cells are conducted. Studies are conducted using FH(BC) nanoparticles exposed to cell cultures and tissue samples using a neutron source. These studies address whether boron-containing particles (e.g., FH(BC) nanoparticles) can elicit tissue damage compared to the controls that would be exposed to Feraheme particles without boron-containing payload.

### Example 2

Biological Studies: Uptake and Toxicity Studies in Cultured Cells and Animal Models.

One purpose of this example is to study the tumor accumulation of the FH(BC) preparations (e.g., FH(BC) nanoparticles) upon in vivo administration in mouse models. Of particular importance is the study of the presence of boron in the brain tumor versus the healthy brain. Ideally, a significant amount of boron is observed in the tumor with little to no boron present in the healthy brain.

FH(BC) preparations (e.g., FH(BC) nanoparticles), particularly the most optimal and blood serum stable FH(BC) preparations, will be injected in GBM-bearing mice. Glioblastoma multiforme (GBM) is an aggressive form of brain cancer that is without an efficient treatment and has a poor prognosis.

The mice will be then sacrificed at different time points (e.g., 3, 6, 12, 24, 48, 72 hours) and their brains and tumors taken out as well as other organs such as liver, kidney, heart, and the amount of boron in each will be determined by inductive couple plasma mass spectrometry (ICP-MS). As boron is not an element present in human tissue, the presence of boron in the brain tumor or any other organ will indicate the presence of the boron clusters.

These experiments are repeated several times to obtain statistical significant results. In addition, animal toxicity, and bio distribution studies are performed and compared to those observed with Feraheme with no boron.

FH(BC) preparations (e.g., FH(BC) nanoparticles) that deliver a large amount of boron to the GBM tumors with minimal to no accumulation in the healthy brain are identified.

### Example 3

Selective Targeting and Crossing of the Blood Brain Barrier.

We modify the surface of FH(BC) preparations (e.g., FH(BC) nanoparticles) with targeting ligands (TL) known to cross the blood brain barrier and accumulate in GBM tumors. The resulting TL-FH(BC) preparations (e.g., TL-FH(BC) nanoparticles) are studied in animal models as described above in Experiment 2 and the results are compared with those obtained with FH(BC) from Experiment 2.

These experiments are repeated several times to obtain statistical significant results. In addition, animal toxicity, and bio distribution studies are performed and compared to those observed with TL-Feraheme with no boron.

TL-FH(BC) preparations (e.g., TL-FH(BC) nanoparticles) that deliver a large amount of boron to the GBM tumors with minimal to no accumulation in the healthy brain are identified.

### Example 4

Biological Studies: Treatment of Animals with a Neutron Source.

Animals are treated with FH(BC) or TL-FH(BC) and are exposed to a neutron beam (test) or not exposed to a neutron beam (control). The reduction in tumor volume at different time points after neutron beam treatment is visualized by magnetic resonance imaging (MRI) or by necroscopy (animal autopsy). Histology and molecular profiling of the tumors are examined and compared to those of neighboring tissues.

### Example 5

Multimodal HMC-FH nanoconjugates are sensitive dual near infrared and magnetic probes. Considering its exquisite tumor affinity and desirable NIRF properties, HMC was conjugated to FH for the fluorescent intraoperative detection of prostate cancer tumor. To achieve this, we have initially modified HMC with a lysine linker to yield HMC-Lys (FIG. 47); that is then conjugated onto the carboxylic acid groups in FH's carboxymethyl dextran coating. HMC conjugation does not affect the size, polydispersity and stability of the nanoparticles in aqueous buffers.

### Example 6

PSMA-Targeting-Feraheme nanoparticles. In this set of experiments, glutamate was conjugated to the carboxylic acid groups on Feraheme. The amine group (—NH₂) on the glutamate was conjugated with the carboxylic acid group (—COOH) on the carboxymethyldextran coated of Feraheme using EDC/HNS chemistry. This results in conjugation of multiple glutamate ligands to the surface of Feraheme. The resulting Glutamate-Feraheme (GLU-FH) nanoparticles are characterized by DLS (size), and zeta potential (charge). Both the Glutamate conjugate (GLU-FH) and the folate conjugate (FOL-FH) have been synthesized.

### Example 7

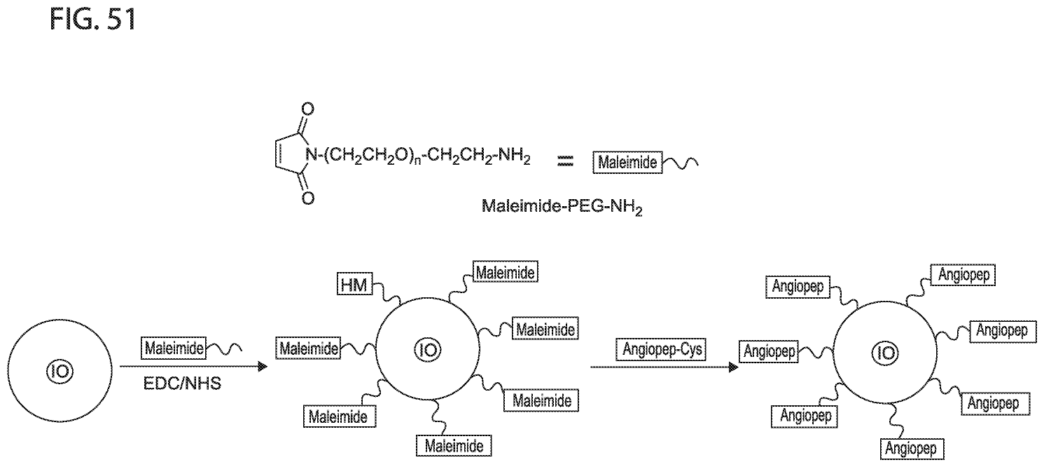

The Angiopep peptide was custom-ordered with a cysteine residue on the carboxylic acid end (TFFYGGSRGKRNNFKTEEYC) (SEQ ID NO: 1) to facilitate binding to Feraheme via a maleimide linker. To achieve this, the carboxylic acid groups on Feraheme were first conjugated with a Maleimide-PEG-Amine linker using EDC/NHS ester chemistry and the resulting Maleimide-PEG-Feraheme was then reacted with the cysteine modified Angiopep (FIG. 51). The cysteine's sulfhydryl group on Angiopep exclusively reacts with the maleimide double bond forming a stable linker that conjugates Angiopep to the surface of Feraheme. The resulting Angiopep-Feraheme nanoparticles are characterized by DLS (size), and zeta potential (charge).

### Example 8

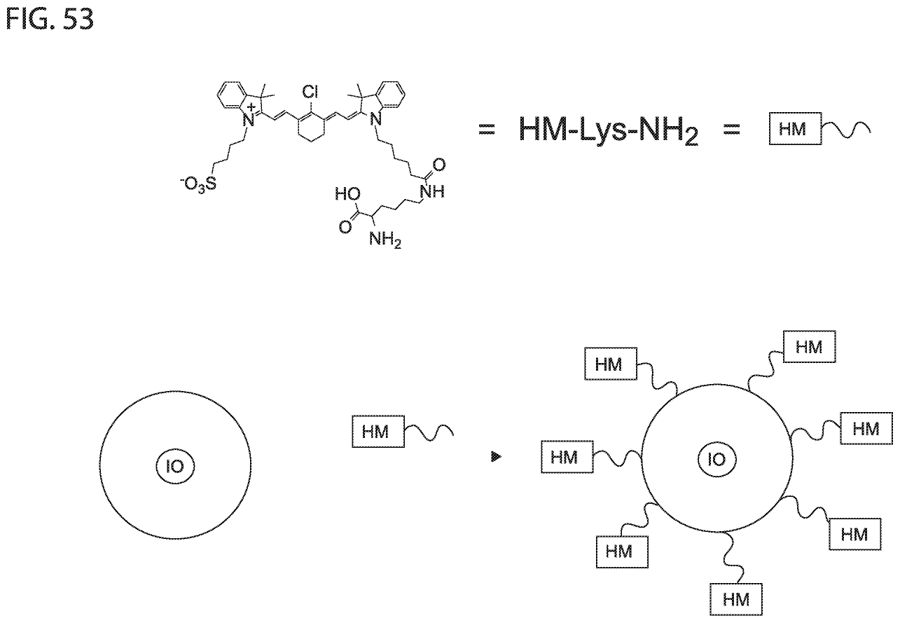

Multimodal HM-Feraheme Nanoparticle. A heptamethine-lysine conjugate (HM-Lys-NH₂) was synthesized. The amine group (—NH₂) on the lysine amino acid group was conjugated with the carboxylic acid group (—COOH) on the carboxymethyldextran coated of Feraheme using EDC/HNS chemistry. This resulted in conjugation of multiple heptamethine dyes to the surface of Feraheme via a lysine flexible linker (FIG. 53). The resulting HM-Feraheme nanoparticles are characterized by DLS (size), zeta potential (charge), and fluorescence spectroscopy. These nanoparticle-conjugates are stable, highly fluorescent and no loss of their magnetic properties is expected.

In various embodiments, the present invention provides for the pre-operative identification of tumor margins by magnetic resonance imaging, and during surgery using fluorescence imaging guided surgery. In various embodiments, the present invention provides for the tumor-targeted delivery of drugs and boron clusters using an iron oxide (e.g., Feraheme) formulation comprising at least one boron cluster that targets OATP receptors in cancer cells and visualization of drug and boron cluster delivery by magnetic resonance imaging (MRI) or fluorescence imaging. In some embodiments, the fluorescence imaging is selected from the group consisting of near infrared fluorescence imaging, intraoperative fluorescence imaging, and combinations thereof.

In some embodiments, the present invention can be offered to cancer patients undergoing chemotherapy. For example, Feraheme is currently administered in the clinic for the treatment of anemia at a dose of 510 mg, followed by a second administration within 3-8 days. Without being bound by theory, for imaging and drug and boron cluster delivery purposes, a lower amount may be able to be used. In some embodiments, during chemotherapy, a once or twice a month administration of the nanoparticles, probes, or pharmaceutical composition thereof may be used. In some embodiments, for diagnostics and the assessment of tumor margins before and during surgery a one-time dose may be used.

### REFERENCES

1. Verma, S., et al., The Current State of MR Imaging-targeted Biopsy Techniques for Detection of Prostate Cancer. Radiology, 2017. 285(2): p. 343-356.
2. Acar, C., et al., Advances in sentinel node dissection in prostate cancer from a technical perspective. Int J Urol, 2015. 22(10): p. 898-909.
3. Brouwer, O R., et al., Image navigation as a means to expand the boundaries of fluorescence-guided surgery. Phys Med Biol, 2012. 57(10): p. 3123-36.
4. Cornejo-Davila, V., et al., Use of near infrared fluorescence during robot-assisted laparoscopic partial nephrectomy. Actas Urol Esp, 2016. 40(3): p. 190-4.
5. Greco, F., et al., Current perspectives in the use of molecular imaging to target surgical treatments for genitourinary cancers. Eur Urol, 2014. 65(5): p. 947-64.
6. Hall, M. A., et al., Imaging prostate cancer lymph node metastases with a multimodality contrast agent. Prostate, 2012. 72(2): p. 129-46.
7. Liss, M. A., et al., Robotic-assisted fluorescence sentinel lymph node mapping using multimodal image guidance in an animal model. Urology, 2014. 84(4): p. 982 e9-14.
8. Lutje, S., et al., Dual-Modality Image-Guided Surgery of Prostate Cancer with a Radiolabeled Fluorescent Anti-PSMA Monoclonal Antibody. J Nucl Med, 2014. 55(6): p. 995-1001.
9. Sonn, G. A., et al., Fluorescent Image-Guided Surgery with an Anti-Prostate Stem Cell Antigen (PSCA) Diabody Enables Targeted Resection of Mouse Prostate Cancer Xenografts in Real Time. Clin Cancer Res, 2016. 22(6): p. 1403-12.
10. Wang, X., et al., Development of targeted near-infrared imaging agents for prostate cancer. Mol Cancer Ther, 2014. 13(11): p. 2595-606.
11. Xia, L., et al., Near-infrared Intraoperative Molecular Imaging Can Identify Metastatic Lymph Nodes in Prostate Cancer. Urology, 2017. 106: p. 133-138.
12. Yuen, K., et al., Intraoperative Fluorescence Imaging for Detection of Sentinel Lymph Nodes and Lymphatic Vessels during Open Prostatectomy using Indocyanine Green. J Urol, 2015. 194(2): p. 371-7.
13. Litwin, M. S. and H. J. Tan, The Diagnosis and Treatment of Prostate Cancer: A Review. JAMA, 2017. 317(24): p. 2532-2542.
14. Auerbach, M., et al., Safety and efficacy of total dose infusion of 1,020 mg of ferumoxytol administered over 15 min. Am J Hematol, 2013. 88(11): p. 944-7.
15. Cheng, K., et al., Magnetic antibody-linked nanomatchmakers for therapeutic cell targeting. Nat Commun, 2014. 5: p. 4880.
16. Lu, M., et al., FDA report: Ferumoxytol for intravenous iron therapy in adult patients with chronic kidney disease. Am J Hematol, 2010. 85(5): p. 315-9.
17. Bashir, M. R., et al., Emerging applications for ferumoxytol as a contrast agent in MRI. J Magn Reson Imaging, 2015. 41(4): p. 884-98.
18. Hope, M. D., et al., Vascular Imaging With Ferumoxytol as a Contrast Agent. AIR Am J Roentgenol, 2015. 205(3): p. W366-73.
19. Toth, G. B., et al., Current and potential imaging applications of ferumoxytol for magnetic resonance imaging. Kidney Int, 2017. 92(1): p. 47-66.
20. Schwenk, M. H., Ferumoxytol: a new intravenous iron preparation for the treatment of iron deficiency anemia in patients with chronic kidney disease. Pharmacotherapy, 2010. 30(1): p. 70-9.
21. Santra, S., et al., Drug/dye-loaded, multifunctional iron oxide nanoparticles for combined targeted cancer therapy and dual optical/magnetic resonance imaging. Small, 2009. 5(16): p. 1862-8.
22. Kaittanis, C., et al., Environment-responsive nanophores for therapy and treatment monitoring via molecular MRJ quenching. Nat Commun, 2014. 5: p. 3384.
23. Li, R., et al., Be Active or Not: the Relative Contribution of Active and Passive Tumor Targeting of Nanomaterials. Nanotheranostics, 2017. 1(4): p. 346-357.
24. Miller, M. A., S. Arlauckas, and R. Weissleder, Prediction of Anti-cancer Nanotherapy Efficacy by Imaging. Nanotheranostics, 2017. 1(3): p. 296-312.
25. Obaidat, A., M. Roth, and B. Hagenbuch, The expression and function of organic anion transporting polypeptides in normal tissues and in cancer. Annu Rev Pharmacol Toxicol, 2012. 52: p. 135-51.
26. Shi, C., J. B. Wu, and D. Pan, Review on near-infrared heptamethine cyanine dyes as theranostic agents for tumor imaging, targeting, and photodynamic therapy. J Biomed Opt, 2016. 21(5): p. 50901.
27. Roth, M., A. Obaidat, and B. Hagenbuch, OATPs, OA Ts and OCTs: the organic anion and cation transporters of the SLCO and SLC22A gene superfamilies. Br J Pharmacol, 2012. 165(5): p. 1260-87.
28. Buxhofer-Ausch, V., et al., Tumor-specific expression of organic anion-transporting polypeptides: transporters as novel targets for cancer therapy. J Drug Deliv, 2013. 2013: p. 863539.
29. Pressler, H., et al., Expression of OATP family members in hormone-related cancers: potential markers of progression. PLoS One, 2011. 6(5): p. e20372.
30. Yuan, J., et al., Nearinfrared fluorescence imaging of prostate cancer using heptamethine carbocyanine dyes. Mol Med Rep, 2015. 11(2): p. 821-8.
31. Shi, C., et al., Heptamethine carbocyanine dye-mediated near-infrared imaging of canine and human cancers through the HIF-1alpha/OATPs signaling axis. Oncotarget, 2014. 5(20): p. 10114-26.
32. Yang, X., et al., Optical imaging of kidney cancer with novel near infrared heptamethine carbocyaninefluorescent dyes. J Urol, 2013. 189(2): p. 702-710.
33. Yang, X., et al., Near IR heptamethine cyanine dye-mediated cancer imaging. Clin Cancer Res, 2010. 16(10): p. 2833-44.
34. Wu, J. B., et al., Near-infrared fluorescence imaging of cancer mediated by tumor hypoxia and HIF1alpha/OATPs signaling axis. Biomaterials, 2014. 35(28): p. 8175-85.
35. Miller, K. D., et al., Cancer treatment and survivorship statistics, 2016. CA Cancer J Clin, 2016. 66(4): p. 271-89.
36. Siegel, R. L., K. D. Miller, and A. Jemal, Cancer Statistics, 2017. CA Cancer J Clin, 2017. 67(1): p. 7-30.
37. Petrylak, D. P., et al., Docetaxel and estramustine compared with mitoxantrone and prednisone for advanced refractory prostate cancer. N Engl J Med, 2004. 351(15): p. 1513-20.
38. Tannock, I. F., et al., Docetaxel plus prednisone or mitoxantrone plus prednisone for advanced prostate cancer. N Engl J Med, 2004. 351(15): p. 1502-12.
39. Krex, D., et al., Long-term survival with glioblastoma multiforme. Brain, 2007. 130(Pt 10): p. 2596-606.
40. Stupp, R., et al., Radiotherapy plus concomitant and adjuvant temozolomide for glioblastoma. N Engl J Med, 2005. 352(10): p. 987-96.
41. Stupp, R., et al., Maintenance Therapy With Tumor-Treating Fields Plus Temozolomide vs Temozolomide Alone for Glioblastoma: A Randomized Clinical Trial. JAMA, 2015. 314(23): p. 2535-43.
42. Bao, S., et al., Glioma stem cells promote radioresistance by preferential activation of the DNA damage response. Nature, 2006. 444(7120): p. 756-60.
43. Rich, J. N. and S. Bao, Chemotherapy and cancer stem cells. Cell Stem Cell, 2007. 1(4): p. 353-5.
44. Rycaj, K. and D. G. Tang, Cancer stem cells and radioresistance. Int J Radiat Biol, 2014. 90(8): p. 615-21.
45. Tamura, K., et al., Expansion of CD133-positive glioma cells in recurrent de novo glioblastomas after radiotherapy and chemotherapy. J Neurosurg, 2013. 119(5): p. 1145-55.
46. Fujiwara, T., et al., Brefeldin A causes disassembly of the Golgi complex and accumulation of secretory proteins in the endoplasmic reticulum. J Biol Chem, 1988. 263(34): p. 18545-52.
47. Lee, S. A., Y. J. Kim, and C. S. Lee, Brefeldin a induces apoptosis by activating the mitochondrial and death receptor pathways and inhibits focal adhesion kinase-mediated cell invasion. Basic Clin Pharmacol Toxicol, 2013. 113(5): p. 329-38.
48. Lippincott-Schwartz, J., et al., Rapid redistribution of Golgi proteins into the ER in cells treated with brefeldin A: evidence for membrane cycling from Golgi to ER. Cell, 1989. 56(5): p. 801-13.
49. Pommepuy, I., et al., Brefeldin A induces apoptosis and cell cycle blockade in glioblastoma cell lines. Oncology, 2003. 64(4): p. 459-67.
50. Cherfils, J. and P. Melancon, On the action of Brefeldin A on Se7-stimulated membrane-recruitment and GDP/GTP exchange of Arf proteins. Biochem Soc Trans, 2005. 33(Pt 4): p. 635-8.
51. Schlienger, S., et al., ADP-ribosylation factor 1 expression regulates epithelial-mesenchymal transition and predicts poor clinical outcome in triple-negative breast cancer. Oncotarget, 2016. 7(13): p. 15811-27.
52. Chen et al., Angiopep-pluronic F127-conjugated superparamagnetic iron oxide nanoparticles as nanotheranostic agents for BBB targeting. J. Mater. Chem. B, 2014. 2: p. 5666-5675

To provide aspects of the present disclosure, embodiments may employ any number of programmable processing devices that execute software or stored instructions. Physical processors and/or machines employed by embodiments of the present disclosure for any processing or evaluation may include one or more networked (Internet, cloud, WAN, LAN, satellite, wired or wireless (RF, cellular, WiFi, Bluetooth, etc.)) or non-networked general purpose computer systems, microprocessors, filed programmable gate arrays (FPGAs), digital signal processors (DSPs), micro-controllers, smart devices (e.g., smart phones), computer tablets, handheld computers, and the like, programmed according to the teachings of the exemplary embodiments. In addition, the devices and subsystems of the exemplary embodiments can be implemented by the preparation of application-specific integrated circuits (ASICs) or by interconnecting an appropriate network of conventional component circuits. Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, database management software, and the like. Computer code devices of the exemplary embodiments can include any suitable interpretable or executable code mechanism, including but not limited to scripts, interpretable programs, dynamic link libraries (DLLs), Java classes and applets, complete executable programs, and the like. Moreover, processing capabilities may be distributed across multiple processors for better performance, reliability, cost, or other benefits.

Common forms of computer-readable media may include, for example, a floppy disk, a flexible disk, a hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read. Such storage media can also be employed to store other types of data, e.g., data organized in a database, for access, processing, and communication by the processing devices.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Various embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

Many variations and alternative elements have been disclosed in embodiments of the present invention. Still further variations and alternate elements will be apparent to one of skill in the art. Among these variations, without limitation, is the selection of steps, pharmaceutical compositions, administration routes and devices, technologies for the inventive methods, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A nanovehicle, comprising:
a core, wherein the core comprises at least one iron oxide;
a shell surrounding the core, wherein the shell comprises at least one polymer; and
at least one boron cluster.

2. The nanovehicle of claim 1, wherein the at least one iron oxide is selected from the group consisting of FeO, Fe2O3, and a combination thereof.

3. The nanovehicle of claim 1, wherein the at least one polymer is at least one biocompatible polymer, or at least one polysaccharide.

4. (canceled)

5. The nanovehicle of claim 1, wherein the at least one polymer is one selected from the group consisting of at least one dextran, at least one unfunctionalized dextran, at least one functionalized dextran, at least one unsubstituted dextran, at least one substituted dextran, and combinations thereof.

6. The nanovehicle of claim 1, wherein the at least one polymer is selected from the group consisting of carboxymethyl dextran, at least one dextran, and combinations thereof.

7. The nanovehicle of claim 5, wherein the at least one dextran is selected from the group consisting of a class 1 dextran, a class 2 dextran, a class 3 dextran, and combinations thereof.

8. The nanovehicle of claim 1, wherein the at least one boron cluster is selected from the group consisting of unfunctionalized boron cluster, functionalized boron cluster, and combinations thereof.

9. The nanovehicle of claim 1, wherein the at least one boron cluster is selected from the group consisting of an unfunctionalized B12 boron cluster, functionalized B12 boron cluster, and combinations thereof.

10. The nanovehicle of claim 1, further comprising at least one targeting ligand attached to the shell, at least one drug, at least one fluorescent dye, at least one targeting moiety, or a combination thereof.

11. (canceled)

12. (canceled)

13. A method of treating, reducing the severity of and/or slowing the progression of a disease, disorder, or disease condition in a subject, comprising:
providing at least one nanovehicle of claim 1;
administering a therapeutically effective amount of the at least one nanovehicle to the subject; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the disease, disorder, or disease condition in the subject.

14. The method of claim 13, wherein the shell further comprises at least one targeting ligand.

15. The method of claim 13, wherein the nanovehicle further comprises at least one drug, or at least one fluorescent dye or both.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A probe comprising at least one coated iron oxide nanoparticle; at least one boron cluster; and at least one targeting moiety.

31. The probe of claim 30, wherein the at least one targeting moiety is attached to the at least one coated iron oxide nanoparticle.

32. The probe of claim 30, wherein the at least one coated iron oxide nanoparticle is selected from the group consisting of Ferumoxytol, Ferumoxides, Ferucarbotran, Ferumoxtran-10, NC100150, VSOP C184, and combinations thereof.

33. The probe of claim 30, further comprising at least one drug, or at least one fluorescent dye, or both.

34. (canceled)

35. (canceled)

36. A method for detecting a cancer in a subject, comprising:
administering an effective amount of at least one probe of claim 30 to the subject, thereby contacting a tissue of the subject with the at least one probe such that the at least one probe binds to the tissue; and
detecting the at least one probe bound to the tissue, wherein the presence of the at least one probe bound to the tissue is indicative of the cancer in the subject.

37. The method of claim 36, further comprising administering a treatment to the subject.

38. The method of claim 13, wherein the disease is a cancer.

39. (canceled)

40. (canceled)

41. (canceled)

42. (canceled)

43. The method of claim 36, wherein the probe is detected by an imaging method selected from the group consisting of magnetic resonance imaging, fluorescence imaging, and combinations thereof.

44. (canceled)

45. The nanovehicle of claim 1, wherein the nanovehicle is a nanoparticle.

46. (canceled)

47. A method of treating, reducing the severity of and/or slowing the progression of cancer in a subject, comprising:
providing at least one nanovehicle, wherein the at least one nanovehicle comprises: a core, wherein the core comprises at least one iron oxide; a shell surrounding the core, wherein the shell comprises at least one polymer; at least one boron cluster; and at least one targeting ligand attached to the shell;
administering a therapeutically effective amount of the at least one nanovehicle to the subject, thereby contacting a tissue of the subject with the at least one nanovehicle,
wherein the tissue is selected from the group consisting of cancerous tissue, non-cancerous tissue, and combinations thereof, and
wherein the nanovehicle selectively binds to the cancerous tissue; and
radiating the at least one nanovehicle with neutrons, wherein the neutrons are selected from low-energy thermal neutrons, thermal neutrons, epithermal neutrons, and combinations thereof, thereby treating, reducing the severity of and/or slowing the progression of the cancer in the subject.

48. The method of claim 47, further comprising at least one drug, or at least one fluorescent dye, or both.

49. (canceled)

50. (canceled)

51. The nanovehicle of claim 10, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.


SEQUENCE LISTING (5 sequences)

SEQ ID NO: 1 - protein, 20 aa, Artificial Sequence
  Synthetic
TFFYGGSRGKRNNFKTEEYC

SEQ ID NO: 2 - protein, 19 aa, Artificial Sequence
  Synthetic
TFFYGGSRGKRNNFKTEEY

SEQ ID NO: 3 - protein, 19 aa, Artificial Sequence
  Synthetic
TFFYGGCRGKRNNFKTEEY

SEQ ID NO: 4 - protein, 19 aa, Artificial Sequence
  Synthetic
TFFYGGSRGKRNNFRTEEY

SEQ ID NO: 5 - protein, 19 aa, Artificial Sequence
  Synthetic
TFFYGGSRGRRNNFRTEEY